SEQUENCE LISTING (108 sequences)

SEQ ID NO: 1 - protein, 39 aa, synthetic construct
AKCEPSQFQCTNGRCITLLWKCDGDEDCVDGSDEKNCVK

SEQ ID NO: 2 - protein, 41 aa, synthetic construct
KTCAESDFVCNNGQCVPSRWKCDGDPDCEDGSDESPEQCHM

SEQ ID NO: 3 - protein, 41 aa, synthetic construct
RTCRIHEISCGAHSTQCIPVSWRCDGENDCDSGEDEENCGN

SEQ ID NO: 4 - protein, 39 aa, synthetic construct
ITCSPDEFTCSSGRCISRNFVCNGQDDCSDGSDELDCAP

SEQ ID NO: 5 - protein, 41 aa, synthetic construct
PTCGAHEFQCSTSSCIPISWVCDDDADCSDQSDESLEQCGR

SEQ ID NO: 6 - protein, 39 aa, synthetic construct
TKCPASEIQCGSGECIHKKWRCDGDPDCKDGSDEVNCPS

SEQ ID NO: 7 - protein, 39 aa, synthetic construct
RTCRPDQFECEDGSCIHGSRQCNGIRDCVDGSDEVNCKN

SEQ ID NO: 8 - protein, 42 aa, synthetic construct
NQCLGPGKFKCRSGECIDISKVCNQEQDCRDWSDEPLKECHI

SEQ ID NO: 9 - protein, 40 aa, synthetic construct
NECNIPGNFMCSNGRCIPGAWQCDGLPDCFDKSDEKECPK

SEQ ID NO: 10 - protein, 60 aa, Eastern equine encephalitis virus
YEHTAVMPNKVGIPYKALVERPGYAPVHLQIQLVNTRIIPSTNLEYITCKYKTKVPSPVV

SEQ ID NO: 11 - protein, 60 aa, Eastern equine encephalitis virus; identical to SEQ ID NO: 10

SEQ ID NO: 12 - protein, 60 aa, Venezuelan equine encephalitis virus
YEHATTMPSQAGISYNTIVNRAGYAPLPISITPTKIKLIPTVNLEYVTCHYKTGMDSPAI

SEQ ID NO: 13 - protein, 60 aa, Semliki Forest virus
YEHSTVMPNVVGFPYKAHIERPGYSPLTLQMQVVETSLEPTLNLEYITCEYKTVVPSPYV

SEQ ID NO: 14 - protein, 60 aa, Chikungunya virus
YEHVTVIPNTVGVPYKTLVNRPGYSPMVLEMELQSVTLEPTLSLDYITCEYKTVIPSPYV

SEQ ID NO: 15 - protein, 60 aa, Eastern equine encephalitis virus
KCCGATQCTSKPHPDYQCQVFSGVYPFMYGGAYCFCDTENTQMSEAYVERSEECSIDHAK

SEQ ID NO: 16 - protein, 60 aa, Eastern equine encephalitis virus
KCCGATQCTSKPHPDYQCQVFTGVYPFMWGGAYCFCDTENTQMSEAYVERSEECSIDHAK

SEQ ID NO: 17 - protein, 60 aa, Venezuelan equine encephalitis virus
KCCGSQECTPTYRPDEQCKVFTGVYPFMWGGAYCFCDTENTQVSKAYVMKSDDCLADHAE

SEQ ID NO: 18 - protein, 59 aa, Semliki Forest virus
KCCGASECSTKEKPDYQCKVYTGVYPFMWGGAYCFCDSENTQLSEAYVDRSDVCHDHAS

SEQ ID NO: 19 - protein, 60 aa, Chikungunya virus
KCCGTAECKDKSLPDYSCKVFTGVYPFMWGGAYCFCDAENTQLSEAHVEKSESCKTEFAS

SEQ ID NO: 20 - protein, 60 aa, Eastern equine encephalitis virus
AYKVHTGTVQAMVNITYGSVSWRSADVYVNGETPAKIGDAKLIIGPLSSAWSPFDNKVVV

SEQ ID NO: 21 - protein, 60 aa, Eastern equine encephalitis virus; identical to SEQ ID NO: 20

SEQ ID NO: 22 - protein, 59 aa, Venezuelan equine encephalitis virus
AYKAHTASVQAFLNITVGEHSIVTTVYVNGETPVNFNGVKITAGPLSTAWTPFDRKIVQ

SEQ ID NO: 23 - protein, 59 aa, Semliki Forest virus
AYKAHTASLKAKVRVMYGNVNQTVDVYVNGDHAVTIGGTQFIFGPLSSAWTPFDNKIVV

SEQ ID NO: 24 - protein, 59 aa, Chikungunya virus
AYRAHTASASAKLRVLYQGNNITVAAYANGDHAVTVKDAKFVVGPMSSAWTPFDNKIVV

SEQ ID NO: 25 - protein, 60 aa, Eastern equine encephalitis virus
YGHEVYNYDFPEYGTGKAGSFGDLQSRTSTSNDLYANTNLKLQRPQAGIVHTPFTQVPSG

SEQ ID NO: 26 - protein, 60 aa, Eastern equine encephalitis virus
YGHEVYNYDFPEYGTGKAGSFGDLQSRTSTSNDLYANTNLKLQRPQAGIVHTPFTQAPSG

SEQ ID NO: 27 - protein, 60 aa, Venezuelan equine encephalitis virus
YAGEIYNYDGPEYGAGQPGAFGDIQSRTVSSSDLYANTNLVLQRPKAGAIHVPYTQAPSG

SEQ ID NO: 28 - protein, 60 aa, Semliki Forest virus
YKDEVFNQDFPPYGSGQPGRFGDIQSRTVESNDLYANTALKLARPSPGMVHVPYTQTPSG

SEQ ID NO: 29 - protein, 60 aa, Chikungunya virus
YKGDVYNMDYPPFGAGRPGQFGDIQSRTPESKDVYANTQLVLQRPAAGTVHVPYSQAPSG

SEQ ID NO: 30 - protein, 60 aa, Eastern equine encephalitis virus
FERWKKDKGAPLNDVAPFGCSIALEPLRAENCAVGSIPISIDIPDAAFTRISETPTVSDL

SEQ ID NO: 31 - protein, 60 aa, Eastern equine encephalitis virus
FERWKRDKGAPLNDVAPFGCSIALEPLRAENCAVGSIPISIDIPDAAFTRISETPTVSDL

SEQ ID NO: 32 - protein, 60 aa, Venezuelan equine encephalitis virus
FEQWKKDKAPSLKFTAPFGCEIYTNPIRAENCAVGSIPLAFDIPDALFTRVSETPTLSAA

SEQ ID NO: 33 - protein, 61 aa, Semliki Forest virus
FKYWLKEKGTALNTAKAPFGCQIKTNPVRAMNCAVGNIPVSMNLPDSAFTRIVEAPTIIDL

SEQ ID NO: 34 - protein, 60 aa, Chikungunya virus
FKYWLKERGASLQHTAPFGCQIATNPVRAVNCAVGNIPISIDIPDAAFTRVVDAPSVTDM

SEQ ID NO: 35 - protein, 55 aa, Eastern equine encephalitis virus
ECKITECTYAFDFGGIATVAYKSSKAGNCPIHSPSGVAVIKENDVTLAESGSFTF

SEQ ID NO: 36 - protein, 55 aa, Eastern equine encephalitis virus
ECKITECTYASDFGGIATVAYKSSKAGNCPIHSPSGVAVIKENDVTLAESGSFTF

SEQ ID NO: 37 - protein, 55 aa, Venezuelan equine encephalitis virus
ECTLNECVYSSDFGGIATVKYSASKSGKCAVHVPSGTATLKEAAVELTEQGSATI

SEQ ID NO: 38 - protein, 55 aa, Semliki Forest virus
TCTVATCTHSSDFGGVLTLTYKTDKNGDCSVHSHSNVATLQEATAKVKTAGKVTL

SEQ ID NO: 39 - protein, 55 aa, Chikungunya virus
SCEVPACTHSSDFGGVAIIKYTASKKGKCAVHSMTNAVTIREADVEVEGNSQLQI

SEQ ID NO: 40 - protein, 58 aa, Eastern equine encephalitis virus
DLDTHFTQKLARPYIADCPNCGHSRCDSPIAIEEVRGDAHAGVIRIQTSAMFGLKTDG

SEQ ID NO: 41 - protein, 59 aa, Eastern equine encephalitis virus
DLDTHFTQYKLARPYIADCPNCGHSRCDSPIAIEEVRGDAHAGVIRIQTSAMFGLKTDG

SEQ ID NO: 42 - protein, 59 aa, Venezuelan equine encephalitis virus
STEELFNEYKLTRPYMARCIRCAVGSCHSPIAIEAVKSDGHDGYVRLQTSSQYGLDSSG

SEQ ID NO: 43 - protein, 60 aa, Semliki Forest virus
SVSQHFNVYKATRPYIAYCADCGAGHSCHSPVAIEAVRSEATKGMLKIQFSAQIGIDKSD

SEQ ID NO: 44 - protein, 56 aa, Chikungunya virus
NFNVYKATRPYLAHCPDCGEGHSCHSPIALERIRNEATDGTLKIQVSLQIGIKTDD

SEQ ID NO: 45 - protein, 58 aa, Eastern equine encephalitis virus
VDLAYMSFMNGKTQKSIKIDNLHVRTSAPCSLVSHHGYYILAQCPPGDTVTVGFHDGP

SEQ ID NO: 46 - protein, 58 aa, Eastern equine encephalitis virus
VKLAYMSFMNGKTQKSIKIDNLHVRTSAPCSLVSHHGYYILAQCPPGDTVTVGFHDGP

SEQ ID NO: 47 - protein, 59 aa, Venezuelan equine encephalitis virus
NLKGRTMRYDMHGTIKEIPLHQVSLYTSRPCHIVDGHGYFLLARCPAGDSITMEFKKDS

SEQ ID NO: 48 - protein, 60 aa, Semliki Forest virus
NHDYTKIRYADGHAIENAVRSSLKVATSGDCFVHGTMGHFILAKCPPGEFLQVSIQDTRN

SEQ ID NO: 49 - protein, 60 aa, Chikungunya virus
SHDWTKLRYMDSHTPADAERAGLLVRTSAPCTITGTMGHFILARCPKGETLTVGFTDSRK

SEQ ID NO: 50 - protein, 60 aa, Eastern equine encephalitis virus
NRHTCTVAHKVEFRPVGREKYRHPPEHGVELPCNRYTHKRADQGHYVEMHQPGLVADHSL

SEQ ID NO: 51 - protein, 60 aa, Eastern equine encephalitis virus; identical to SEQ ID NO: 50

SEQ ID NO: 52 - protein, 60 aa, Venezuelan equine encephalitis virus
VRHSCSVPYEVKFNPVGRELYTHPPEHGVEQACQVYAHDAQNRGAYVEMHLPGSEVDSSL

SEQ ID NO: 53 - protein, 60 aa, Semliki Forest virus
AVRACRIQYHHDPQPVGREKFTIRPHYGKEIPCTTYQQTTAKTVEEIDMHMPPDTPDRTL

SEQ ID NO: 54 - protein, 60 aa, Chikungunya virus
ISHTCTHPFHHEPPVIGRERFHSRPQHGKELPCSTYVQSTAATAEEIEVHMPPDTPDRTL

SEQ ID NO: 55 - protein, 58 aa, Eastern equine encephalitis virus
LSIHSAKVKITVPSGAQVKYYCKCPDVRKGITSSDHTTTCTDVKQCRAYLIDNKKWVY

SEQ ID NO: 56 - protein, 58 aa, Eastern equine encephalitis virus
LSIHSAKVKITVPSGAQVKYYCKCPDVREGITSSDHTTTCTDVKQCRAYLIDNKKWVY

SEQ ID NO: 57 - protein, 60 aa, Venezuelan equine encephalitis virus
VSLSGSSVTVTPPDGTSALVECECGGTKISETINKTKQFSQCTKKEQCRAYRLQNDKWVY

SEQ ID NO: 58 - protein, 57 aa, Semliki Forest virus
LSQQSGNVKITVGGKKVKYNCTCGTGNVGTTNSDMTINTCLIEQCHVSVTDHKKWQF

SEQ ID NO: 59 - protein, 57 aa, Chikungunya virus
MTQQSGNVKITVNGQTVRYKCNCGGSNEGLTTTDKVINNCKIDQCHAAVTNHKNWQY

SEQ ID NO: 60 - protein, 41 aa, Eastern equine encephalitis virus
NSGRLPRGEGDTFKGKLHVPFVPVKAKCIATLAPEPLVEHK

SEQ ID NO: 61 - protein, 41 aa, Eastern equine encephalitis virus; identical to SEQ ID NO: 60

SEQ ID NO: 62 - protein, 41 aa, Venezuelan equine encephalitis virus
NSDKLPKAAGATLKGKLHVPFLLADGKCTVPLAPEPMITFG

SEQ ID NO: 63 - protein, 41 aa, Semliki Forest virus
NSPFVPRADEPARKGKVHIPFPLDNITCRVPMAREPTVIGH

SEQ ID NO: 64 - protein, 42 aa, Chikungunya virus
NSPLVPRNAELGDRKGKIHIPFPLANVTCRVPKARNPTVTYG

SEQ ID NO: 65 - protein, 54 aa, Homo sapiens
GRKAKCEPSQFQCTNGRCITLLWKCDGDEDCVDGSDEKNCVKKTCAESDFVCNN

SEQ ID NO: 66 - protein, 54 aa, Mus musculus
GKKAKCDSSQFQCTNGRCITLLWKCDGDEDCADGSDEKNCVKKTCAESDFVCKN

SEQ ID NO: 67 - protein, 54 aa, Equus caballus
GRKAKCEPSQFQCTNGRCITLLWKCDGDEDCADGSDEKNCVKKTCAESDFVCNN

SEQ ID NO: 68 - protein, 54 aa, Sturnus vulgaris
GARAKCEESQFACGNGRCIPQIWKCDGDEDCSDGSDESACVKKTCAESDFVCLT

SEQ ID NO: 69 - protein, 19 aa, Aedes aegypti
GRASSKETNCSDDKFRCKS

SEQ ID NO: 70 - protein, 19 aa, Aedes albopictus
GRASSKETNCSDDKFRCKS

SEQ ID NO: 71 - protein, 64 aa, Caenorhabditis elegans
APTRKCDATNSFQCQDGRCIPMSWRCDGDIDCQNEEDEKNCPISEVCGAEEHKCGEVKSARSSL

SEQ ID NO: 72 - protein, 59 aa, Homo sapiens
GQCVPSRWKCDGDPDCEDGSDESPEQCHMRTCRIHEISCGAHSTQCIPVSWRCDGENDC

SEQ ID NO: 73 - protein, 59 aa, Mus musculus
GQCVPNRWQCDGDPDCEDGSDESPEQCHMRTCRINEISCGARSTQCIPVSWRCDGENDC

SEQ ID NO: 74 - protein, 59 aa, Equus caballus; identical to SEQ ID NO: 73

SEQ ID NO: 75 - protein, 59 aa, Sturnus vulgaris
GQCVPNRWQCDGDPDCEDGSDESSELCHARTCRVNEISCGPQSTQCIPISWKCDGEKDC

SEQ ID NO: 76 - protein, 59 aa, Aedes aegypti
GRCIPKHWQCDGENDCSDGSDEDSEKCQSKVCSSEEFTCRSGTGTCIPLAWMCDQNRDC

SEQ ID NO: 77 - protein, 59 aa, Aedes albopictus; identical to SEQ ID NO: 76

SEQ ID NO: 78 - protein, 62 aa, Caenorhabditis elegans
ERFKCIPNKWVCDGEFDCEDKSDEFQCKNVSCQEKQFQCEELSGDYSLCIPETWVCDGQRDC

SEQ ID NO: 79 - protein, 54 aa, Homo sapiens
DSGEDEENCGNITCSPDEFTCSSGRCISRNFVCNGQDDCSDGSDELDCAPPTCG

SEQ ID NO: 80 - protein, 54 aa, Mus musculus
DNGEDEENCGNITCSADEFTCSSGRCVSRNFVCNGQDDCDDGSDELDCAPPTCG

SEQ ID NO: 81 - protein, 54 aa, Equus caballus
DSGEDEENCGNITCSPDEFTCSSGRCISRNFVCNGQDDCNDGSDELDCAPPTCG

SEQ ID NO: 82 - protein, 54 aa, Sturnus vulgaris
DSGEDEQNCGNVTCSPADFTCSSGQCISKSFVCNGQDDCSDGSDELECAPPTCG

SEQ ID NO: 83 - protein, 54 aa, Aedes aegypti
PDGSDEMSCNETCRSDEFTCANGRCIQKRWQCDRDDDCGDNSDEKGCQATTCDP

SEQ ID NO: 84 - protein, 54 aa, Aedes albopictus; identical to SEQ ID NO: 83

SEQ ID NO: 85 - protein, 64 aa, Caenorhabditis elegans
TNGKDEQNCTSKTSKCPDNNFQCSNGNCIFKNWVCDGEEDCSDGSDELLTAPSNCNRTVNQCPP

SEQ ID NO: 86 - protein, 61 aa, Homo sapiens
AHEFQCSTSSCIPISWVCDDDADCSDQSDESLEQCGRQPVIHTKCPASEIQCGSGECIHKK

SEQ ID NO: 87 - protein, 61 aa, Mus musculus
AHEFQCSTSSCIPLSWVCDDDADCSDQSDESLEQCGRQPVIHTKCPTSEIQCGSGECIHKK

SEQ ID NO: 88 - protein, 61 aa, Equus caballus
AHEFQCSTSSCIPNSWVCDDDADCSDQSDESLEQCGRQPVIHTKCPASEIQCGSGECIHKK

SEQ ID NO: 89 - protein, 61 aa, Sturnus vulgaris
VHEFQCKSSTCIPLNWVCDDDADCSDHSDESLEQCGRQPAPSVKCSASEVQCGSGECIHKK

SEQ ID NO: 90 - protein, 61 aa, Aedes aegypti
LKQFACSENYCITSKWRCDGEPDCPDGSDERGCTNPTPPTVNPCLSLEYQCSDRITCIHKS

SEQ ID NO: 91 - protein, 61 aa, Aedes albopictus; identical to SEQ ID NO: 90

SEQ ID NO: 92 - protein, 58 aa, Caenorhabditis elegans
GEMWKCGSGECIPSRWRCDAEVDCKDHSDEKNCTAIQHTCKLAEEFACKASHNCINKA

SEQ ID NO: 93 - protein, 49 aa, Homo sapiens
WRCKGDPDCKDGSDEVNCPSRTCRPDQFECEDGSCIHGSRQCNGIRDCV

SEQ ID NO: 94 - protein, 49 aa, Mus musculus
WRCDGDPDCKDGSDEVNCPSRTCRPDQFECEDGSCIHGSRQCNGIRDCV

SEQ ID NO: 95 - protein, 49 aa, Equus caballus; identical to SEQ ID NO: 94

SEQ ID NO: 96 - protein, 49 aa, Sturnus vulgaris
WRCDGDPDCKDGSDEINCPSRTCRPDQFRCEDGNCIHGSRQCNGVRDCL

SEQ ID NO: 97 - protein, 52 aa, Aedes aegypti
WICDGEKDCPQGDDEMPPICQNVTCRPDQFQCKKDKTCINGHFHCNGKPECS

SEQ ID NO: 98 - protein, 52 aa, Aedes albopictus
WICDGEKDCPHGDDEMPPICQNVTCRPDQFQCKKDKTCINGHFHCNGKPECS

SEQ ID NO: 99 - protein, 62 aa, Caenorhabditis elegans
FVCDGELDCSDGSDEDDCADVRTECKSGERTCPASYGAYGAESGHVVCIPASSWCNGEEDCP

SEQ ID NO: 100 - protein, 55 aa, Homo sapiens
DGSDEVNCKNVNQCLGPGKFKCRSGECIDISKVCNQEQDCRDWSDEPLKECHINE

SEQ ID NO: 101 - protein, 55 aa, Mus musculus
DGSDEVNGKNVNQCLGPGKFKCRSGECIDMSKVCDQEQDCRDWSDEPLKECHINE

SEQ ID NO: 102 - protein, 55 aa, Equus caballus
DGSDEVNCKNVNQCLGPGKFKCRSGECIDISKVCNQEQDCRDWSDEPLKECNVNE

SEQ ID NO: 103 - protein, 55 aa, Sturnus vulgaris
DGTDEVNCNNVIQCSGPGKFKCRSGECIDINKVCNQQRDCKDWSDEPLKECNVNE

SEQ ID NO: 104 - protein, 56 aa, Aedes aegypti
DGSDEVDCERPAVKCNPKTEFDCGGGMCIPLSKVCDKKPDCPEFQDEPNDKCGKNE

SEQ ID NO: 105 - protein, 57 aa, Aedes albopictus
DGSDEVDCAERPAVKCNPKTEFDCGGGMCIPLSKVCDKKPDCPEFQDEPNDKCGKNE

SEQ ID NO: 106 - protein, 57 aa, Caenorhabditis elegans
DGGDEKECNMTAPVTCQKGTEYECPSTPLQCIEMSKLCASAQFDCGDGNMSVCSQKK

SEQ ID NO: 107 - DNA, 5934 bp, Synthetic construct
  source 1..5934: mol_type = other DNA
gttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattctccaccatgggatggtcatgtatcatcctttttctagtagcaactgcaaccggtgtacattctgccaaatgtgaaccctcccaattccagtgcacaaatggtcgctgtattacgctgttgtggaaatgtgatggggatgaagactgtgttgacggcagtgatgaaaagaactgtgtaaagaagacgtgtgctgaatctgacttcgtgtgcaacaatggccagtgtgttcccagccgatggaagtgtgatggagatcctgactgcgaagatggttcagatgaaagcccagaacagtgccatatgagaacatgccgcatacatgaaatcagctgtggcgcccattctactcagtgtatcccagtgtcctggagatgtgatggtgaaaatgattgtgacagtggagaagatgaagaaaactgtggcaatggtggcggaagcggtggaagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgat

SEQ ID NO: 108 - DNA, 5811 bp, Synthetic construct
  source 1..5811: mol_type = other DNA
atgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattctccaccatgggatggtcatgtatcatcctttttctagtagcaactgcaaccggtgtacattctgccaaatgtgaaccctcccaattccagtgcacaaatggtcgctgtattacgctgttgtggaaatgtgatggggatgaagactgtgttgacggcagtgatgaaaagaactgtgtaaagaagacgtgtgctgaatctgacttcgtgtgcaacaatggccagtgtgttcccagccgatggaagtgtgatggagatcctgactgcgaagatggttcagatgaaagcccagaacagtgccatatgggtggcggaagcggtggaagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggc

(19) United States
(12) Patent Application Publication
(10) Pub. No.: US 2025/0109199 A1
(43) Pub. Date: Apr. 3, 2025

(54) COMPOSITIONS AND METHODS FOR VLDLR-BASED DECOY RECEPTORS AGAINST ALPHAVIRUSES

(71) Applicants: Michael Diamond, St. Louis, MO (US); Daved Fremont, St. Louis, MO (US); Lucas Adams, St. Louis, MO (US); Saravanan Raju, St. Louis, MO (US)

(72) Inventors: Michael Diamond, St. Louis, MO (US); Daved Fremont, St. Louis, MO (US); Lucas Adams, St. Louis, MO (US); Saravanan Raju, St. Louis, MO (US)

(73) Assignee: Washington University, St. Louis, MO (US)

(21) Appl. No.: 18/905,136

(22) Filed: Oct. 2, 2024

Related U.S. Application Data
(60) Provisional application No. 63/587,367, filed on Oct. 2, 2023.

(51) Int. Cl. C07K 16/28; A61P 31/14; C07K 14/705

(52) U.S. Cl. CPC C07K 16/28; A61P 31/14; C07K 14/705; C07K 2317/52

(57) ABSTRACT

The present disclosure is directed to compositions including a VLDLR-based decoy receptor as, as well as methods of use for preventing and treating alphavirus infection in a subject in need thereof. Compositions of the VLDLR-based decoy receptor include an Fc domain and at least one VLDLR LA domain. A subject in need thereof includes a subject having or at risk for contracting an alphavirus infection selected from at least one of Eastern equine encephalitis virus (EEEV), Chikungunya virus (CHIKV), and Venezuelan equine encephalitis virus (VEEV) infection.

### CROSS-REFERENCE TO RELATED APPLICATIONS

This application claims priority from U.S. Provisional Application Ser. No. 63/587,367 filed on 2 Oct. 2023, which is incorporated herein by reference in its entirety.

MATERIAL INCORPORATED-BY-REFERENCE

The Sequence Listing, which is a part of the present disclosure, includes a computer readable form comprising nucleotide and/or amino acid sequences of the present invention (file name “020745-US-NP_2024-10-02_Sequence-Listing_ST26.xml” created 1 Oct. 2024; 111,295 bytes). The subject matter of the Sequence Listing is incorporated herein by reference in its entirety.

### STATEMENT REGARDING FEDERALLY SPONSORED RESEARCH OR DEVELOPMENT

This invention was made with government support under HHSN272201700060C awarded by the National Institutes of Health, and under MCDC2103-011 awarded by the Defense Threat Reduction Agency (DOD/DTRA). The government has certain rights in the invention.

### FIELD

The present disclosure generally relates to VLDLR-EEEV structures, VLDLR-EEEV binding sites, and VLDLR-based decoy receptors against alphaviruses.

### BACKGROUND

Alphaviruses are arthropod-transmitted, single-stranded positive-sense RNA viruses of the Togaviridae family. These viruses infect a range of vertebrate hosts and have been categorized as “Old World” or “New World” based on geographic origins. Old World alphaviruses include Chikungunya virus (CHIKV), Ross River virus (RRV), Mayaro virus (MAYV), O'nyong-nyong virus (ONNV), and Semliki Forest virus (SFV), many of which cause acute or chronic arthritis. New World alphaviruses, including Eastern equine encephalitis virus (EEEV), Venezuelan equine encephalitis virus (VEEV), and Western equine encephalitis virus (WEEV), cause neurological disease and mortality. EEEV causes sporadic outbreaks across North America, with case fatality rates exceeding 30%.

Though naturally transmitted by mosquitoes, encephalitic alphaviruses also can be spread via aerosolization and have been weaponized in the past. There are no approved countermeasures against infection by encephalitic alphaviruses.

The mature ˜70 nm alphavirus virion is composed of dimerized E1 and E2 glycoproteins, and an internal capsid protein that packages the RNA genome. The virion exhibits T=4 icosahedral symmetry with 240 E1/E2 heterodimers arranged as 80 trimeric spikes on icosahedral three-fold (i3) and quasi-threefold (q3) axes of symmetry. Each asymmetric unit consists of a complete q3 trimer and a single i3 E1/E2 heterodimer. Within each heterodimer, the E2 glycoprotein is preferentially exposed and shields the majority of the E1 glycoprotein from solvent, including the conserved fusion loop required for endosomal escape into the cytoplasm. The B domain of E2 is most distal from the viral membrane, forming the three vertices of the trimeric spike, and the E2 A domain is positioned at the axial interface of the trimer. The E2 A and B domains are principal targets of neutralizing antibodies.

### BRIEF DESCRIPTION OF THE DISCLOSURE

Among the various aspects of the present disclosure are provisions for compositions comprising a VLDLR-based decoy receptor and methods of use thereof.

In one aspect of the present disclosure, a composition including a very-low-density lipoprotein receptor (VLDLR)-based decoy receptor is provided. The VLDLR-based decoy receptor includes: an Fc domain; and at least one VLDLR type A (LA) domain; and wherein the VLDLR-based decoy receptor is an Fc-LA fusion protein.

In some embodiments, the Fc domain is human IgG1. In some embodiments, the at least one VLDLR LA domain is selected from VLDLR-LA1, VLDLR-LA2, VLDLR-LA3, VLDLR-LA5, VLDLR-LA6, and combinations thereof. In some embodiments, the at least one VLDLR LA domain comprises a combination LA domain selected from LA1+LA2, LA1+LA3, LA2+LA5, LA2+LA6, LA3+LA5, and LA5+LA6. In some embodiments, the at least one VLDLR LA domain comprises a tandem LA domain selected from LA(1-2), LA(2-3), LA(3-4), LA(4-5), LA (5-6), and LA(6-7). In some embodiments, the at least one VLDLR LA domain comprises LDLRAD3 LA1. In some embodiments, one or more of the at least one VLDLR LA domains comprise a Trp (W) to Ala (A) mutation. In some embodiments, the VLDLR-based decoy receptor further comprises a VLDLRΔLBD backbone.

In another aspect of the present disclosure, a method for treating alphavirus infection in a subject in need thereof is provided. The method includes: administering to the subject a composition comprising a very-low-density lipoprotein receptor (VLDLR)-based decoy receptor. The VLDLR-based decoy receptor includes: an Fc domain; and at least one VLDLR type A (LA) domain; and wherein the VLDLR-based decoy receptor is an Fc-LA fusion protein.

In some embodiments, the Fc domain is human IgG1 and/or the at least one VLDLR LA domain is selected from: VLDLR-LA1, VLDLR-LA2, VLDLR-LA3, VLDLR-LA5, VLDLR-LA6, and combinations thereof; a combination LA domain selected from LA1+LA2, LA1+LA3, LA2+LA5, LA2+LA6, LA3+LA5, and LA5+LA6; a tandem LA domain selected from LA(1-2), LA(2-3), LA(3-4), LA(4-5), LA (5-6), and LA(6-7); and LDLRAD3 LA1. In some embodiments, one or more of the at least one VLDLR LA domains comprise a Trp (W) to Ala (A) mutation. In some embodiments the VLDLR-based decoy receptor further comprises a VLDLRΔLBD backbone. In some embodiments, the alphavirus is selected from Eastern equine encephalitis virus (EEEV), Chikungunya virus (CHIKV), and Venezuelan equine encephalitis virus (VEEV).

In a further aspect of the present disclosure, a method for preventing an alphavirus infection in a subject is provided. The method includes: administering to the subject a composition comprising a very-low-density lipoprotein receptor (VLDLR)-based decoy receptor. The VLDLR-based decoy receptor includes: an Fc domain; and at least one VLDLR type A (LA) domain; and wherein the decoy receptor is an Fc-LA fusion protein.

In some embodiments, the Fc domain is human IgG1 and/or the at least one VLDLR LA domain is selected from: VLDLR-LA1, VLDLR-LA2, VLDLR-LA3, VLDLR-LA5, VLDLR-LA6, and combinations thereof; a combination LA domain selected from LA1+LA2, LA1+LA3, LA2+LA5, LA2+LA6, LA3+LA5, and LA5+LA6; a tandem LA domain selected from LA(1-2), LA(2-3), LA(3-4), LA(4-5), LA (5-6), and LA(6-7); and LDLRAD3 LA1. In some embodiments, one or more of the at least one VLDLR LA domains comprise a Trp (W) to Ala (A) mutation. In some embodiments the VLDLR-based decoy receptor further comprises a VLDLRΔLBD backbone. In some embodiments, the alphavirus is selected from Eastern equine encephalitis virus (EEEV), Chikungunya virus (CHIKV), and Venezuelan equine encephalitis virus (VEEV).

According to yet another aspect of the present disclosure, a composition comprising a very-low-density lipoprotein receptor (VLDLR)-based decoy receptor is provided. The VLDLR-based decoy receptor comprises an Fc region, a first VLDLR LDLR type A (LA) region, and a second VLDLR LA region, wherein the fusion protein reduces infection by an alphavirus. In one aspect, the VLDLR-based decoy receptor Fc region is a human IgG1. In another aspect, at least one of the first VLDLR LA region and the second VLDLR LA region is selected from VLDLR LA1, VLDLR LA2, VLDLR LA3, VLDLR LA5, and VLDLR LA6. In other aspect the first and second VLDLR LA regions are VLDLR LA1 and VLDLR LA2. In some aspects, the composition comprises a decoy receptor construct of SEQ ID NO: 107. In other aspects, the composition comprises a decoy receptor construct of SEQ ID NO: 108. In another aspect, the alphavirus is selected from an Eastern equine encephalitis virus (EEEV), a chikungunya virus (CHIKV), and a Venezuelan equine encephalitis virus (VEEV).

According to yet an additional aspect of the present disclosure is a method for treating Eastern equine encephalitis virus (EEEV) in a subject in need thereof. The method comprising administering to the subject a composition comprising at least one VLDLR-based decoy receptor. In one aspect, the VLDLR-based decoy receptor comprises a Fc region, a first VLDLR LA region, and a second VLDLR LA region. In another aspect, the VLDLR-based decoy receptor Fc region is a human IgG1. In another aspect, at least one of the first VLDLR LA region and the second VLDLR LA region is selected from VLDLR LA1, VLDLR LA2, VLDLR LA3, VLDLR LA5, and VLDLR LA6. In other aspect the first and second VLDLR LA regions are VLDLR LA1 and VLDLR LA2. In some aspects, the composition comprises a decoy receptor construct of SEQ ID NO: 107. In other aspects, the composition comprises a decoy receptor construct of SEQ ID NO: 108.

According to yet a further aspect of the present disclosure is a method of binding at least one of a surface-displayed alphavirus E1/E2 cleft domain, a E2 A domain, and a E2 B domain. The method comprising administering to a subject with an alphavirus infection, a therapeutically effective amount of a VLDLR-based decoy receptor, wherein the VLDLR-based decoy receptor comprises an (1) Fc region, (2) a first VLDLR LDLR type A (LA) region, and (3) a second VLDLR LA region. In another aspect, the alphavirus is selected from an Eastern equine encephalitis virus (EEEV), a chikungunya virus (CHIKV), and a Venezuelan equine encephalitis virus (VEEV). In another aspect, the VLDLR-based decoy receptor Fc region is a human IgG1. In another aspect, at least one of the first VLDLR LA region and the second VLDLR LA region is selected from VLDLR LA1, VLDLR LA2, VLDLR LA3, VLDLR LA5, and VLDLR LA6. In another aspect, the first VLDLR LA region and the second VLDLR LA region are VLDLR LA1 and VLDLR LA2. In some aspects, the decoy receptor is a construct according to SEQ ID NO: 107. In other aspects, the decoy receptor is a construct according to SEQ ID NO: 108.

Other objects and features will be in part apparent and in part pointed out hereinafter.

### BRIEF DESCRIPTION OF THE DRAWINGS

The application file contains at least one drawing executed in color. Copies of this patent or patent application publication with color drawing(s) will be provided by the Office upon request and payment of the necessary fee.

Those of skill in the art will understand that the drawings, described herein, are for illustrative purposes only. The drawings are not intended to limit the scope of the present teachings in any way.

FIGS. 1(A-C) is an exemplary embodiment of a cryo-EM structure of EEEV PE-6 in complex with VLDLR in accordance with the present disclosure. FIG. 1A is a schematic of Icosahedral reconstructions of EEEV PE-6 VLP alone (upper) or in complex with full-length VLDLR (lower) with 2-fold (i2), 5-fold (i5), 3-fold (i3), and quasi-3-fold (q3) axes designated. Central sections are shown in round insets. Proteins are differentially colored with E1 in tan, E2 A domain in sea green, E2 B domain in blue, the remainder of E2 colored purple, and VLDLR shown in orange. FIG. 1B is a schematic of focused reconstructions of the EEEV asymmetric unit alone (left) or in complex with full-length VLDLR (right). FIG. 1C is an atomic model of a single E1/E2 heterodimer with non-descript LA domains docked into the experimental electron density map, with capsid shown in navy and the lipid bilayer depicted as dashed lines. Interfacial lysines are highlighted in yellow, with the region near K156 magnified in the inset. See also FIG. 9 and FIG. 10(A-D).

FIGS. 2(A-L) is an exemplary embodiment of no single LA domain of the VLDLR LBD being required to support EEEV in accordance with the present disclosure. FIG. 2A is a scheme showing LDLRAD3-LA1 domain replacement of VLDLR LA domains. FIG. 2B is a graph of SINV-EEEV-GFP PE-6 infection of K562 cells transduced with the indicated N-terminal FLAG-tagged constructs and quantified by flow cytometry. FIG. 2C is a schematic of N-terminal VLDLR LA domain truncation constructs. FIG. 2D is a graph SINV-EEEV-GFP PE-6 infection of K562 cells transduced with the indicated N-terminal FLAG-tagged constructs and quantified by flow cytometry. FIG. 2E is a schematic of a single LA domain construct in the context of the VLDLRΔLBD construct. FIG. 2F is a graph of SINV-EEEV-GFP PE-6 infection of K562 cells transduced with the indicated N-terminal FLAG-tagged constructs and quantified by flow cytometry. FIG. 2G is a set of graphs of BLI sensorgrams of biosensors coated with indicated Fc-fusion proteins following incubation with EEEV PE-6 VLPs (left) or biosensors coated with EEEV PE-6 VLPs following incubation with Fc-fusion proteins in solution (right). FIG. 2H is a schematic of VLDLR with Trp (W) to Ala (A) mutations in LA1, LA2, LA3, LA5, and LA6 (left) VLDLR (WA), and also VLDLR (WA) with LA4 (F171W), LA7 (R295W), LA8 (K336W) residues changed to Trp (right). FIG. 2I is a graph of SINV-EEEV-GFP PE-6 infection of K562 cells transduced with the indicated N-terminal FLAG-tagged constructs and quantified by flow cytometry. FIG. 2J is a graph SINV-EEEV-GFP PE-6 infection of K562 cells transduced with variants of VLDLR (WA) in which the indicated single LA domain has been reverted to Trp as indicated. FIG. 2K is a graph SINV-EEEV-GFP PE-6 infection of K562 cells transduced with variants of VLDLR (WA) in which the indicated two LA domains have been reverted to Trp as indicated. FIG. 2L is a schematic a tandem LA domain construct and an accompanying graph of SINV-EEEV-GFP PE-6 infection of K562 cells transduced with the indicated tandem LA domain constructs in the context of the VLDLRΔLBD backbone. Data in FIG. 2B, FIG. 2D, FIG. 2F, and FIGS. 2(I-L) are pooled from two to six experiments. Data in (FIG. 2G) are representative of two experiments. *p<0.05, ****p<0.0001, n.s., not significant; one-way ANOVA with Dunnett's posttest. See also FIGS. 11(A-H), FIGS. 12(A-I), and FIGS. 16(A-B).

FIGS. 3(A-J) is an exemplary embodiment of multiple LA domains mediating the neutralization of EEEV by VLDLR decoys in accordance with the present disclosure. FIG. 3A is a graph of the infection of 293T cells by SINV-EEEV-GFP PE-6 following pre-incubation with the indicated Fc-fusion proteins (10 mg/mL) prior to inoculation. GFP expression was measured by flow cytometry. FIG. 3B is a graph of a dose response curve of neutralization by Fc-fusion proteins against SINV-EEEV-GFP PE-6. FIG. 3C is a graph of the infection of 293T cells by SINV-EEEV-GFP PE-6 following pre-incubation with the indicated Fc-fusion proteins (10 mg/mL) prior to inoculation. GFP expression was measured by flow cytometry. FIG. 3D is a graph of a dose response curve of neutralization by Fc-fusion proteins against SINV-EEEV-GFP PE-6. FIG. 3E is a graph of steady-state BLI curve of LA(1-2) VLDLR domain bound to EEEV PE-6 VLP coated biosensors. FIG. 3F is a graph of steady-state BLI curve of LA(2-3) VLDLR domain bound to EEEV PE-6 VLP coated biosensors. FIG. 3G is a graph of steady-state BLI curve of LA(1-3) VLDLR domain bound to EEEV PE-6 VLP coated biosensors. FIG. 3H is a graph of steady-state BLI curve of LA(1-4) VLDLR domain bound to EEEV PE-6 VLP coated biosensors. FIG. 3I is a graph of steady-state BLI curve of LA(1-5) VLDLR domain bound to EEEV PE-6 VLP coated biosensors. FIG. 3J is a graph of steady-state BLI curve of LA(1-6) VLDLR domain bound to EEEV PE-6 VLP coated biosensors. Data in FIG. 3A and FIG. 3C are pooled from three to six experiments. **p<0.01, ****p<0.0001, n.s., not significant by one-way ANOVA with Dunnett's post test. Data in FIG. 3B and FIG. 3D are representative of three experiments with mean half-maximal effective inhibitory concentrations (EC50 values) calculated. Data in FIGS. 3(E-J) are pooled from three experiments. See also FIGS. 16(A-B).

FIGS. 4(A-H) is an exemplary embodiment of cryo-EM structure of EEEV PE-6 VLPs in complex with VLDLR LA(1-2) in accordance with the present disclosure. FIG. 4A is a graphical representation of the EEEV PE-6 asymmetric unit in complex with VLDLR LA(1-2). E1, tan; E2 A domain, green; E2 B domain, blue; remainder of E2, purple; and VLDLR, orange. FIG. 4B is a graphical representation of the individual E1/E2 heterodimers at the binding interface, illustrating conventional wrapped and intraspike contacts. E1, tan; E2 A domain, green; E2 B domain, blue; remainder of E2, purple; and VLDLR, orange. FIG. 4C is a graphical representation of VLDLR LA(1-2) (orange) overlaying a surface representation of neighboring E1/E2 heterodimers. E1, tan; E2 A domain, green; E2 B domain, blue; remainder of E2, purple; and VLDLR, orange. FIG. 4D is a magnified graphical representation of FIG. 4C (box i) showing the interface details between VLDLR LA1 and the E1 fusion loop (pale green). VLDLR residues, in white or orange; EEEV residues, in black. Predicted salt bridges (interatomic distance % 4.0 Å) and cation-p interactions (% 6.0 Å from aromatic plane) are demarcated by white or yellow dashed lines, respectively. FIG. 4E is a magnified graphical representation of FIG. 4C (box ii) showing the interface details between VLDLR LA1 and E2. VLDLR residues, in white or orange; EEEV residues, in black. Predicted salt bridges (interatomic distance % 4.0 Å) and cation-p interactions (% 6.0 Å from aromatic plane) are demarcated by white or yellow dashed lines, respectively. FIG. 4F is a magnified graphical representation of FIG. 4C (box iii) showing the interface details between VLDLR LA2 and E2. VLDLR residues, in white or orange; EEEV residues, in black. Predicted salt bridges (interatomic distance % 4.0 Å) and cation-p interactions (% 6.0 Å from aromatic plane) are demarcated by white or yellow dashed lines, respectively. FIG. 4G is a graph of the binding of LA(1-2)-Fc to captured wild-type (WT) and mutant EEEV FL93-939 VLPs. Biosensors were coated with WT or mutant VLPs followed by incubation with 1 mM of LA(1-2)-Fc for 300 s. Binding was calculated as percent signal (Rₘₐₓ) relative to WT VLPs. FIG. 4H is a graph of the infection of K562 cells expressing WT and mutant constructs of VLDLR LA(1-2) by SINV-EEEV PE-6 as measured by flow cytometry. Data in FIG. 4G and FIG. 4H are pooled from two to four experiments. ****p<0.0001, n.s., not significant by one-way ANOVA with Dunnett's post test. See also FIGS. 16(A-B), Table 1, and Table 2.

FIGS. 5(A-F) is an exemplary embodiment of mapping the LA domain and EEEV E2 binding sites in accordance with the present disclosure. FIG. 5A is a schematic of cryo-EM reconstruction of EEEV PE-6 or FL93-939 VLP in complex with different VLDLR fragments. VLDLR constructs include full-length VLDLR; LA(1-2); LA(1-3); LA(1-5mut3); LA(1-6mut3,5); LBDmut3,5,6; LA(3-8); LA(1-6mut2); and LA(1-6). EEEV E1, tan; E2 A domain, dark green; E2 B domain, blue; remainder of E2, purple; and VLDLR, orange.

FIG. 5B is a graphical representation depicting the interaction of VLDLR LA6 with EEEV E1 and E2. EEEV E1, tan; E2 A domain, dark green; E2 B domain, blue; remainder of E2, purple; VLDLR, orange; and fusion loop (FL) shown in pale green. Predicted salt bridges (interatomic distance % 4.0 Å) are demarcated by white dashed lines. FIG. 5C is a graph of the infection of SINV-EEEV-GFP in PE-6 WT and mutant K562 cells expressing WT VLDLR as quantified by flow cytometry. FIG. 5D is a graph of the infection of SIVN-EEEV-GFP in PE-6 WT and mutant K562 cells expressing WT VLDLR as quantified by flow cytometry. FIG. 5E is a graph of dose-response neutralization (10, 1, and 0.1 mg/mL) of 30 indicated Fc-fusion proteins against SINV-EEEV PE-6 HKR→AAA virus in 293T cells. FIG. 5F is a graph of the infection of SIVN-EEEV-GFP in PE-6 WT and mutant K562 cells expressing WT VLDLR as quantified by flow cytometry. Data in FIG. 5C, FIG. 5D, and FIG. 5F are pooled from three to six experiments. Data in FIG. 5E are representative of two experiments. ***p<0.001, ****p<0.0001; one-way ANOVA with Dunnett's post test. See also FIGS. 13(A-I), FIGS. 16(A-B), FIG. 17, and FIGS. 18(A-B), Table 1, and Table 2.

FIGS. 6(A-F) is an exemplary embodiment of a comparative analysis of alphavirus-receptor complexes in accordance with the present disclosure. FIG. 6A is a graphical representation of the EEEV:VLDLR alphavirus-receptor complex, with VLDLR (orange; strain-specific site shown as transparent) displayed as a ribbon diagram, overlaying surface representation of EEEV. E1, tan; E2 A domain, green; E2 B domain, blue; and remainder of E2, pale purple. FIG. 6B is a graphical representation of the VEEV:LDLRAD3 LA1 alphavirus-receptor complex, with LDLRAD3 LA1 (yellow; strain-specific site shown as transparent) displayed as a ribbon diagram, overlaying surface representation of VEEV. E1, tan; E2 A domain, green; E2 B domain, blue; and remainder of E2, pale purple. FIG. 6C is a graphical representation of the CHIKV:MXRA8 alphavirus-receptor complex, with LDLRAD3 LA1 (magenta; strain-specific site shown as transparent) displayed as a ribbon diagram, overlaying surface representation of CHIKV. E1, tan; E2 A domain, green; E2 B domain, blue; and remainder of E2, pale purple. FIG. 6D is a graphical representation of the SFV:VLDLR LA3 alphavirus-receptor complex, with LDLRAD3 LA1 (green; strain-specific site shown as transparent) displayed as a ribbon diagram, overlaying surface representation of SFV. E1, tan; E2 A domain, green; E2 B domain, blue; and remainder of E2, pale purple. FIG. 6E is a magnified graphical representation showing the overlay of VLDLR LA1 (orange) and LDLRAD3 LA1 (yellow) within the EEEV/VEEV receptor-binding cleft. FIG. 6F show surface representations of neighboring E1/E2 heterodimers with receptor-binding interfaces highlighted on the respective alphaviruses (EEEV, SFV, VEEV, CHIKV). E1, tan; E2 A domain, green; E2 B domain, blue; and remainder of E2, pale purple. See also FIGS. 14(A-B), FIGS. 15(A-B), FIG. 17, and FIGS. 18(A-B).

FIGS. 7(A-D) is an exemplary embodiment of VLDLR LA(1-2)-Fc protecting against EEEV FL93-939 challenge in accordance with the present disclosure. FIG. 7A is a graph of the survival of CD-1 mice after administration of 100 μg of indicated Fc-fusion protein (PBS, LDLRAD3-LA1-Fc, VLDLR LA(1-2)-Fc, VLDLR LBD-Fc) prior to subcutaneous challenge with EEEV FL93-939. FIG. 7A log rank test with Bonferroni correction. FIG. 7B is a graph of the weight change in CD-1 mice after administration of 100 μg of indicated Fc-fusion protein (PBS, LDLRAD3-LA1-Fc, VLDLR LA(1-2)-Fc, VLDLR LBD-Fc) prior to subcutaneous challenge with EEEV FL93-939. FIG. 7C is a graph of the survival of CD-1 mice after administration of 100 μg of indicated Fc-fusion protein (PBS, LDLRAD3-LA1-Fc, VLDLR LA(1-2)-Fc, VLDLR LBD-Fc) prior to aerosol challenge with EEEV FL93-939.

FIG. 7C log rank test: **p<0.01, ****p<0.0001. FIG. 7D is a set of graph of clinical scores of CD-1 mice administered 100 μg of the fusion protein LDLRAD3-LA1-Fc (top), VLDLR-LBD-Fc (middle), or VLDLR LA(1-2)-Fc (bottom) prior to subcutaneous challenge with EEEV FL93-939. Healthy, white; Ruffled Fur, green; Hunched, blue; Seizures/Ataxia, yellow; Moribund, red; Dead, black. Two experiments with n=10 mice per group. The scoring system is described herein elsewhere. See also FIGS. 13(A-I).

FIG. 8 is a graphical representation of the structural and function basis of VLDLR usage by EEEV. EEEV enters a hots cell by binding to multiple LA domains of VLDLR (top). Administration of a protective soluble decoy receptor protects against lethal EEEV challenge in mice models (bottom).

FIG. 9 is an exemplary embodiment of cryo-EM methodology in accordance with the present disclosure. Images and graphical representations depict the Cryo-EM data processing steps for EEEV VLP (left), EEEV VLP+full-length VLDLR (middle), and EEEV VLP+VLDLR LA(1-2) (right). Related to FIGS. 1(A-C).

FIG. 10(A-D) is an exemplary embodiment of cryo-EM quality control in accordance with the present disclosure. FIG. 10A is a graphical representation of local resolution estimates (top) and graph (bottom) of FSC of EEEV asymmetric unit apo. Resolutions were estimated in cryoSPARC using a 0.143 FSC cutoff. FIG. 10B is a graphical representation of local resolution estimates (top) and graph (bottom) of FSC of EEEV asymmetric unit apo in complex with full-length VLDLR. Resolutions were estimated in cryoSPARC using a 0.143 FSC cutoff. FIG. 10C is a graphical representation of local resolution estimates (top) and graph (bottom) of FSC of EEEV asymmetric unit apo in complex with VLDLR LA(1-2). Resolutions were estimated in cryoSPARC using a 0.143 FSC cutoff. FIG. 10D is a graphical representation of example model fit at EEEV-VLDLR interfaces with experimental cryo-EM densities shown as a mesh. Related to FIGS. 1(A-C).

FIGS. 11(A-H) is an exemplary embodiment of the defining VLDLR LA domains that support EEEV PE-6 binding and infection FIG. 11A is a phylogenetic tree of the alphaviruses MXRA8 (blue), LDLRAD3 (yellow), and VLDLR/ApoER2 (green) generated using the sequences of the structural genes E1 and E2 of the indicated alphaviruses. Colored lines indicate known receptor usage by the corresponding virus. See also FIGS. 2(A-L). FIG. 11B is a set graphs of flow cytometry plots of K562 cells stained with mAbs (Isotype, α-VLDLR) (left) and representative flow plots of GFP expression in K562 cells following SINV-EEEV-GFP expression (right). FIG. 11C is a set of flow cytometry histograms showing expression of the indicated FLAG-tagged VLDLR constructs in K562 cells. FIG. 11D is a schematic of SINV chimeric reporter viruses in which the structural genes of SINV have been replaced with those of the indicated alphaviruses in addition to GFP. FIG. 11E is a schematic (left) and corresponding graph (right) of infected K562 cells expressing the indicated constructs by SINV-VEEV-GFP as assessed by GFP expressing using flow cytometry. Data are pooled from two to four independent experiments. FIG. 11G is a set of flow cytometry histograms showing expression of the indicated FLAG-tagged truncated VLDLR constructs in K562 cells. FIG. 11G is a set of flow cytometry histograms of the indicated single LA domain transduced K562 cells showing expression of the FLAG tag in control K562 and K562-VLDLR cells. FIG. 11H is an image of the alignment of 8 LA domains of VLDLR and LDLRAD3 LA1, numbered according to VLDLR LA1 and corresponding to SEQ ID NO: 1-9. Filled or open arrowheads respectively indicate residues that coordinate calcium by side-chain or main-chain carbonyl. Related to FIGS. 2(A-L).

FIGS. 12 (A-I) is an exemplary embodiment of characterization of LA domain binding of VLDLR by EEEV PE-6 in accordance with the present disclosure. FIG. 12A is a schematic of biolayer interferometry (BLI) experiments in which Fc-fusion proteins are captured with anti-human Fc biosensors followed by incubation with VLPs (left, “receptor immobilized”), and VLPs are captured by anti-mouse mAbs (EEEV-3) follow by incubation with Fc-fusion proteins (right, “receptor in solution”). FIG. 12B is a set of graphs of BLI of Fc-fusion proteins following incubation with VLPs. Representative sensor traces are shown after dipping into wells containing 20 mg/mL of EEEV (left) or VEEV VLP (right). Data are representative of two independent experiments. FIG. 12C is a set of flow cytometry histograms showing expression of the indicated FLAG-tagged chimeric VLDLR constructs in K562 cells. FIG. 12D is a schematic (left) and corresponding graph (right) quantifying percent GFP+ cells 24 h after SINV-VEEV-GFP TrD infection of K562 cells expressing indicated VLDLR(WA) constructs. FIG. 12E is a set of flow cytometry histograms showing expression of the indicated FLAG-tagged chimeric VLDLR constructs in K562 cells. FIG. 12F is a set of flow cytometry histograms showing expression of the indicated FLAG-tagged chimeric VLDLR constructs in K562 cells. FIG. 12G is a set of flow cytometry histograms showing expression of the indicated FLAG-tagged chimeric VLDLR constructs in K562 cells. FIG. 12H is a graph quantifying the percent of GFP+ cells 4 h after SINV-SFV4-GFP infection of K562 cells expressing WTVLDLR or LA(2-3)ΔLBD. Data are pooled from 4 independent experiments. FIG. 12I is a graph of FLAG expression of indicated VLA(1-2)DLBD mutants as assessed by flow cytometry. Related to FIGS. 2(A-L).

FIGS. 13(A-I) is an exemplary embodiment of characterization of EEEV FL93-939 binding and infection by VLDLR in accordance with the present disclosure. FIG. 13A is a set of graphs showing dose-response curves of neutralization by the indicated Fc-fusion proteins against SINV-EEEV FL93-939-GFP. FIG. 13B is a set of graphs showing steady-state BLI curves of monovalent LA(1-6) bound to indicated EEEV FL93-939 VLP-coated biosensors. Data are pooled from three independent experiments. FIG. 13C is a graph of BLI of Fc fusions following incubation with VLPs. Representative sensor traces are shown after dipping into wells containing 20 mg/mL of EEEV FL93-939 VLPs. FIG. 13D is a graph of SINV-EEEV-GFP FL93-939 infection of K562 cells transduced with variants of VLDLR (WA) in which a single LA domain has been reverted to Trp. FIG. 13E is a graph of SINV-EEEV-GFP FL93-939 infection of K562 cells transduced with variants of VLDLR (WA) in which two LA domains have been reverted to Trp. FIG. 13F is a schematic showing the LA(1-6)-Fc fusion proteins and the relevant Trp residues are annotated. FIG. 13G is a graph showing the neutralization of the indicated Trp variants of LA(1-6)-Fc fusion proteins against SINV-EEEV-GFP PE-6 in 293T cells. FIG. 13H is a graph showing the neutralization of the indicated Trp variants of LA(1-6)-Fc fusion proteins against SINV-EEEV-GFP FL93-939 in 293T cells. FIG. 13I is a graph of LA(1-6mut2)-Fc binding to captured wild-type (WT) and mutant EEEV FL93-939 VLPs. Biosensors were coated with WT or mutant VLPs followed by incubation with 25 nM of LA(1-6mut2)-Fc for 300 s. Binding was calculated as percent signal (Rₘₐₓ) relative to WT VLPs. Related to FIGS. 5(A-F) and FIGS. 7(A-D).

FIGS. 14(A-B) is an exemplary embodiment of alphavirus E1 multiple sequence alignments with receptor contacts, corresponding to SEQ ID NO: 10-39, in accordance with the present disclosure. FIG. 14A shows structural alignments of E1 proteins (β1-β14) from EEEV PE-6 GenBank: L37662.1), EEEV FL93-939 (GenBank: EF151502.1), VEEV (strain TC-83, GenBank: AAB02517.1), SFV (strain 4, GenBank: AKC01668.1), and CHIKV (strain 37997, GenBank: AAU43881.1) generated with PROMALS3D and visualized with ESPript 3. Receptor contacts (determined by PISA) are shown below the alignment in orange (EEEV/VLDLR; transparent orange for PE-6-specific contacts), yellow (VEEV/LDLRAD3), purple (CHIKV/MXRA8), or green (SFV/VLDLR), delineated as wrapped, intraspike, interspike (CHIKV/MXRA8 only), or vertex (SFV/VLDLR only). FIG. 14B shows structural alignments of E1 proteins (P15-P28) from EEEV PE-6 GenBank: L37662.1), EEEV FL93-939 (GenBank: EF151502.1), VEEV (strain TC-83, GenBank: AAB02517.1), SFV (strain 4, GenBank: AKC01668.1), and CHIKV (strain 37997, GenBank: AAU43881.1) generated with PROMALS3D and visualized with ESPript 3. Receptor contacts (determined by PISA) are shown below the alignment in orange (EEEV/VLDLR; transparent orange for PE-6-specific contacts), yellow (VEEV/LDLRAD3), purple (CHIKV/MXRA8), or green (SFV/VLDLR), delineated as wrapped, intraspike, interspike (CHIKV/MXRA8 only), or vertex (SFV/VLDLR only). Blue boxes highlight electropositive residues on a given virus known to form salt bridges with the calcium-coordination site of an LA domain receptor. Related to FIGS. 6(A-F).

FIGS. 15(A-B) is an exemplary embodiment of alphavirus E2 multiple sequence alignments with receptor contacts, corresponding to SEQ ID NO: 40-64, in accordance with the present disclosure. FIG. 15A shows structural alignments of E2 proteins from EEEV PE-6 (GenBank: L37662.1), EEEV FL93-939 (GenBank: EF151502.1), VEEV (strain TC-83, GenBank: AAB02517.1), SFV (strain 4, GenBank: AKC01668.1), and CHIKV (strain 37997, GenBank: AAU43881.1) generated with PROMALS3D and visualized with ESPript 3. Receptor contacts (determined by PISA) are shown below the alignment in orange (EEEV/VLDLR; transparent orange for PE-6-specific contacts), yellow (VEEV/LDLRAD3), or purple (CHIKV/MXRA8), delineated as wrapped, intraspike, or interspike (CHIKV/MXRA8 only). Blue boxes highlight electropositive residues on a given virus known to form salt bridges with the calcium-coordination site of an LA domain receptor. FIG. 15B shows structural alignments of E2 proteins from EEEV PE-6 (GenBank: L37662.1), EEEV FL93-939 (GenBank: EF151502.1), VEEV (strain TC-83, GenBank: AAB02517.1), SFV (strain 4, GenBank: AKC01668.1), and CHIKV (strain 37997, GenBank: AAU43881.1) generated with PROMALS3D and visualized with ESPript 3. Receptor contacts (determined by PISA) are shown below the alignment in orange (EEEV/VLDLR; transparent orange for PE-6-specific contacts), yellow (VEEV/LDLRAD3), or purple (CHIKV/MXRA8), delineated as wrapped, intraspike, or interspike (CHIKV/MXRA8 only). Blue boxes highlight electropositive residues on a given virus known to form salt bridges with the calcium-coordination site of an LA domain receptor. Related to FIGS. 6(A-F).

FIGS. 16(A-B) is an exemplary embodiment of differential VLDLR LA domain usage by distinct binding sites on EEEV in accordance with the present disclosure. FIG. 16A is a schematic representation of VLDLR LA domain usage at the different receptor-binding sites on EEEV (E1/E2 cleft, E2 A domain, and E2 B domain). The cleft and E2 A sites are conserved in all EEEV strains, whereas the E2 B domain binding site is present in the few strains featuring residue E2-206K (e.g., EEEV PE-6). Arrows from LA domains indicate which sites on EEEV are bound, respectively, with solid lines indicating interactions observed structurally, and thicker lines indicating interactions that are of higher affinity. FIG. 16B is a schematic representation of predictive EEEV-VLDLR-binding modes on the virion surface. Related to FIGS. 2(A-L), FIGS. 3(A-J), FIGS. 4(A-H), and FIGS. 5(A-F).

FIG. 17 is an exemplary embodiment of engagement of LA domains by different viruses in accordance with the present disclosure. Shown are ribbon diagrams and surface renderings of the LA-binding interfaces for EEEV, SFV (PDB: 81HP), VEEV (PDB: 7FFF), human rhinovirus 2 (HRV2, PDB: 3DPR), and vesicular stomatitis virus (VSV, PDB: 50YL). Related to FIGS. 5(A-F) and FIGS. 6(A-F).

FIGS. 18(A-B) is an exemplary embodiment of sequence alignments of VLDLR orthologs, corresponding to SEQ ID NO: 65-106, in accordance with the present disclosure. FIG. 18A shows sequence alignments of Homo sapiens (human, GenBank: NP_003374.3), Mus musculus (mouse, GenBank: NP_038731.2), Equus caballus (horse, GenBank: XP_023483037.1), Sturnus vulgaris (avian, GenBank: XP_014736085.1), Aedes aegypti (mosquito, GenBank: AEY84776.1), Aedes albopictus (mosquito, GenBank: JAC13440.1), and Caenorhabditis elegans (nematode, GenBank: NP_872023.2) VLDLR orthologs. Structural homology-guided alignment was performed via cysteine barcoding followed by alignment with PROMALS3D, visualized using ESPript 3. LA domains are annotated below the alignment. Predicted EEEV contacts are designated by large (close contacts) or small (other contacts) dots, with strain-specific contacts (in LA6) colored lightly. FIG. 18B shows sequence alignments of Homo sapiens (human, GenBank: NP_003374.3), Mus musculus (mouse, GenBank: NP_038731.2), Equus caballus (horse, GenBank: XP_023483037.1), Sturnus vulgaris (avian, GenBank: XP_014736085.1), Aedes aegypti (mosquito, GenBank: AEY84776.1), Aedes albopictus (mosquito, GenBank: JAC13440.1), and Caenorhabditis elegans (nematode, GenBank: NP_872023.2) VLDLR orthologs. Structural homology-guided alignment was performed via cysteine barcoding followed by alignment with PROMALS3D, visualized using ESPript 3. LA domains are annotated below the alignment. Predicted EEEV contacts are designated by large (close contacts) or small (other contacts) dots, with strain-specific contacts (in LA6) colored lightly. Related to FIGS. 5(A-F) and FIGS. 6(A-F).

### DETAILED DESCRIPTION OF THE DISCLOSURE

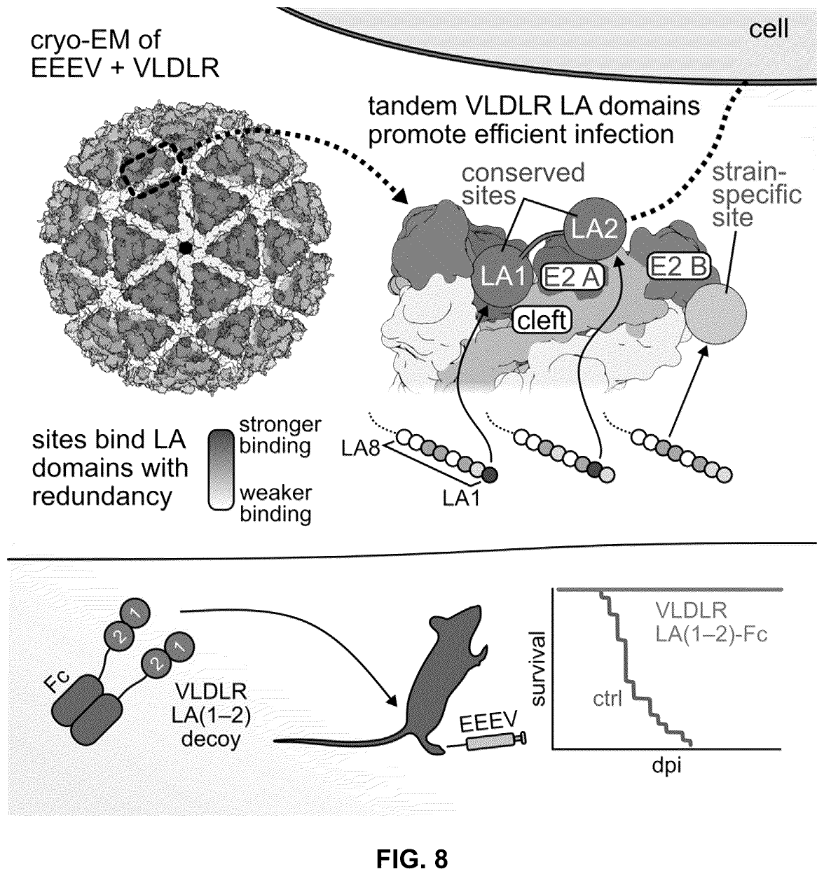

Disclosed herein is the cryo-EM structure of the alphavirus receptor VLDLR bound to virions of the Eastern equine encephalitis virus (EEEV). This structural information was used to design soluble receptor decoy proteins (e.g., VLDLR LA1-LA2-Fc) that neutralize EEEV and protect against infection and disease in vivo in challenge models in mice (FIG. 8).

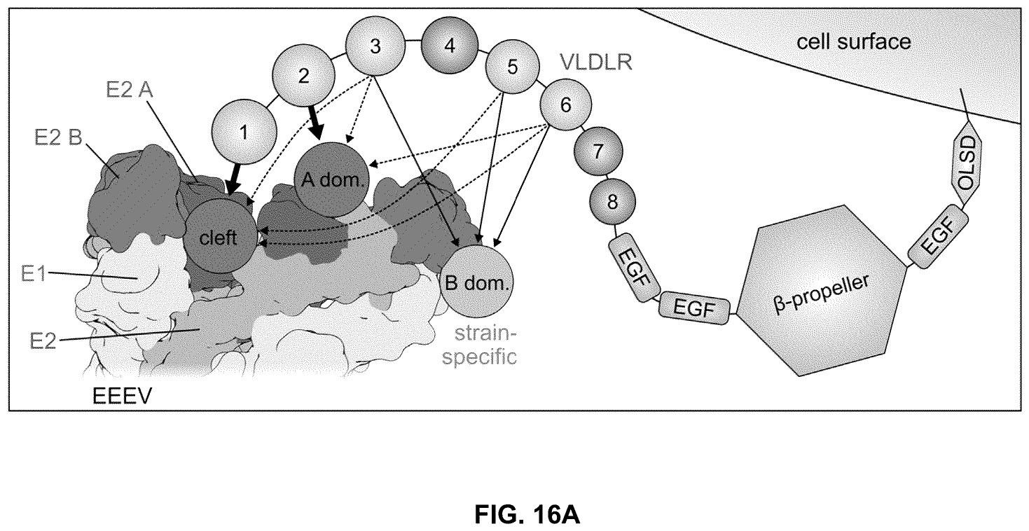

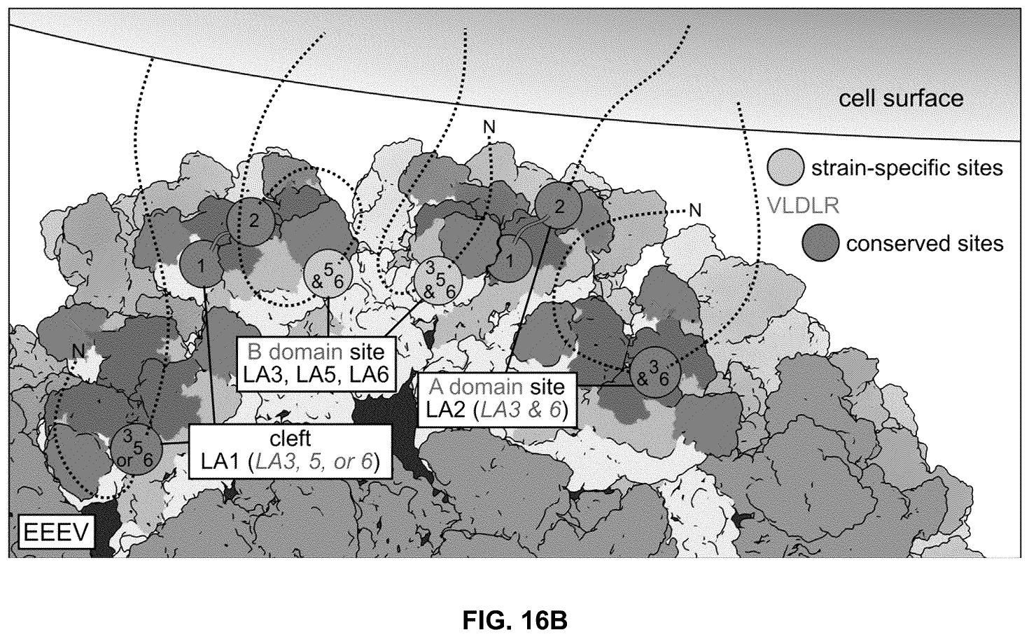

Eastern equine encephalitis virus (EEEV) enters host cells by binding to multiple LA domains of very-low-density lipoprotein receptor (VLDLR) at different sites on the viral glycoproteins. This mode of engagement is distinct from previously described alphavirus-receptor interactions and informs the generation of a protective soluble decoy receptor. Disclosed herein is a model that shows that EEEV uses multiple distinct sites on the E2 glycoprotein to mediate efficient VLDLR-dependent infection (FIG. 16A, FIG. 16B). The five VLDLR LA domains (LA1, LA2, LA3, LA5, and LA6) that encode a conserved Trp all can participate in binding to EEEV, and there is promiscuity in LA domain interactions with each of the three EEEV binding sites.

EEEV engages VLDLR LA1 and LA2 concurrently, with LA1 bound within the cleft near the E1 FL and LA2 positioned atop a neighboring site on the A domain of E2. LA3, LA5, and LA6 all can bind to the E2 B domain site when the K206 residue is present in EEEV. Domain(s) other than LA1 can bind in the cleft, and infection or neutralization of FL93-939 (which lacks the B domain site) with an LA(1-6) featuring an inactivating LA2 W89 mutation indicates an additional LA domains can bind the E2-A domain site. EEEV engages VLDLR LA(1-2) similar to CHIKV and VEEV, as it dominantly interacts with sites on E2 and the E1 FL within the E1/E2 cleft. This differential engagement of LA domains through different envelope protein sites indicates how distantly related alphaviruses can bind the same host receptor and LDL-receptor family members also are implicated in entry of viruses from unrelated families.

### Inhibiting Agent

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| | | Bases | Complementary |
|---|---|---|---|
| Base | Name | Represented | Base |
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2 Å) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged | D E |
| (Acidic): | |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing EEEV (or similar alphavirus infection) in a subject in need thereof by administration of a therapeutically effective amount of a VLDLR-based decoy receptor.

Methods described herein are generally performed on a subject in need thereof. A subject in need of the therapeutic methods described herein can be a subject having, diagnosed with, suspected of having, or at risk for developing EEEV (or similar alphavirus infection). A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a VLDLR-based decoy receptor is, for example, an amount that would cause the desired therapeutic effect in a subject while minimizing undesired side effects. In various embodiments, an effective amount of a VLDLR-based decoy receptor described herein can substantially inhibit, slow the progress of, or limit the development of EEEV (or similar alphavirus infection).

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a VLDLR-based decoy receptor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat EEEV (or other alphavirus infection).

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a VLDLR-based decoy receptor can occur as a single event or over a time course of treatment. For example, a VLDLR-based decoy receptor can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for EEEV (or other alphavirus infection).

A VLDLR-based decoy receptor can be administered simultaneously or sequentially with another agent, such as an antibiotic, an anti-inflammatory, or another agent. For example, a VLDLR-based decoy receptor can be administered simultaneously with another agent, such as an antibiotic or an anti-inflammatory. Simultaneous administration can occur through administration of separate compositions, each containing one or more of a VLDLR-based decoy receptor, an antibiotic, an anti-inflammatory, or another agent. Simultaneous administration can occur through administration of one composition containing two or more of a VLDLR-based decoy receptor, an antibiotic, an anti-inflammatory, or another agent. A VLDLR-based decoy receptor can be administered sequentially with an antibiotic, an anti-inflammatory, or another agent. For example, a VLDLR-based decoy receptor can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, a VLDLR-based decoy receptor may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a VLDLR-based decoy receptor may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cryotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to precursors, reagents, and/or equipment/apparatus. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Structural and Functional Basis of VLDLR Usage by Eastern Equine Encephalitis Virus

### Summary

The very low-density lipoprotein receptor (VLDLR) is comprised of eight low-density lipoprotein receptor (LDLR) type A (LA) domains and supports entry of the distantly related Eastern equine encephalitis (EEEV) and Semliki Forest (SFV) alphaviruses. Here, by resolving multiple cryo-electron microscopy structures of EEEV-VLDLR complexes and performing mutagenesis and functional studies, it is herein shown that EEEV uses multiple sites (E1/E2 cleft and E2 A domain) to engage more than one LA domain simultaneously. However, no single LA domain is necessary or sufficient to support efficient EEEV infection. Whereas all EEEV strains show conservation of two VLDLR binding sites, the EEEV PE-6 strain and a few other EEE complex members feature a single amino acid substitution that enables binding of LA domains to an additional site on the E2 B domain. These structural and functional analyses informed the design of a minimal VLDLR decoy receptor that neutralizes EEEV infection and protects mice from lethal challenge.

### Results

Structure of EEEV in Complex with Full-Length VLDLR.

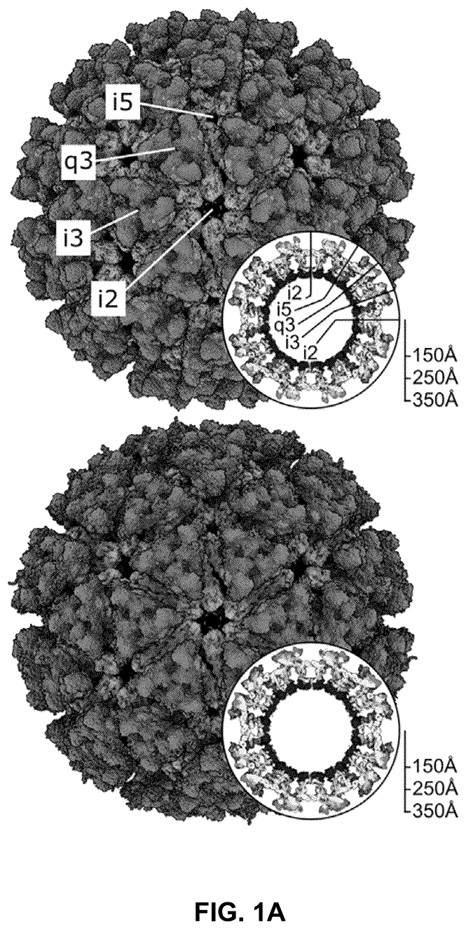

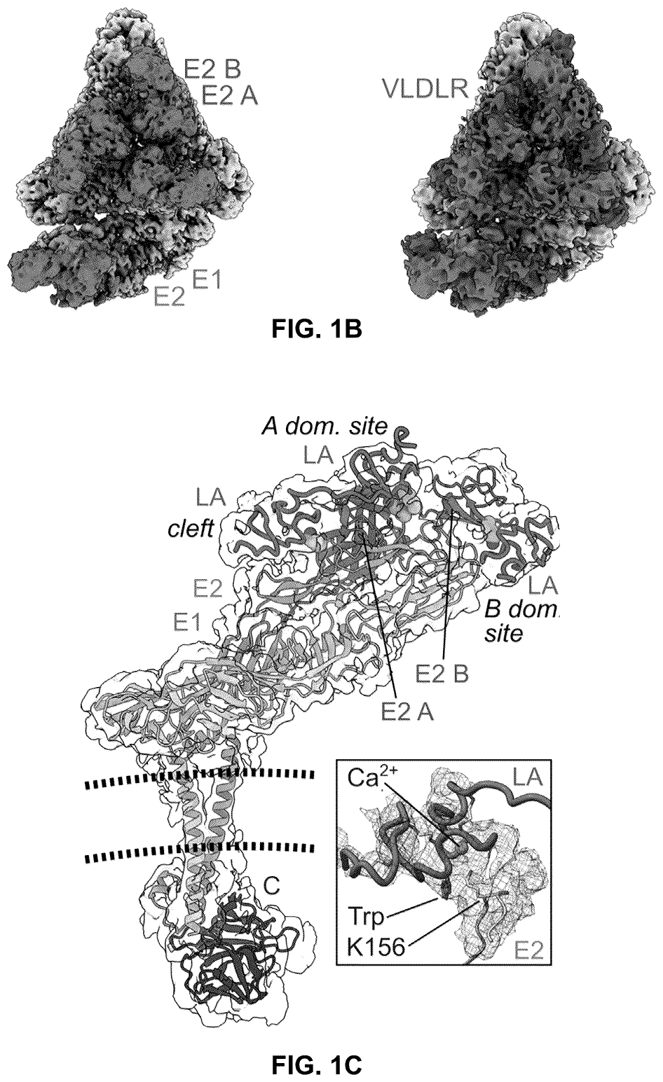

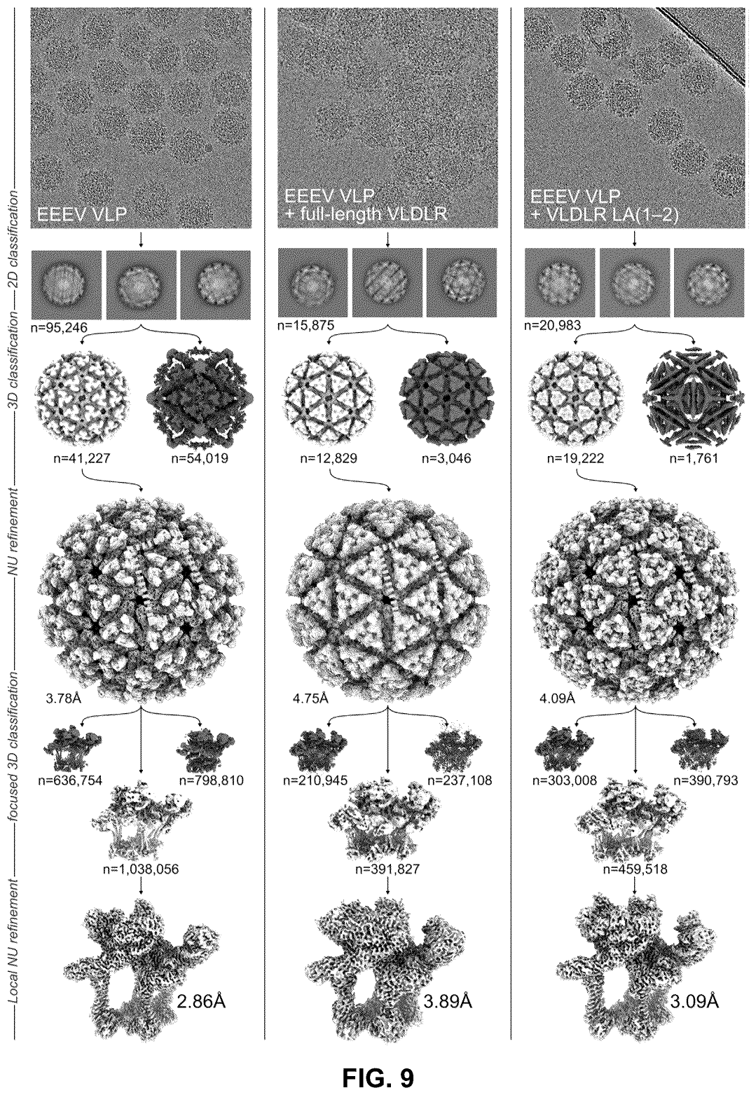

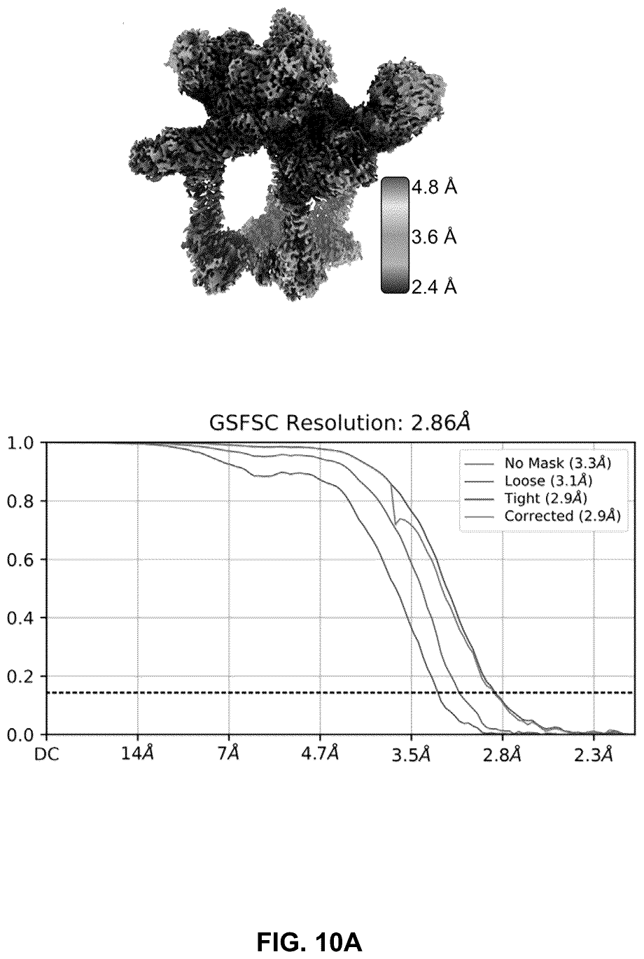

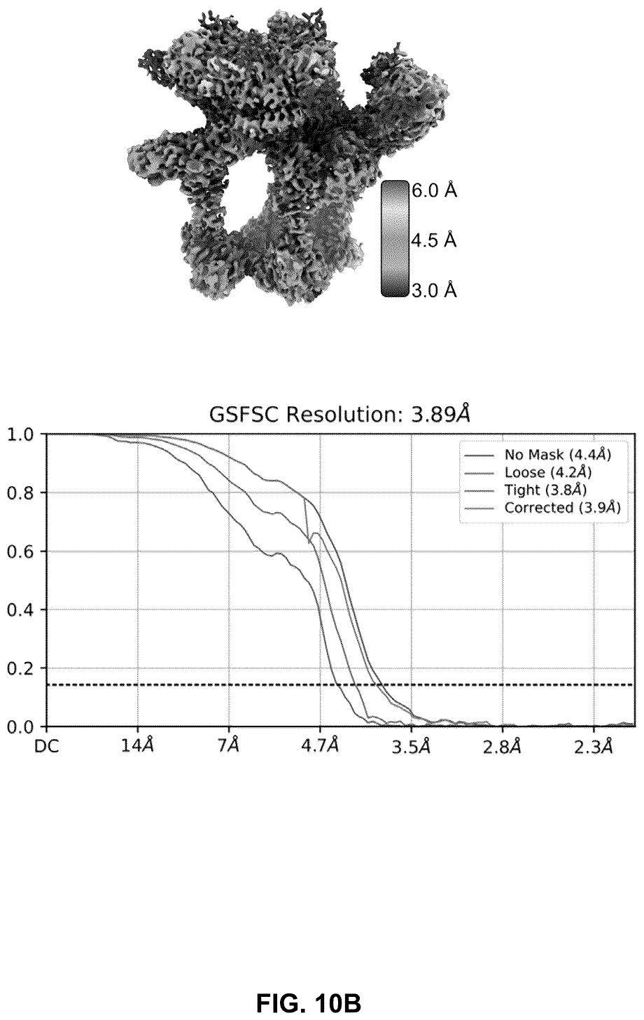

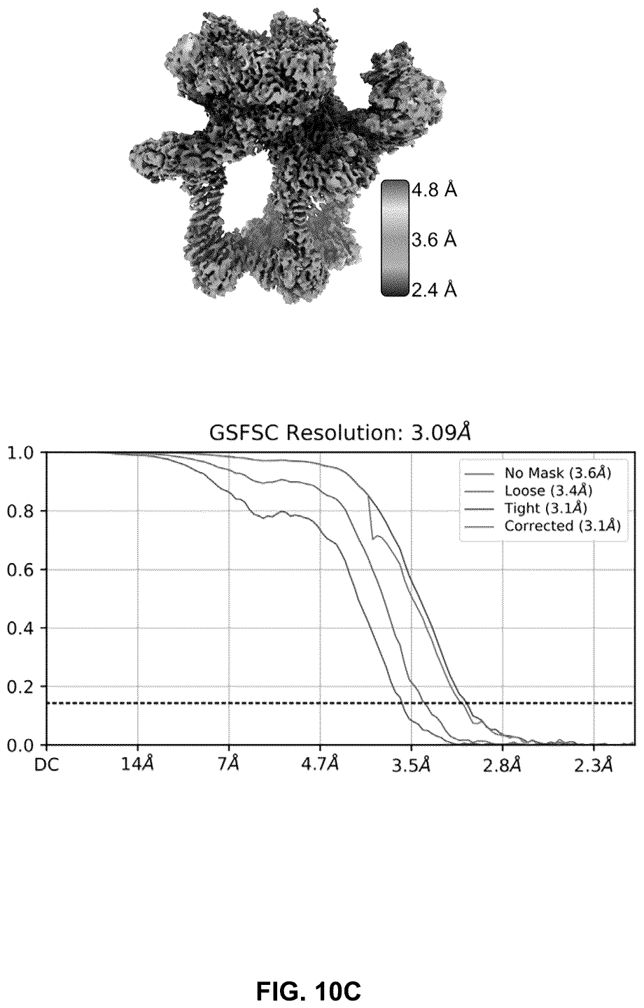

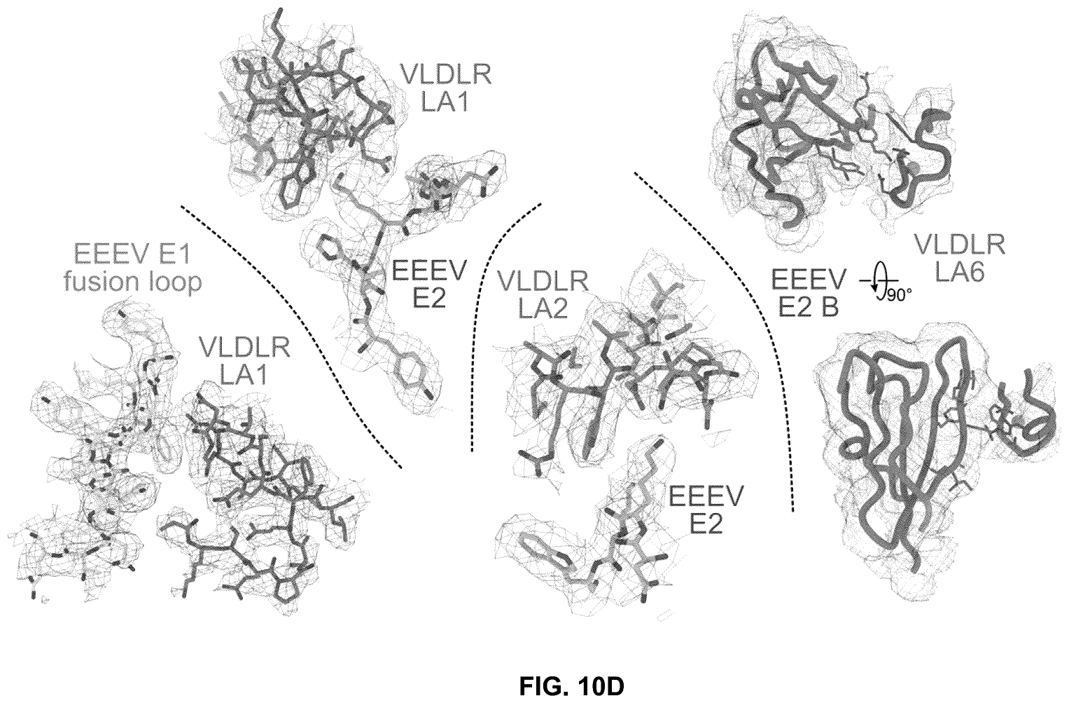

The ligand binding ectodomain (LBD) of VLDLR features eight cysteine-rich LDLR class A (LA) repeats and is necessary and sufficient to mediate infection by EEEV. To elucidate the structural basis for recognition of VLDLR by EEEV, EEEV virus-like particles (VLPs) (strain PE-6) were reconstructed alone or in complex with full-length VLDLR via cryo-EM (FIG. 9, FIG. 10A, FIG. 10B, Table 1), achieving respective resolutions of 3.78 Å and 4.75 Å with icosahedral symmetry imposed. As expected, the EEEV VLP exhibited T=4 icosahedral symmetry with 80 trimeric spikes (FIG. 1A, top). Additional densities consistent in size with LA repeats were evident in the VLDLR-bound structure (FIG. 1A, bottom). To orient the VLDLR LA domains within these densities, focused refinement of the asymmetric unit (ASU) was performed, generating apo and bound structures at 2.86 and 3.89 Å resolution, respectively (FIG. 1B).

TABLE 1
Cryo-EM data collection, processing, and model refinement statistics

| | Sample | | | | | | | | | |
|---|---|---|---|---|---|---|---|---|---|---|
| | | | | | | | EEEV | | | |
| | | | | | | EEEV | VLP | | | |
| | | EEEV | | | EEEV | VLP | PE-6 + | | EEEV | EEEV |
| | | VLP | EEEV | EEEV | VLP | PE-6 + | VLDLR | EEEV | VLP | VLP |
| | | PE-6 + | VLP | VLP | PE-6 + | VLDLR | LBD | VLP | PE-6 + | FL93- |
| | | full- | PE-6 + | PE-6 + | VLDLR | LA(1-6) | W132A | PE-6 + | VLDLR | 939 + |
| | EEEV | length | VLDLR | VLDLR | LA(1-5) | W132A | W210A | VLDLR | LA(1-6) | VLDLR |
| | VLP | VLDLR | LA(1-2) | LA(1-3) | W132A | W210A | W256A | LA(3-8) | W89A | LA(1-6) |
| EMDB | VLP: | VLP: | VLP: | VLP: | VLP: | VLP: | VLP: | VLP: | VLP: | VLP: |
| | 42187 | 42189 | 42191 | 42193 | 42195 | 42197 | 42199 | 42201 | 42202 | 42212 |
| | ASU: | ASU: | ASU: | ASU: | ASU: | ASU: | ASU: | | ASU: | |
| | 42188 | 42190 | 42192 | 42194 | 42196 | 42198 | 42200 | | 42203 | |
| PDB | ASU: | ASU: | ASU: | | | | | | | |
| | 8UFA | 8UFB | 8UFC | | | | | | | |

Data collection

| | | | | | | | | | | |
|---|---|---|---|---|---|---|---|---|---|---|
| Voltage (kV) | 300 | 300 | 300 | 300 | 300 | 300 | 200 | 300 | 300 | 300 |
| Magnification | 59,000× | 59,000× | 59,000× | 59,000× | 59,000× | 59,000× | 120,000× | 59,000× | 59,000× | 59,000× |
| Exposure (e−/Å2) | 37.93 | 37.90 | 37.90 | 37.90 | 44.40 | 44.40 | 39.90 | 44.57 | 44.40 | 44.13 |
| Defocus range (μm) | 0.7-2.2 | 0.7-2.2 | 0.7-2.2 | 0.7-2.2 | 0.7-2.2 | 0.7-2.2 | 0.5-1.9 | 0.7-2.2 | 0.7-2.2 | 0.7-2.2 |
| Pixel size (Å/pixel) | 1.081 | 1.081 | 1.081 | 1.081 | 1.081 | 1.081 | 1.184 | 1.081 | 1.081 | 1.081 |

Data processing

| | | | | | | | | | | |
|---|---|---|---|---|---|---|---|---|---|---|
| Final particles (no.) | VLP: | VLP: | VLP: | VLP: | VLP: | VLP: | VLP: | VLP: | VLP: | VLP: |
| | 41,227 | 13,998 | 19,222 | 19,321 | 1,465 | 4,724 | 2,813 | 13,331 | 1,057 | 9,669 |
| | ASU: | ASU: | ASU: | ASU: | ASU: | ASU: | ASU: | | ASU: | |
| | 1,038,056 | 391,827 | 459,518 | 491,574 | 55,592 | 144,008 | 84,264 | | 41,163 | |
| Resolution (Å) | VLP: | VLP: | VLP: | VLP: | VLP: | VLP: | VLP: | VLP: | VLP: | VLP: |
| | 3.78 | 4.75 | 4.09 | 4.21 | 4.94 | 4.05 | 6.03 | 3.66 | 5.72 | 4.72 |
| | ASU: | ASU: | ASU: | ASU: | ASU: | ASU: | ASU: | | ASU: | |
| | 2.86 | 3.89 | 3.09 | 3.24 | 3.71 | 2.98 | 4.75 | | 4.26 | |
| FSC threshold | 0.143 | 0.143 | 0.143 | 0.143 | 0.143 | 0.143 | 0.143 | 0.143 | 0.143 | 0.143 |

Model refinement

| | | | |
|---|---|---|---|
| Initial models | 6XO4 | 6XO4 | 6XO4 |
| Model resolution (Å) | 2.8/3.1 | 3.8/4.3 | 3.0/3.4 |
| FSC threshold | 0.143/0.5 | 0.134/0.5 | 0.143/0.5 |
| Model composition | | | |
| Non-hydrogen atoms | 31,260 | 34,660 | 33,624 |
| Residues | 4,024 | 4,476 | 4,336 |
| Ligands (glycans) | 8 | 8 | 8 |
| Bonds (RMSD) | | | |
| length (Å) | 0.003 | 0.003 | 0.002 |
| Angles (°) | 0.679 | 0.566 | 0.648 |
| Validation | | | |
| Molprobity score | 0.83 | 1.04 | 1.00 |
| Clash score | 1.16 | 1.76 | 1.78 |
| Rotamer outliers (%) | 0.00 | 0.00 | 0.00 |
| Ramachandran | | | |
| Favored (%) | 98.05 | 97.45 | 97.70 |
| Allowed (%) | 1.95 | 2.55 | 2.30 |
| Outliers (%) | 0.00 | 0.00 | 0.00 |

The ASU of the EEEV-VLDLR complex featured connected densities in the cleft formed between E1/E2 heterodimers and on the slanted shelf of the E2 A domain, as well as a third, lower-resolution density on the outward-facing side of the E2 B domain and E1 domain II. Although this structure did not allow unambiguous assignment of specific LA domains, these LA domains were oriented clearly with conserved calcium-coordination sites proximal to residues K156 (cleft), K231/K232 (A domain), and K206 (B domain) of EEEV E2 (FIG. 10C). This reconstruction suggests a binding strategy analogous to that observed for SFV and VLDLR, in that basic residues (K345 and K347 of SFV E1) engage a conserved aromatic residue (e.g., tryptophan [Trp]) and negatively-charged, calcium-coordinating residues of the LA domain. In contrast to SFV, which engages VLDLR via a single site on E1 domain III, the structure disclosed herein suggests that EEEV uses three distinct sites on the viral glycoproteins to mediate binding. LA domain densities were not seen near domain III of EEEV E1.

### Mapping of LA Domains Recognized by EEEV.

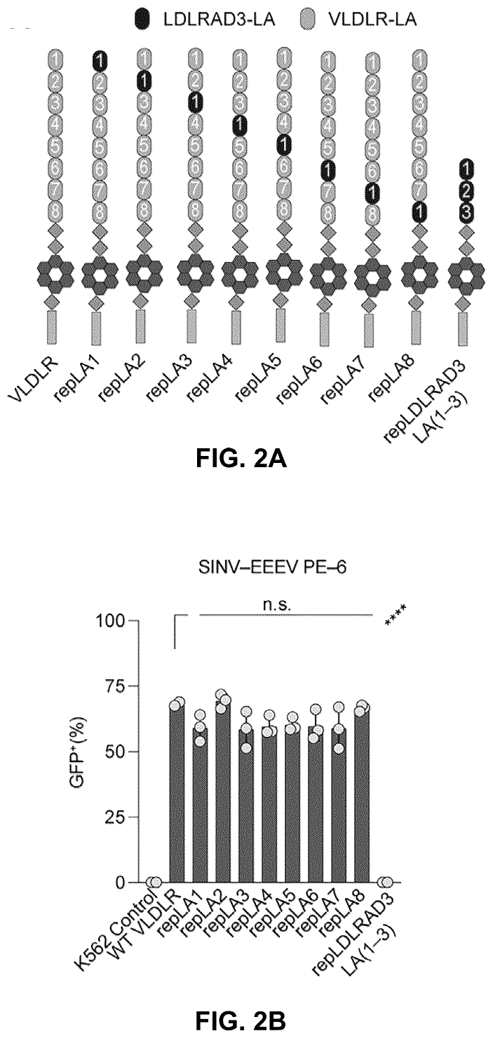

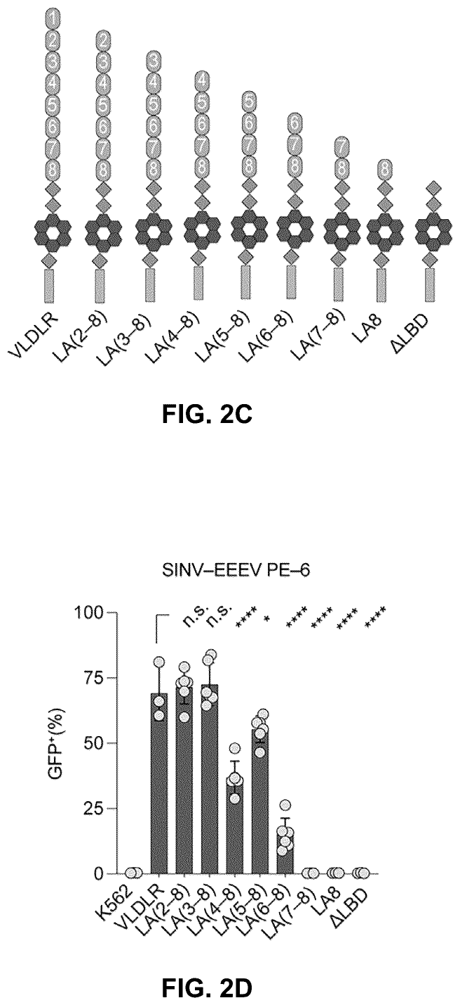

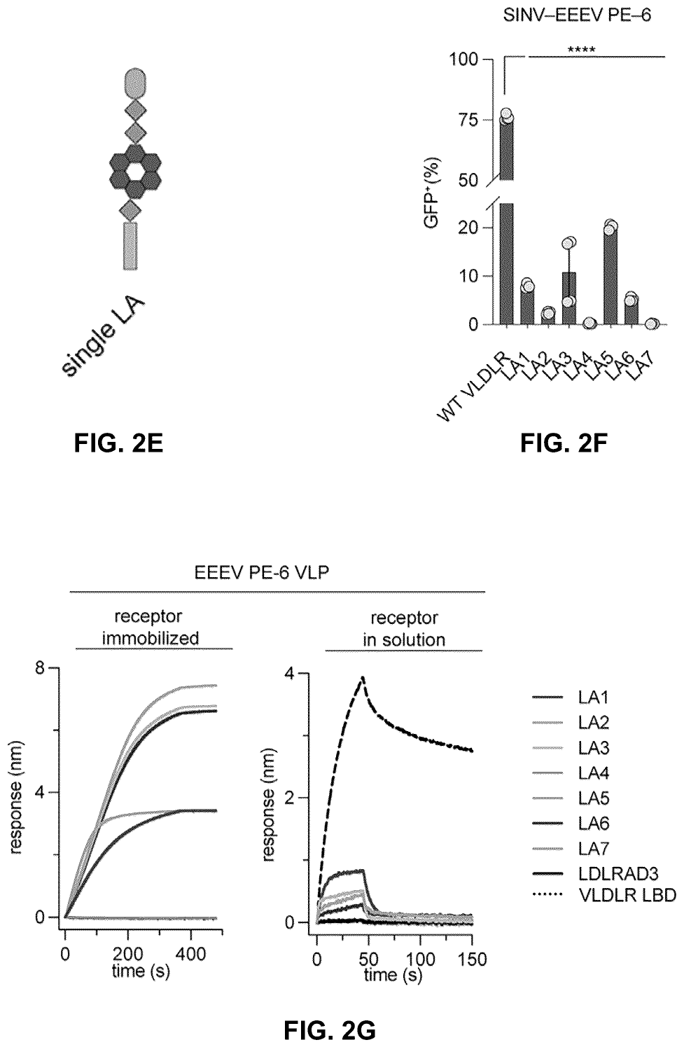

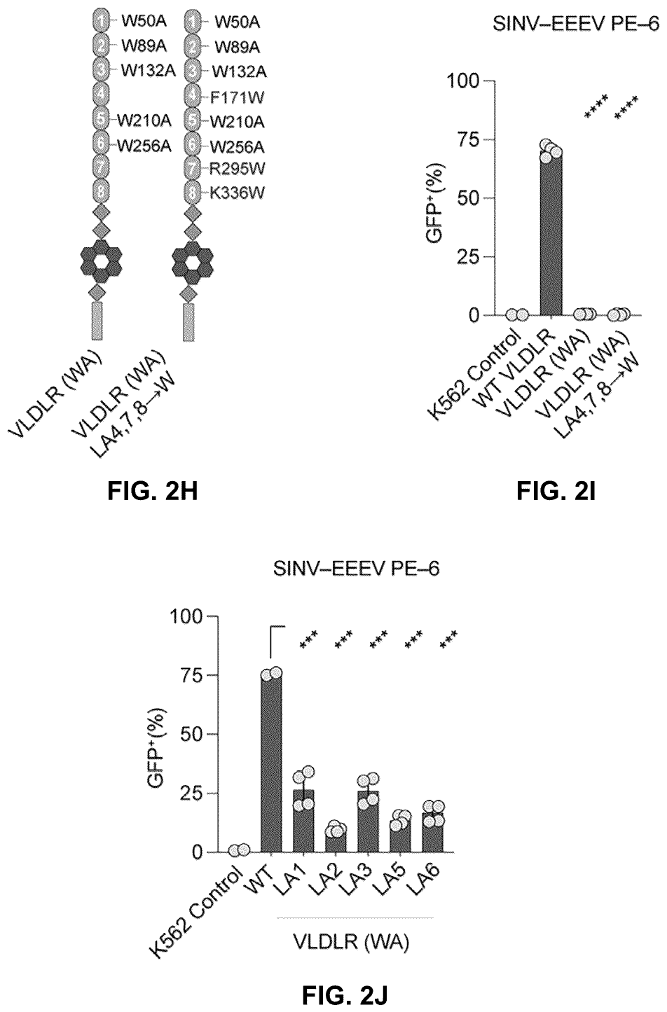

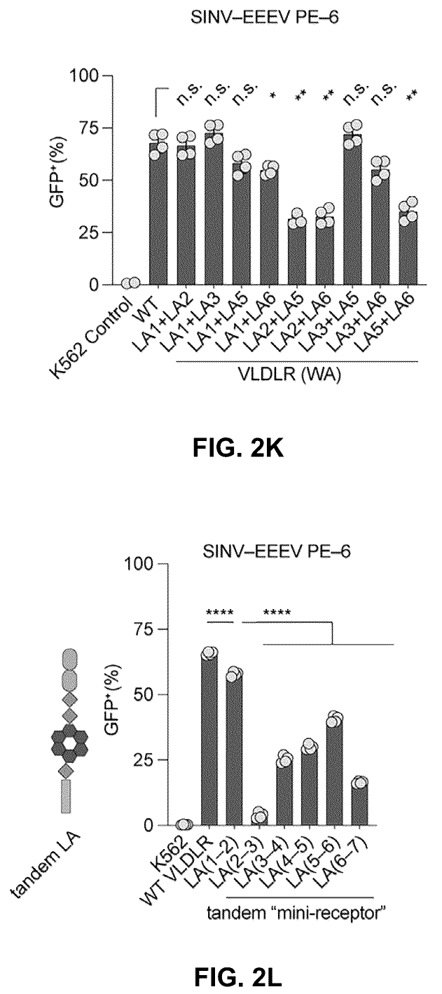

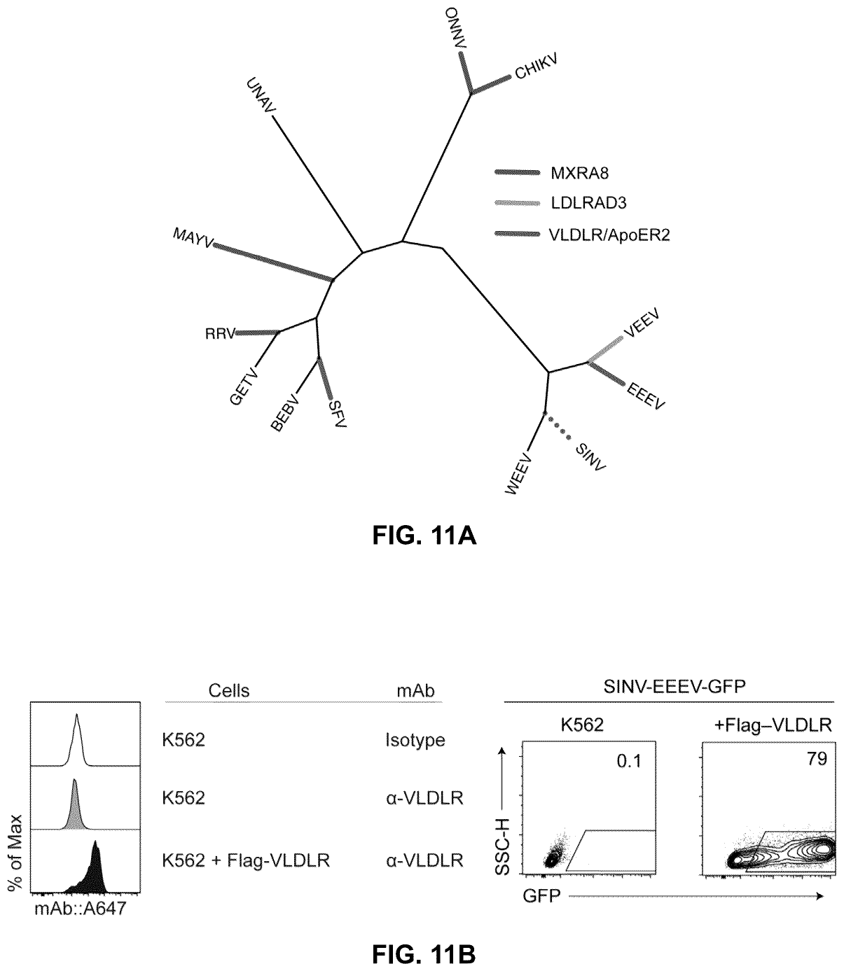

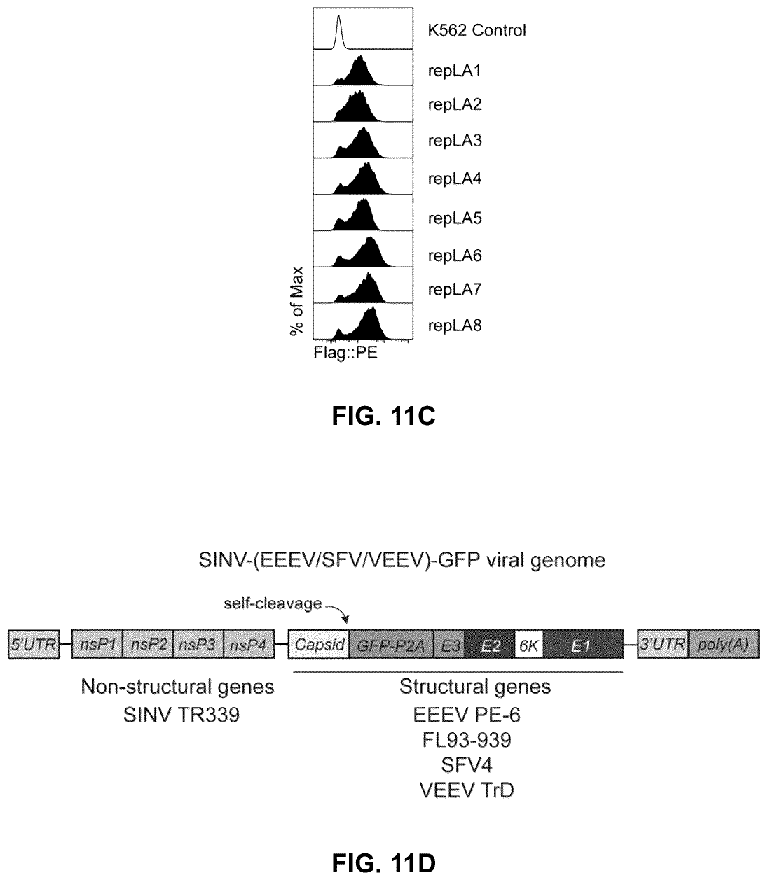

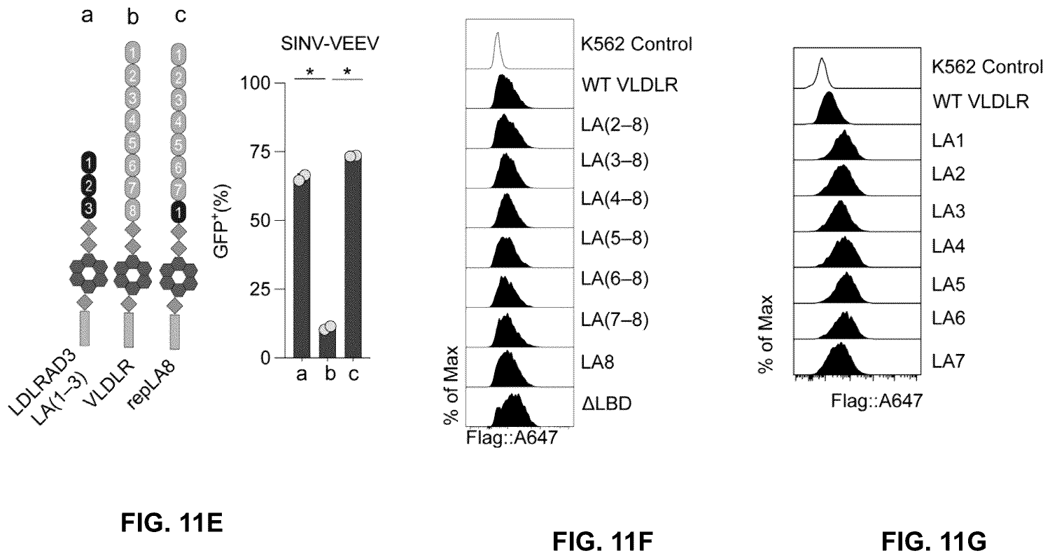

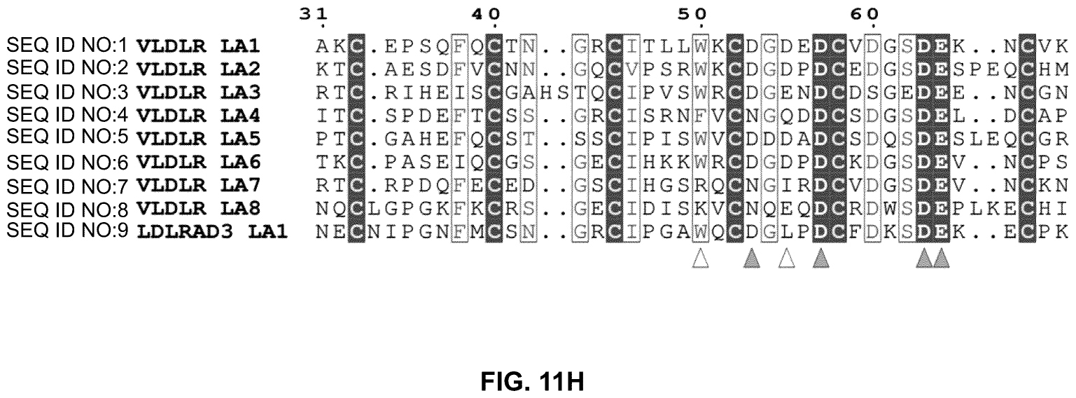

To assess the role of each LA domain in mediating EEEV infection, domain-swapped variants were generated of VLDLR with the LA1 domain of the related molecule, LDLRAD3, a receptor for VEEV (FIG. 2A, FIG. 11A) based on an approach used to map receptor domain usage of adeno-associated virus. Importantly, the LA1 domain of LDLRAD3 does not support infection by EEEV. K562 cells, which lack endogenous VLDLR expression and are non-permissive to EEEV (FIG. 11B), were transduced with N-terminally FLAG-tagged constructs in which a single LA repeat of VLDLR was replaced with LDLRAD3-LA1 and then confirmed cell surface expression by flow cytometry (FIG. 11C). To assess infection at a lower biosafety containment level, a chimeric Sindbis virus (SINV) GFP reporter virus was used, in which the structural genes of SINV are replaced with those of EEEV (SINV-EEEV; PE-6 strain) (FIG. 11D). Unexpectedly, infection was supported by all chimeric constructs, suggesting functional redundancy of at least some VLDLR LA domains (FIG. 2B). As an additional control, the LBD of VLDLR were replaced with LDLRAD3 (LA1-LA3); this chimera did not support infection of K562 cells by EEEV (FIG. 2B) but did promote VEEV infection (FIG. 11E). These results suggest multiple LA domains can bind to EEEV in a redundant fashion and/or no single site is necessary to support infection.

Next tested were N-terminally truncated LA domain variants of VLDLR on K562 cells for their ability to support SINV-EEEV PE-6 infection. After confirming equivalent cell surface expression of the different proteins (FIG. 2C, FIG. 11F), it was observed that LA(2-8) or LA(3-8) supported SINV-EEEV PE-6 infection at levels similar to parental VLDLR, whereas partial reductions of infection were seen with cells expressing LA(4-8) or LA(5-8) (FIG. 2D). However, K562 cells expressing LA(6-8) showed substantially reduced SINV-EEEV infection, and those expressing LA(7-8), LA8, and ΔLBD failed to support infection (FIG. 2D). To identify the LA domains that are functionally relevant for EEEV infectivity, individual LA domains were expressed in the context of the VLDLR ΔLBD backbone with an N-terminal FLAG-tag (FIG. 2E, FIG. 11H). After transducing cells, they were sorted for similar levels of FLAG-expression of all constructs relative to parental VLDLR (FIG. 11G) and then inoculated them with SINV-EEEV. Expression of LA1, LA2, LA3, LA5, or LA6 on the cell surface promoted infection, albeit less efficiently than parental VLDLR, whereas LA4, LA7, and LA8 did not support SINV-EEEV PE-6 infection above background (FIG. 2D, FIG. 2F). These results demonstrate that no single LA domain can support infection as efficiently as the parental 8 LA-domain containing protein, suggesting an advantage for using multiple LA domains as observed in structural data disclosed herein.

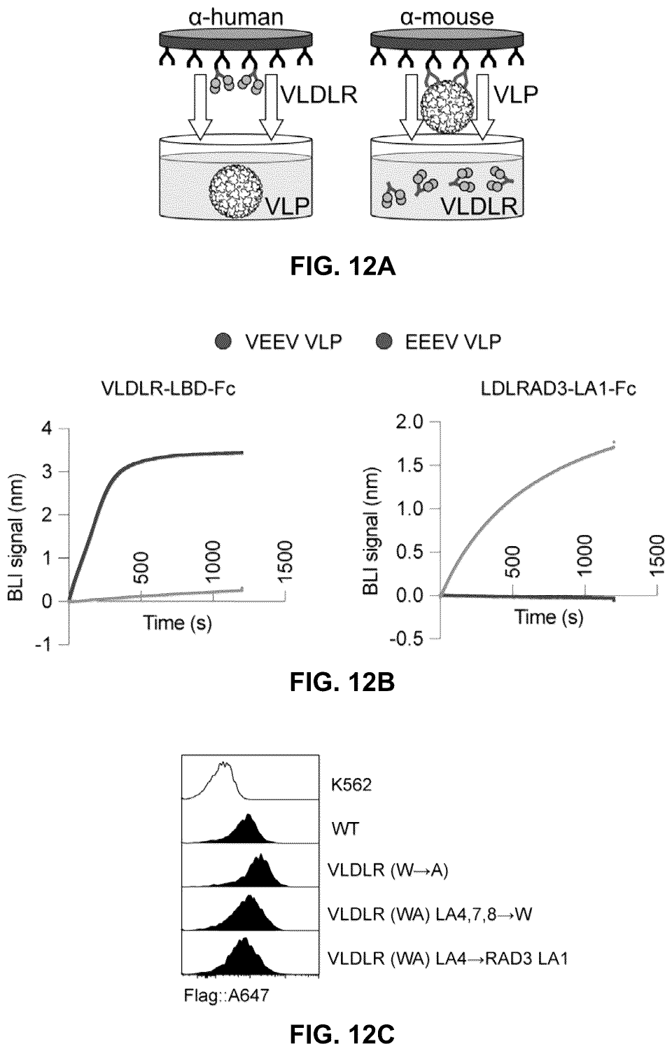

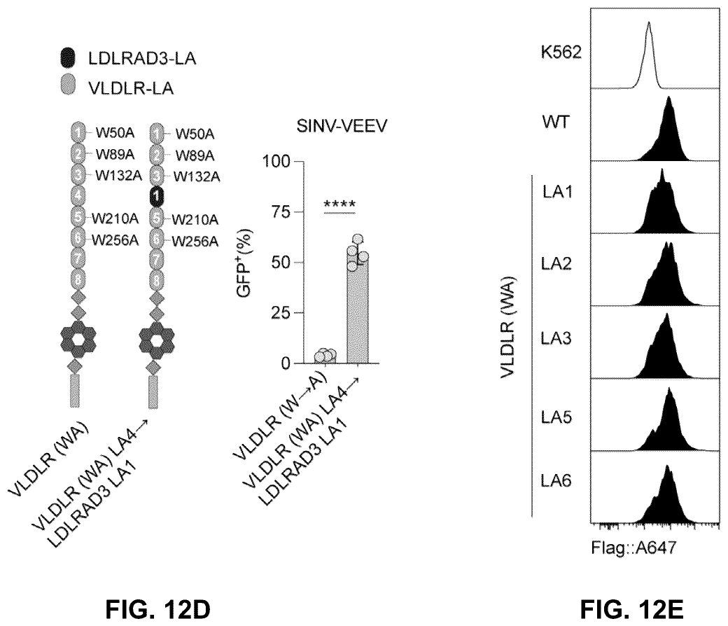

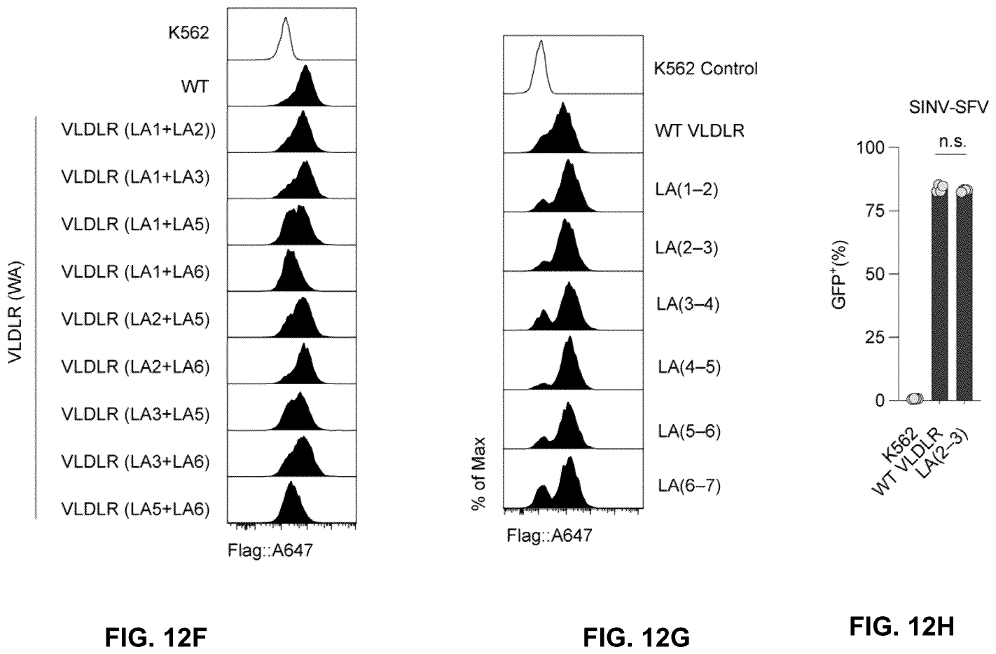

To corroborate these experiments, single LA-domain Fc fusion proteins were generated and evaluated them for binding to EEEV VLPs using biolayer interferometry (BLI) (FIG. 12A). First, the specificity of the binding assay was validated by immobilizing VLDLR-LBD-Fc (FIG. 12B) or LDLRAD3-LA1-Fc on anti-human Fc biosensors. As expected, pins coated with VLDLR-LBD-Fc bound to EEEV, but not VEEV VLPs, and reciprocally, pins coated with LDLRAD3-LA1-Fc bound to VEEV, but not EEEV VLPs (FIG. 12B). Individual VLDLR LA domains were then tested for binding to EEEV. Immobilized VLDLR-LA1, VLDLR-LA2, VLDLR-LA3, VLDLR-LA5, and VLDLR-LA6 captured EEEV VLPs (FIG. 2G, left), consistent with the single domain infection experiments. However, EEEV VLPs were unable to bind VLDLR-LA4 or VLDLR-LA7, again consistent with the infection data. To determine how efficiently the single LA domains could bind EEEV VLPs, VLPs were immobilized on mAb-coated biosensors and then evaluated binding of Fc-fusion proteins in solution. In this context, the VLDLR LA domains must bind without the advantage of avidity interactions. In contrast to full-length VLDLR LBD, the single LA domains showed marginal binding to EEEV VLPs in solution (FIG. 2G, right).

### Domain Binding Requirements for Efficient EEEV Infection.

Data disclosed herein shows that EEEV can recognize LA1, LA2, LA3, LA5, and LA6 but not LA4, LA7, or LA8. Sequence alignment of the VLDLR LA domains revealed that the domains recognized by EEEV all share a conserved Trp at the calcium-coordination site, which contrasts with those not recognized by EEEV (LA4, Phe; LA7, Arg; and LA8, Lys) (FIG. 11H). Indeed, prior work showed the Trp residue is critical for LA domain binding of VLDLR to SFV, and the orientation of the LA domains in the structure disclosed herein suggests an analogous mode of engagement (FIG. 1C) with density attributable to Trp observed for the LA domain residing within the E1/E2 cleft (interfacing with K156 of EEEV E2) (FIG. 1C, inset). To test this, K562 cells expressing a construct were generated in which the Trp of LA1 (W50), LA2 (W89), LA3 (W132), LA5 (W210), and LA6 (W256) of full-length VLDLR were mutated (VLDLR (WA)), (FIG. 2H). VLDLR (WA) was unable to support EEEV infection (FIG. 2I, FIG. 12C), which confirms the functional importance of the Trp residues in these domains. Reciprocally, the corresponding residues of LA4, LA7, and LA8 were mutated to Trp in the context of VLDLR(WA). However, these proteins did not support EEEV infection (FIG. 2I), suggesting that other LA domain residues contribute to binding. To demonstrate that VLDLR(WA) could functionally support infection by an alphavirus, the LA4 domain was replaced with LDLRAD3 LA1, which enabled productive SINV-VEEV infection (FIG. 10w but detectable levels of EEEV infection was observed in cells expressing constructs in which only one LA domain encoded the conserved Trp (FIG. 2J, FIG. 12E). Given the presently disclosed structural data (FIG. 1C), the presence of two functional LA domains (re-inserted Trp residues) was next tested in the context of full-length VLDLR (WA) as to whether they would enhance infection. While some constructs (e.g., combination LA domains including LA2+LA5, LA2+LA6, and LA5+LA6) supported lower levels of infection, others (e.g., combination LA domains including LA1+LA2, LA1+LA3, and LA3+LA5) mediated infection at levels similar to the parental VLDLR (FIG. 2K, FIG. 11F). The connected LA densities in the herein disclosed structure suggested that EEEV might engage two contiguous LA domains simultaneously. To evaluate this, “mini-receptors” were expressed harboring tandem LA domains [LA(1-2), LA(2-3), LA(3-4), LA(4-5), LA (5-6), and LA(6-7)] on the VLDLRΔLBD backbone (FIG. 2L, FIG. 12G). LA(1-2) ΔLBD supported similar levels of EEEV infection compared to parental VLDLR (FIG. 2L), much like the full-length VLDLR (WA) with LA1 and LA2 intact (FIG. 2K). Lower levels of infection were observed with the other tandem domain “mini-receptors” with LA(2-3)ΔLBD barely supporting EEEV (FIG. 2L). LA(2-3)ΔLBD was confirmed functional as it supported SINV-SFV infection (FIG. 12H). Coupled with binding data (FIG. 2G), these results suggest that while LA(2-3) can support attachment of EEEV, it inefficiently promotes viral entry. Overall, these experiments support the requirement of concurrent interactions with multiple LA domains to support efficient EEEV infection.

### LA Domain Decoy Receptors Neutralize EEEV Infection.

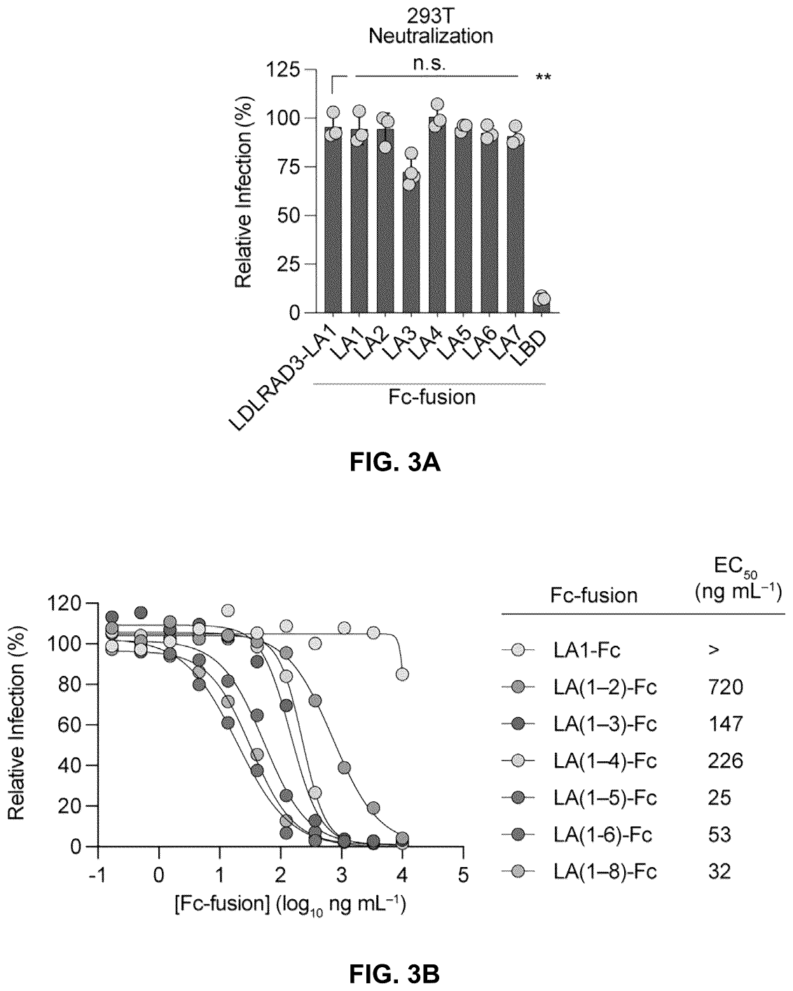

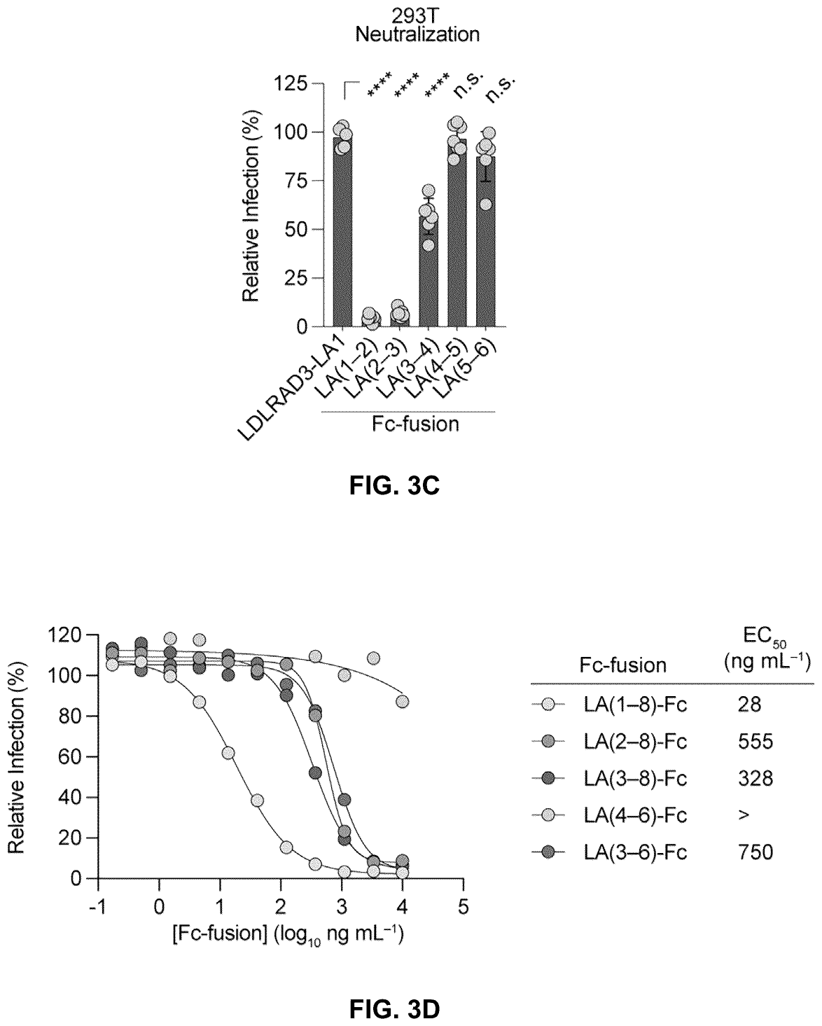

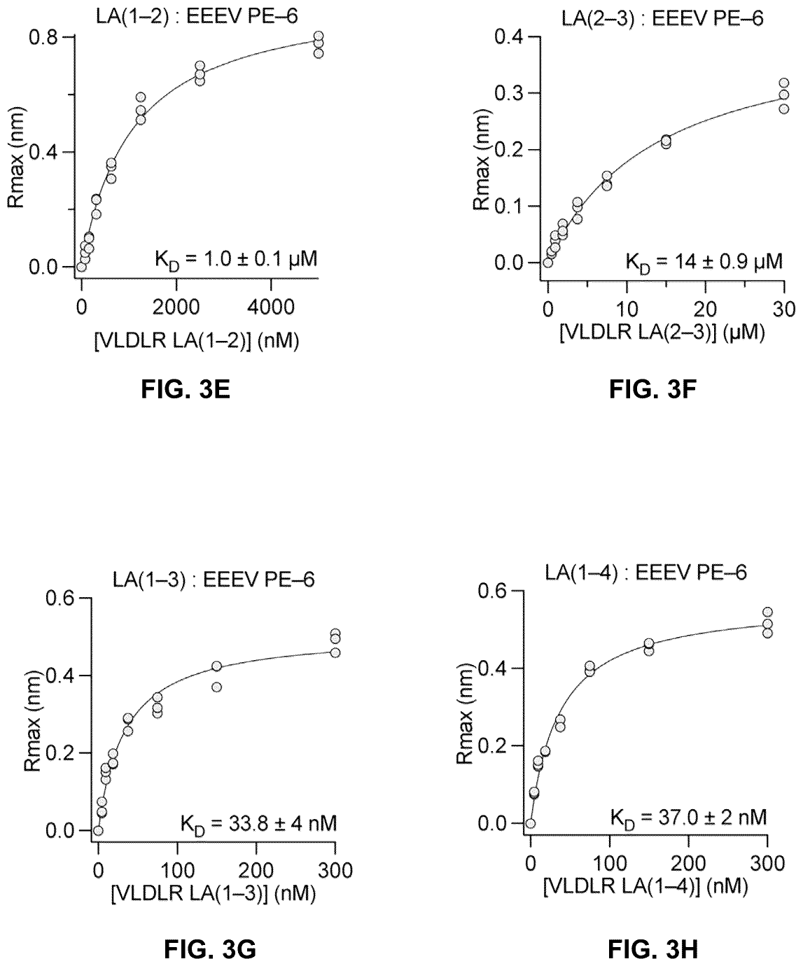

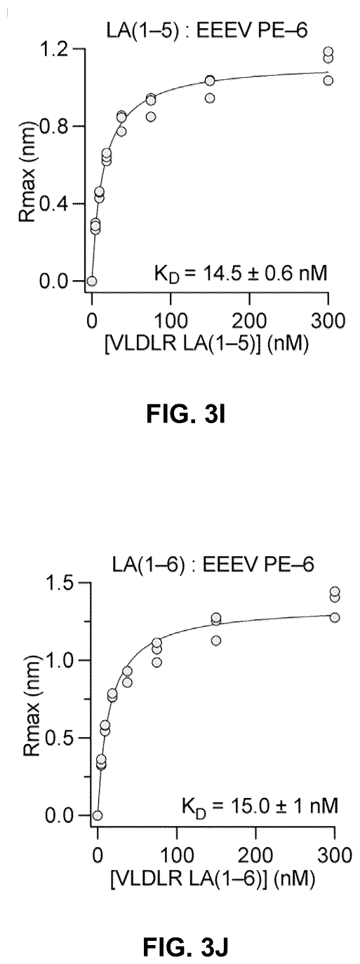

As another measure of how EEEV engages multiple LA domains, neutralization assays were performed with soluble decoy receptors comprised of different VLDLR LA domains. Fc-fusion proteins (10 μg/mL) were incubated with SINV-EEEV PE-6 prior to inoculation of 293T cells. Whereas VLDLR-LBD-Fc neutralized SINV-EEEV PE-6 almost completely, the single LA domain-Fc proteins did not (FIG. 3A), consistent with their poor binding in solution by BLI (FIG. 2G). Neutralization was then performed with different N-terminal fragments of the VLDLR receptor (LA(1-2), LA(1-3), LA(1-4), LA(1-5), and LA(1-6)) fused to an Fc domain. All N-terminal fragments neutralized infection, with increased potency observed with fragments that contain more LA domains (FIG. 3B). The results with LA(1-2)-Fc are consistent with the sufficiency of these two VLDLR LA domains to support efficient SINV-EEEV infection, although the potency of neutralization was greater for decoy proteins expressing additional LA domains that possibly could engage all three binding sites. This analysis was extended by testing additional two domain constructs (FIG. 3C). Whereas LA(1-2)-Fc and LA(2-3)-Fc completely neutralized infection at 10 μg/mL, LA(3-4)-Fc, LA(4-5)-Fc, and LA(5-6)-Fc did not. C-terminal fragments of VLDLR (LA(2-8)-Fc, LA(3-8)-Fc, LA(4-6)-Fc) were also tested. As LA(2-8)-Fc, LA(3-8)-Fc, and LA(3-6) efficiently inhibited SINV-EEEV PE-6 infection (FIG. 3D), binding and neutralization of EEEV PE-6 virions in solution does not require LA1 or LA2, consistent with efficient infection mediated by combinations of other domains (e.g., LA3+LA5) (FIG. 2K).

To better understand the functional hierarchy of LA domain engagement, steady-state affinity analyses were performed with monovalent forms of LA(1-2) and LA(2-3), as these molecules neutralized SINV-EEEV infection when expressed as Fc fusion proteins. Whereas LA(2-3) bound EEEV PE-6 VLPs with low affinity (K_{D}, 14±0.9 μM), LA(1-2) bound with 10-fold higher affinity (K_{D}, 1.0±0.1 μM) (FIG. 3E, FIG. 3F). The binding of LA(1-3), LA(1-4), LA(1-5), and LA(1-6) were then evaluated to determine whether the addition of more C-terminal LA domains would enhance affinity (FIG. 3G, FIG. 3H, FIG. 3I, FIG. 3J). Indeed, addition of LA3 improved binding, with LA(1-3) and LA(1-4) exhibiting K_{D} values of 33.8±4 and 37.0±2 nM, respectively. The enhancement of affinity plateaued with the addition of LA5 and LA6, as LA(1-5) and LA(1-6) bound with respective K_{D} values of 14.5±0.6 nM and 15.0±1 nM. Taken together, tandem LA domain neutralization (FIG. 3C), affinity measurements (FIG. 3E, FIG. 3F, FIG. 3G, FIG. 3H, FIG. 3I, FIG. 3J), and “mini-receptor” infection (FIG. 2L) studies indicate that while EEEV PE-6 can redundantly bind other LA domains, it engages VLDLR LA(1-2) domains particularly well.

Cryo-EM Structures of EEEV VLP in Complex with VLDLR Fragments.

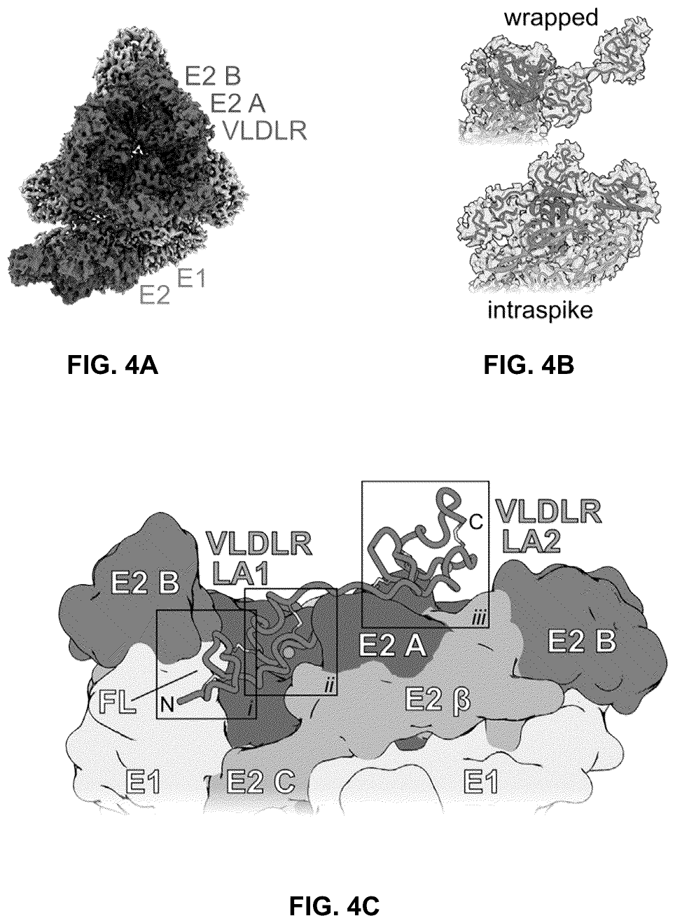

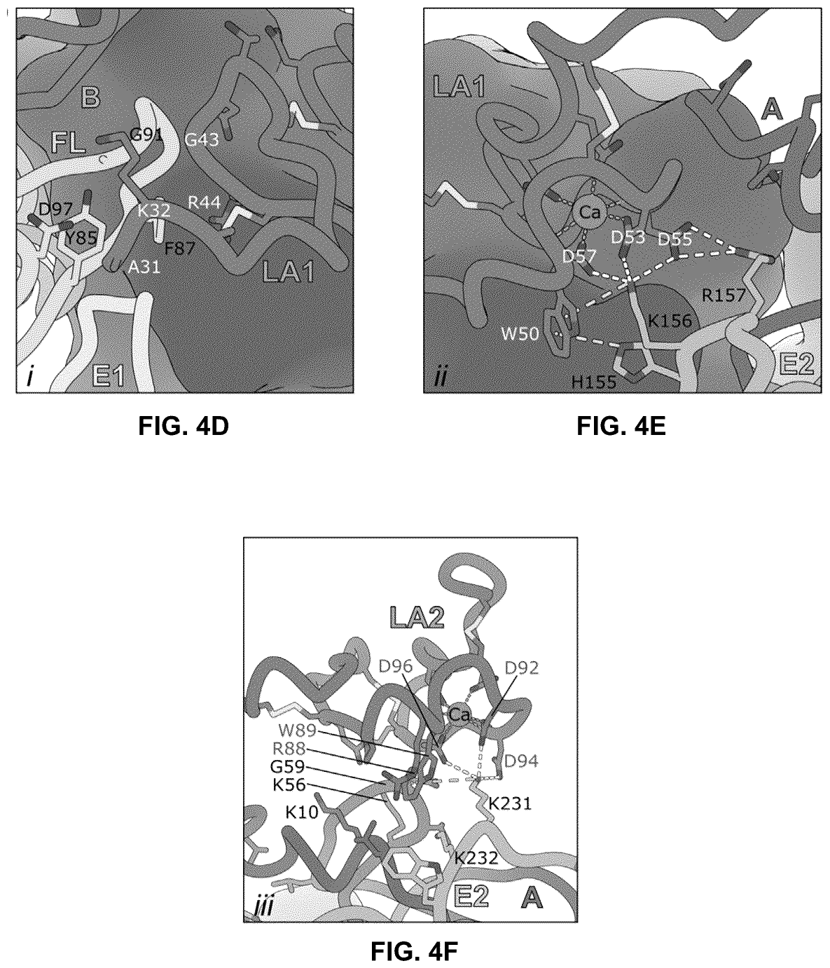

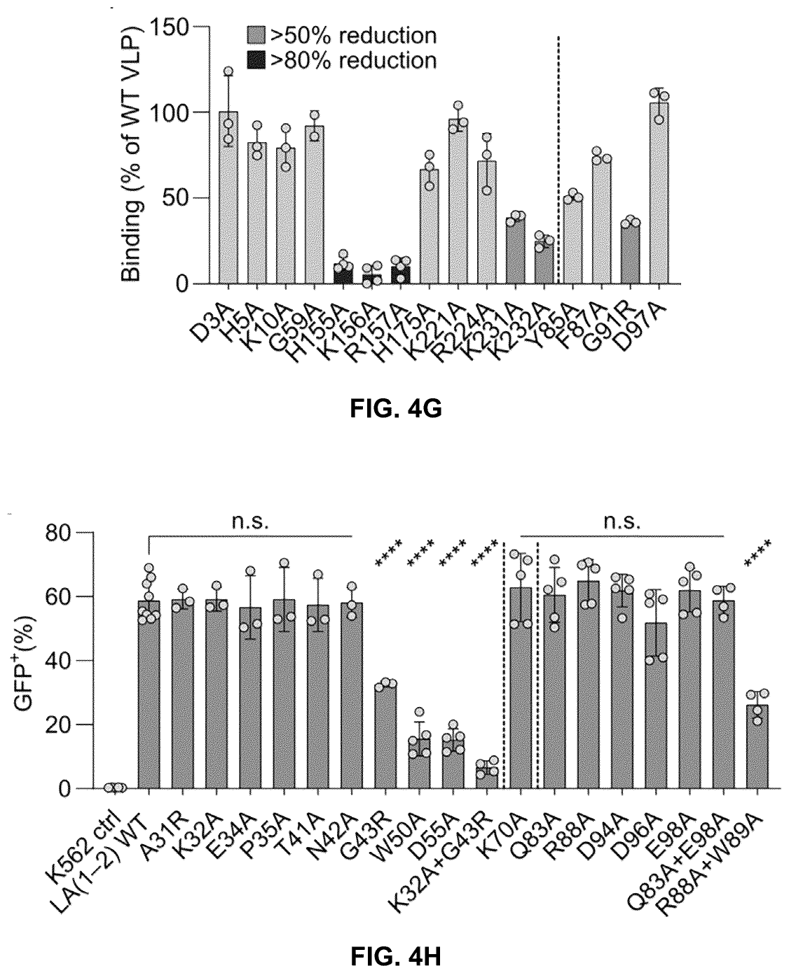

To understand how EEEV can engage different LA domains of VLDLR, it was sought to reconstruct EEEV VLPs in complex with different VLDLR fragments using cryo-EM. It was first assessed the structures of tandem sequential LA domains bound to EEEV VLPs, as a clear linking density was observed between the cleft and A domain site in the herein disclosed full-length VLDLR-EEEV structure. Focus centered on LA(1-2) and LA(2-3), as these proteins (as Fc-fusions) neutralized SINV-EEEV PE-6 infection, and also included LA(5-6) as both individual domains can interact with EEEV (FIG. 2F, FIG. 2G). For uncertain reasons, no receptor density was observed for LA(2-3) or LA(5-6), as either monomeric proteins or bivalent Fc fusions, despite incubation of EEEV VLPs with ten-fold molar excess of receptor in solution. However, when EEEV VLPs were incubated with monomeric VLDLR LA(1-2), two LA domains with a linking density were observed with high occupancy (FIG. 4A), matching the connected domains observed in the full-length VLDLR structure (FIG. 1C). To better define this interaction, focused refinement of the asymmetric unit to 3.09-Å resolution was performed (FIG. 4A, FIG. 9, FIG. 10C, Table 1, Table 2), enabling delineation of most sidechains at the binding interface (FIG. 10D). VLDLR LA1 binds within the cleft between E1/E2 heterodimers, establishing conventional “wrapped” and “intraspike” contacts to neighboring heterodimers (FIG. 4B, FIG. 4C), whereas LA2 assumes an angled position atop the A domain of EEEV E2, a site not occupied in other alphavirus-receptor structures (FIG. 4C). At the wrapped interface, VLDLR LA1 engages residues of the conserved fusion loop of EEEV E1 (residues 85, 87-92, 95, 97, and 98) (FIG. 4D). On the opposing face of the cleft, LA1 forms electrostatic interactions with residues H155-K156-R157 of the intraspike E2 (FIG. 4E). This electropositive “HKR” loop of EEEV E2 targets the highly conserved calcium-coordination site of LA1, establishing salt-bridges with VLDLR residues D53, D55, and D57, as well as hydrophobic and cation-π interactions with W50 (FIG. 4E). EEEV similarly targets the calcium cage of VLDLR LA2, coordinating via E2 residue K231 with VLDLR residues D92, D94, and D96, as well as W89; LA2 is also stabilized by K232 and an additional loop (residues 56-59) from the A domain of E2 (FIG. 4F).

To corroborate the herein disclosed structural model of EEEV in complex with LA(1-2), clones were selected from a previously generated VLP expression library⁴¹ (EEEV strain FL93-939) encoding alanine mutations in the E2 gene and measured binding to VLDLR LA(1-2)-Fc protein by BLI. Compared to wild-type EEEV VLPs, an almost complete loss of binding of VLDLR LA(1-2)-Fc was observed with EEEV VLPs encoding E2-H155A, E2-K156A, or E2-R157A substitutions, and a partial loss of binding was measured with E2-K231A or E2-K232A mutants (FIG. 4G). Several other E2 substitutions (D3A, H5A, K10A, G59A, H175A, K221A, R224A) did not impact EEEV VLP binding, suggesting a less critical role for these residues (FIG. 4G). To assess the interaction of the LA1 domain with the fusion loop in E1, additional mutant VLPs were generated, including E1-Y85A, E1-F87A, E1-G91R, and E1-D97A, and evaluated binding of LA(1-2)-Fc; greater than 50% (>50%) reduction was observed in binding for E1-G91R (FIG. 4G), likely due to steric hindrance at this site or reduced dihedral flexibility of the fusion loop.

TABLE 2
List of residues comprising EEEV/VLDLR interface.

| E1/E2 heterodimer | E1/E2 domain | E1/E2 residues | VLDLR residues |
|---|---|---|---|
| wrapped | E2 A domain | R26, C27, D28 | LA1: R44 |
| | E2 B domain | H175, V220, K221, | LA1: A31, K32, Q39, C40, T41, N42, |
| | | R224 | G43, R44, D60 |
| | E1 fusion loop | Y85, F87, M88, | LA1: A31, K32, Q39, N42, G43, R44, |
| | | Y89, G90, G91, | C45, V59, D60 |
| | | A92, F95, D97, T98 | |
| | E1 domain II | G228, I229 | LA1: R44 |
| intraspike | E2 A domain | D1, L2, D3, T4, H5 | LA1: C52, D53, G54, D55, E64, C67 |
| | | K10, L11, K56, T57, | Linker: V68, K69, K70 |
| | | D58, G59, V60, L62 | LA2: C72, Q83, C84, V85, P86, R88, |
| | | | W89, D94, P95, D96, C97, E98 |
| | E2 B domain | S191, G192, N230 | LA2: R88, D92, G93, D94 |
| | E2 β-linker | T154, H155, K156, | LA1: L49, W50, D53, D55, D57 |
| | | R157, A158, K231, | LA2: R88, W89, D92, D94, P95, D96 |
| | | K232, W233 | |
| PE-6 contacts | E2 B domain | Y197, Y198, K200, | LA6: E243, G249, E250, C251, H253, |
| | | P202, D203, V204, | K255, W256, D259, D261, P262, |
| | | R205, K206, G207, | D263, C264, K265 |
| | | I208 | |
| | E1 domain II | K61, C62, C63, G64 | LA6: D259, G260, D261, P262 |
| | E1 fusion loop | E99 | LA6: P262 |

To further evaluate the instant model, structure-guided mutants were generated in both LA1 and LA2 domains in the context of the LA(1-2)ΔLBD “mini-receptor” and assessed their effects on SINV-EEEV PE-6 infection. Of the 9 mutants made in the LA1 domain, only G43R, W50A, and D55A substitutions resulted in decreased SINV-EEEV PE-6 infection (FIG. 4H, FIG. 12I). A K32A/G43R double substitution showed greater reduction in infectivity, consistent with the LA1-E1 fusion loop interaction in the model and the impaired binding of LA(1-2)-Fc to E1-G91R (FIG. 4D, FIG. 4H). Although the single LA2 domain substitutions tested (K70A, Q83A, R88A, or D94A) did not impair the ability of LA(1-2)ΔLBD to support infection, loss of infectivity was observed with R88A+W89A in LA2, confirming the importance of the conserved Trp (FIG. 4H).

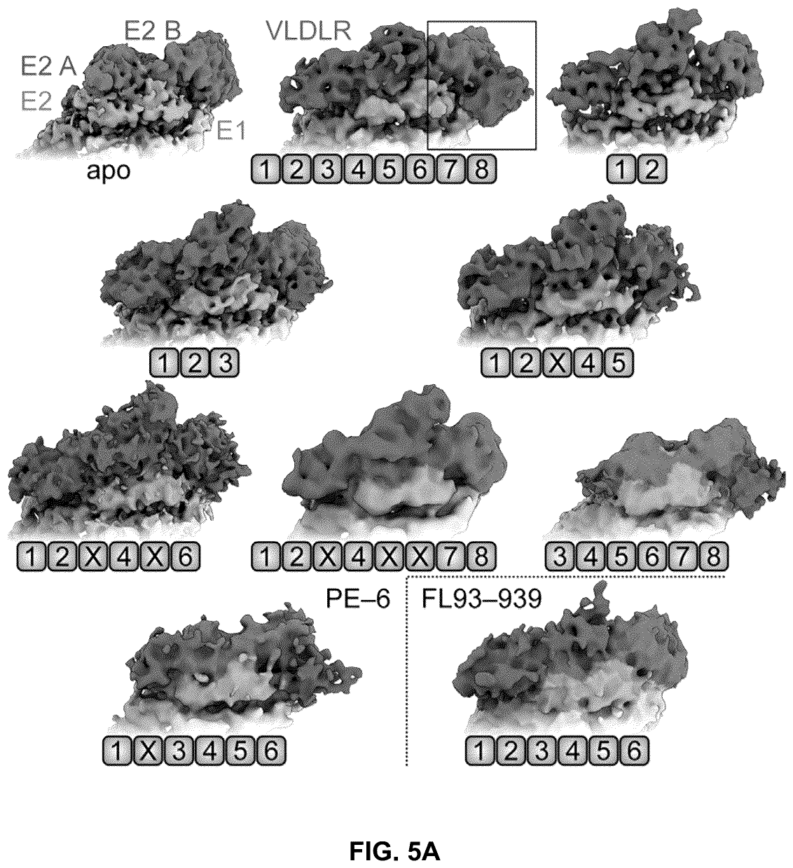

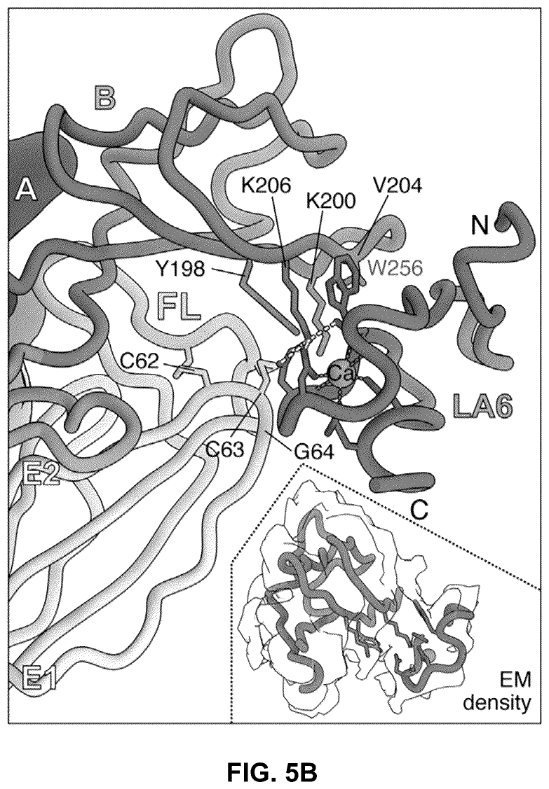

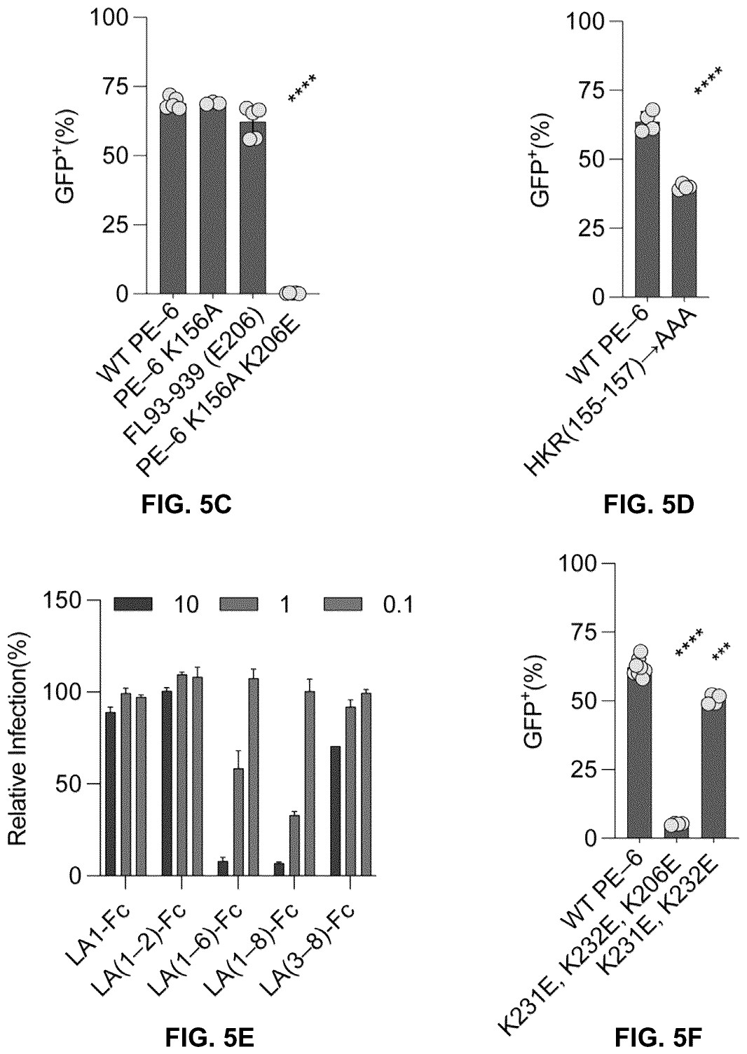

As visualization of LA(2-3) or LA(5-6) via cryo-EM was not achieved, it was characterized how EEEV engages LA3, LA5, and LA6 by complexing EEEV PE-6 VLPs with LA(1-3), LA(1-5mut3) (harboring a W132A mutation in LA3), or LA(1-6mut3,5) (harboring W132A and W210A mutations in LA3 and LA5, respectively), as LA(1-2) could anchor VLP binding to the E1/E2 cleft and E2 A domain sites (Table 1). Each of these reconstructions showed a density, albeit not fully occupied, at the lateral E2 B domain site (FIG. 5A), suggesting that LA3, LA5, or LA6 can engage this site interchangeably. To verify that the B domain density in the full-length VLDLR structure can be attributed to LA3, LA5, and LA6, EEEV VLPs were reconstructed in complex with VLDLR LBDmut3,5,6 (harboring W132A, W210A, and W256A in LA3, LA5, and LA6, respectively). As expected, only LA1 and LA2 domains with no visible density were observed at the lateral B domain site (FIG. 5A).

Since no single LA domain engaged the E2 B domain site with full occupancy, the B domain density produced by full-length VLDLR was used to build LA6 (as an example) for an interface analysis. In the instant model, LA6 principally engages E2-K206 via W256, D259, D261, and D263 within the calcium-coordination site (FIG. 5B). VLDLR may contact other residues of E2 (e.g., Y198, K200, and V204) or of E1 domain II (e.g., C62, C63, and G64) at this site; however, at a moderate contour threshold, only the calcium-coordination site is visible in the map disclosed herein (FIG. 5B, FIG. 10D). The minimal nature of this interface (<300 Å² buried surface area, with ˜120 Å2 solely on K206) may explain the LA domain promiscuity observed at this binding site as well as the relative difficulty of resolving a single domain at this position.

To further define the patterns of VLDLR domain binding to EEEV, cryo-EM reconstructions of EEEV VLPs were generated in complex with LA(3-8) or LA(1-6mut2) (harboring W89 Å in LA2) (Table 1). For both reconstructions, densities in the cleft between E1-E2 heterodimers and on the side of E2 B domain were observed, whereas density was absent in the E2 A domain (FIG. 5A). This result indicates the glycoprotein cleft of EEEV can be recognized by different LA domains (including non-LA1 domains), whereas the E2 A domain appears to exhibit greater specificity for LA2, as other domains are not visualized at this site. Thus, multiple sites on the EEEV glycoprotein (E2/E1 cleft, E2 A domain, E2 B domain) can recognize different VLDLR LA domains with some degree of promiscuity. Notwithstanding this point, results suggest that EEEV engages LA1 and LA2 efficiently at the cleft and A domain sites, as these are the only LA domains fully and unequivocally visualized by cryo-EM with either full-length or fragmented VLDLR. The relative importance of LA(1-2) is consistent with functional studies, as they are the only tandem LA domains that support efficient SINV-EEEV infection, neutralize EEEV infection as Fc fusions, and bind with moderate affinity as monovalent proteins.

### EEEV Strains do not Conserve all VLDLR-Binding Sites.

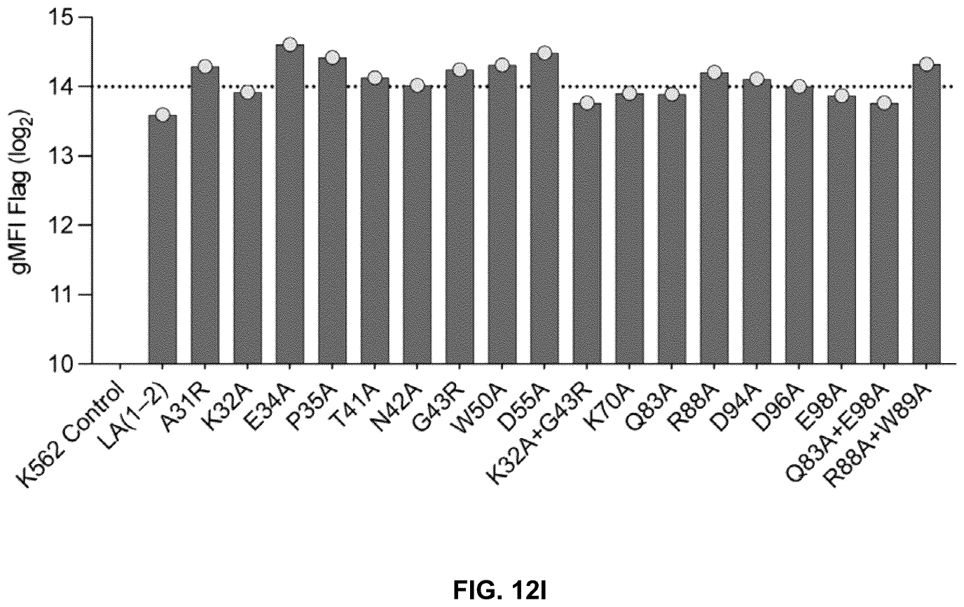

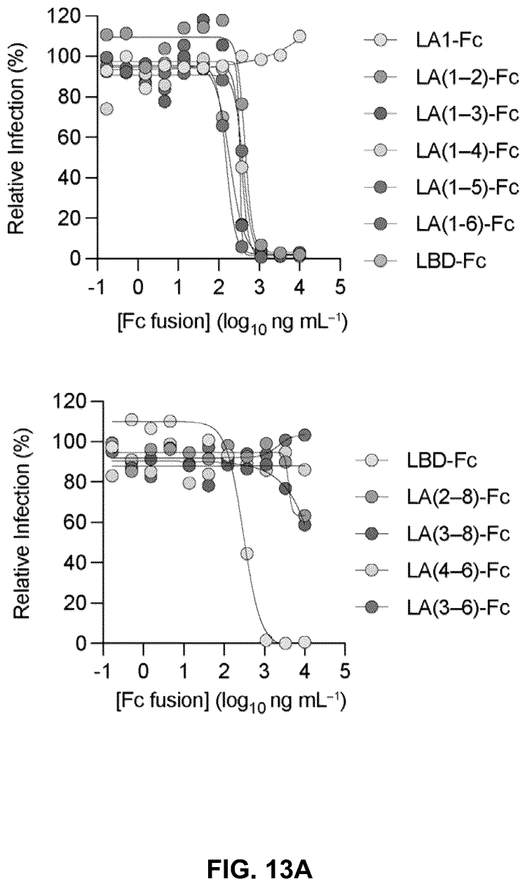

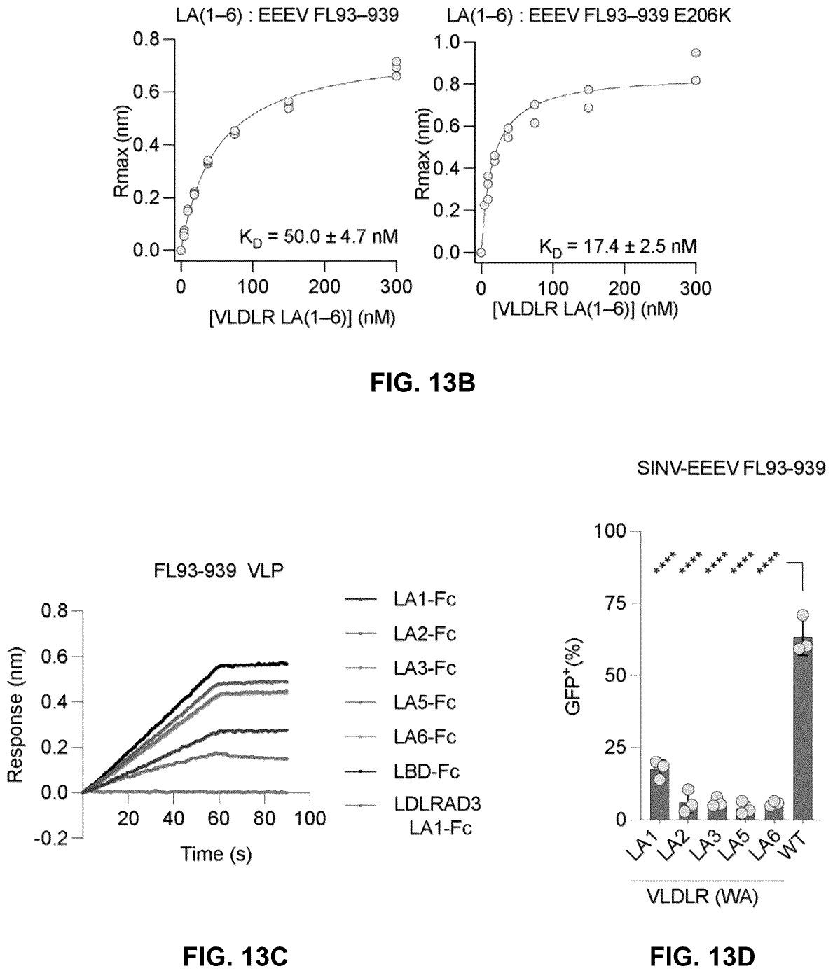

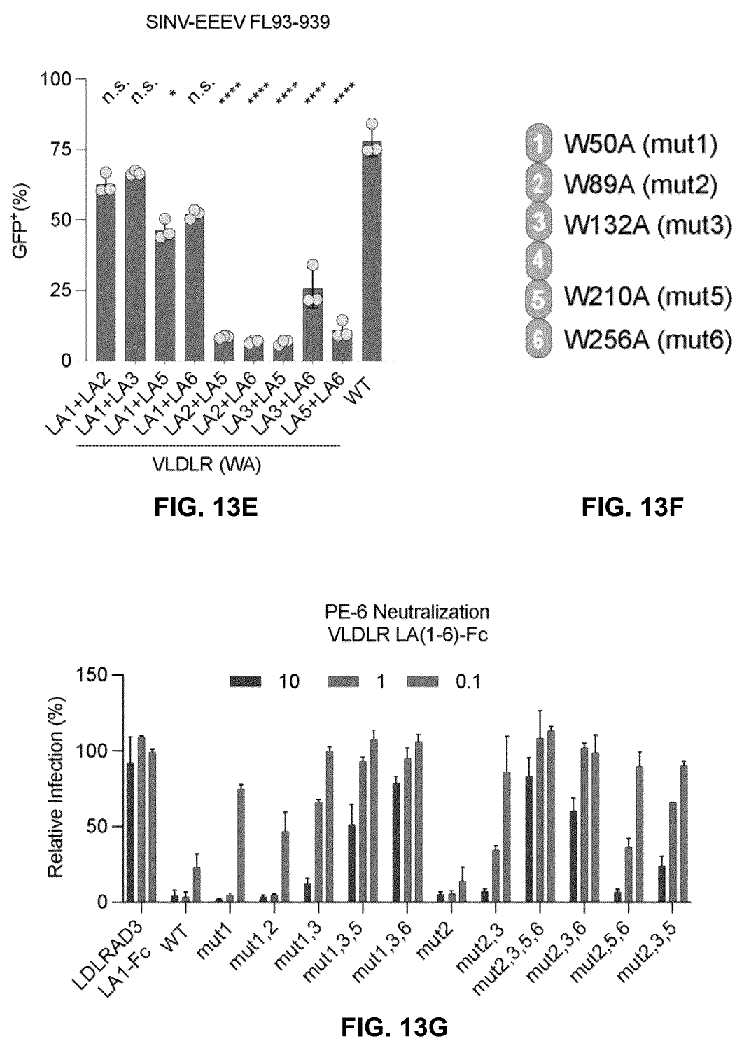

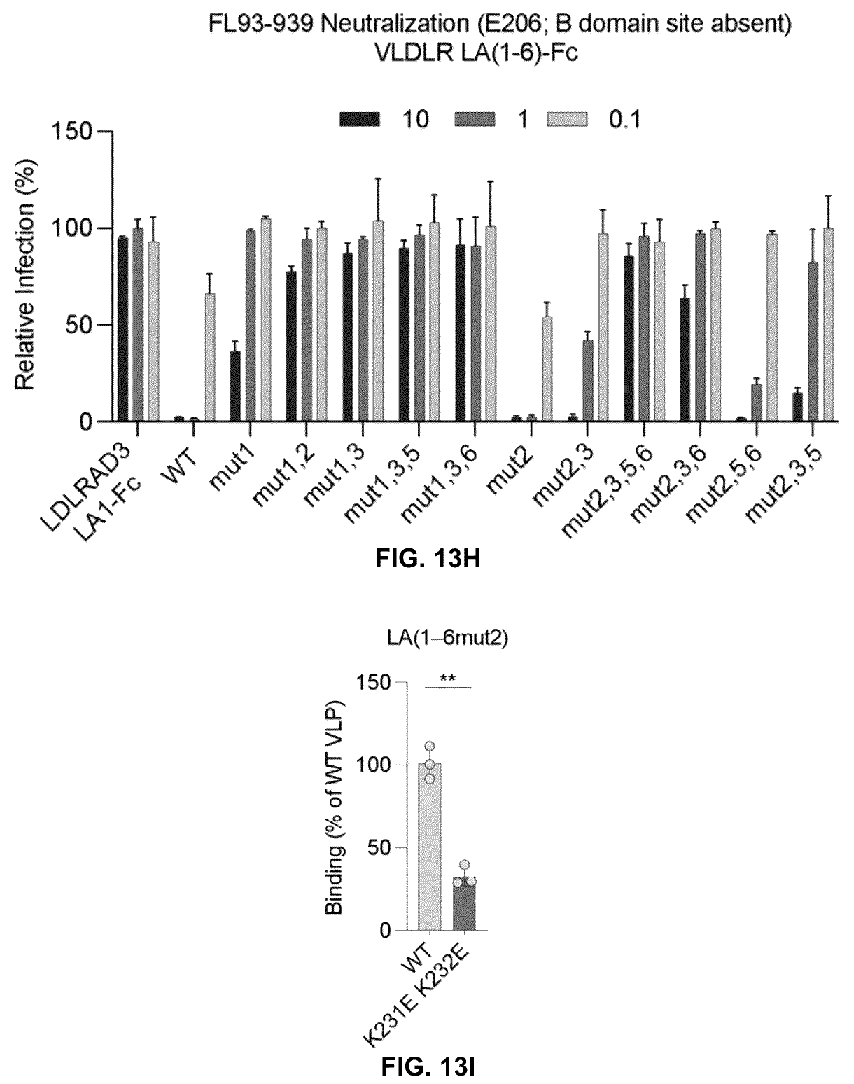

Next considered was whether there was redundancy in the three binding sites (E1/E2 cleft, E2 A domain, and E2 B domain), and that any given site might not be required to mediate VLDLR-dependent interactions. It was first evaluated whether the basic amino acids interacting with Trp of the LA domains were conserved in EEEV strains. The E2-HKR loop (H155-K156-R157; cleft) and E2-K/R231+E2-K232 (A domain shelf) residues are conserved in all 692 EEEV structural polyprotein sequences present in GenBank. However, the E2 B domain binding site, which features residue E2-K206 in the PE-6 strain, is not conserved in FL93-939 (E206) or 688 other EEEV strains; residue E2-K206 is present in only one other EEEV strain (A61-1K) and one EEE complex Madariaga virus strain (BeAr348998). To confirm that the E2-E206 substitution abrogates VLDLR engagement at the B domain site, cryo-EM was performed with VLDLR LA(1-6) complexed with EEEV FL93-939 VLPs. As expected, receptor density at the B domain site for FL93-939 was not observed (FIG. 5A). Moreover, VLDLR LA(1-2)-Fc neutralized SINV-EEEV FL93-939 infection equivalently compared to LA(1-3)-Fc, LA(1-4)-Fc, LA(1-5)-Fc, LA(1-6)-Fc, or LBD-Fc (FIG. 13A), providing supporting evidence that LA3, LA5, and LA6 do not engage a third site on FL93-939. Given that a prior study showed VLDLR-dependent infection with reporter virus particles encoding FL91-469 structural genes, the E2-K206 residue in EEEV PE-6 may be dispensable or act as a gain-of-function. As K562 cells expressing VLDLR are permissive to SINV-EEEV FL93-939 (FIG. 5C), engagement of the E2 B domain site on EEEV is not required for infectivity.

The E2-206K residue might provide an advantage for EEEV PE-6 by enhancing its binding for VLDLR. To test this possibility, affinity measurements of LA(1-6) were performed in complex with FL93-939 or FL93-939 engineered with an E2-E206K substitution. A slightly lower affinity of LA(1-6) binding was observed for FL93-939 (K_{D}, 50.0±4.7 nM) compared to PE-6 (15.0±1 nM), but this difference was ameliorated by the E2-E206K change in FL93-939 (K_{D}, 17.4±2.5 nM). Thus, EEEV PE-6 has a small but measurable advantage in VLDLR-binding that is due to the charge reversal at E2-206 (FIG. 13B).

The E2-E206K substitution might enable more versatile domain usage, as other LA domains (e.g., LA3, LA5, and LA6) could be engaged through the B domain binding site. To begin to address this possibility, it was evaluated whether LA3, LA5, and/or LA6 could bind and support infection of EEEV FL93-939. When immobilized on a biosensor in the solid phase, the single LA domain-Fc proteins could capture FL93-939 VLPs (FIG. 13C), and K562 cells transduced with VLDLR (WA) constructs with intact LA3, LA5, or LA6 domains supported low levels of SINV-EEEV FL93-939 infection (FIG. 13D). When VLDLR(WA) constructs were expressed on the cell surface with two intact LA domains (e.g., LA1+LA3, LA1+LA5, or LA1+LA6), more efficient SINV-EEEV FL93-939 infection was observed, comparable to that seen with LA1+LA2 (FIG. 13E); however, and in contrast to SINV-EEEV PE-6, domain combinations without LA1 did not support robust SINV-EEEV FL93-939 infection. The greater dependence of FL93-939 on VLDLR LA1 suggests that while non-LA1 domains can enter the E1/E2 cleft (as observed in the LA(3-8) reconstruction (FIG. 5A)), they cannot support efficient infection without assistance of the B domain VLDLR-binding site. This analysis was extended by generating mutant LA(1-6)-Fc decoys with Trp mutations to assess binding as a function of neutralization activity. A W50A substitution in LA1 (LA(1-6mut1)) impaired the neutralizing activity of LA(1-6)-Fc against SINV-EEEV FL93-939 but not SINV-EEEV PE-6 (FIG. 13F, FIG. 13G). However, a W89A substitution in LA2 (LA(1-6mut2)) did not lessen the neutralizing activity of LA(1-6)-Fc against either SINV-EEEV FL93-939 or PE-6. To determine which other LA domains contributed to neutralization of FL93-939, LA(1-6mut2) with additional Trp mutations were generated alone or in combination with other LA domains. As expected, LA(1-6mut2,3,5,6) did not neutralize SINV-EEEV FL93-939 infection (FIG. 13H). In contrast, LA(1-6mut2,5,6) and LA(1-6mut2,3,5) neutralized SINV-EEEV FL93-939 infection, albeit less potently than LA(1-6mut2), suggesting that LA3 and LA6 also can engage the A domain site.

To corroborate that a non-LA2 domain can recognize the A domain site, the binding of LA(1-6mut2)-Fc to EEEV FL93-939 VLPs was evaluated with or without K231 E and K232E substitutions in E2. As LA(1-6mut2) showed substantially less binding to FL93-939 E2-K231E/E2-K232E VLPs than WT FL93-939 VLPs, non-LA2 domains of VLDLR likely can bind the A domain site of EEEV FL93-939 (FIG. 13I).

### Two Sites on EEEV E2 Mediate Efficient VLDLR-Dependent Infection.

To test the functional importance of the cleft binding site, a SINV-EEEV PE-6 E2-K156A virus was generated. This mutant virus infected VLDLR-expressing K562 cells as efficiently as the parental SINV-EEEV PE-6 (FIG. 5C). A SINV-EEEV PE-6 E2-K156A/E2-K206E double mutant virus was then generated in which both the cleft and B domain binding sites are inactivated. While this mutant virus grew as efficiently as parental SINV-EEEV PE-6 in BHK-21 and Vero cells, the SINV-EEEV-PE-6 E2-K156A/E2-K206E mutant virus did not infect VLDLR-expressing K562 cells (FIG. 5C). Thus, the A domain site (E2-K231 and E2-K232) alone is insufficient to mediate VLDLR-dependent infection. These data suggest that E2-K206 at the B domain site can rescue VLDLR-dependent infection in the event of mutations within the cleft.

Next considered was whether the neighboring residues E2-H155 and E2-R157 in the cleft could compensate for the E2-K156A mutation in the context of the SINV-EEEV PE-6 strain, given that SINV-EEEV PE-6 E2-K156A used VLDLR as efficiently as the parental virus (FIG. 4H). To test whether cleft binding was incompletely abolished with the E2-K156A mutant, a SINV-EEEV PE-6 HKR→AAA virus was generated. This mutant virus showed only a moderate loss-of-infection phenotype compared to parental SINV-EEEV PE-6 in VLDLR-expressing K562 cells (FIG. 5D), suggesting that the cleft binding site is not essential for EEEV PE-6 to utilize VLDLR. Moreover, when neutralization experiments were performed with SINV-EEEV PE-6 HKR→AAA virus with Fc fusion protein decoys, inhibition was observed with VLDLR-LBD-Fc (FIG. 5E), which can still recognize two binding sites (E2 A and B domains) on this mutant virus. In contrast, LA(1-2)-Fc or LA(3-8)-Fc failed to neutralize SINV-EEEV PE-6 HKR→AAA (FIG. 5E); in the disclosed cryo-EM reconstructions, LA(1-2) or LA(3-8) bind only one other site in addition to the E1/E2 cleft (E2 A and E2 B domains, respectively). Together, these results indicate the E1/E2 cleft site is not absolutely required to efficiently bind VLDLR for EEEV strains featuring E2-K206.

Whether the cleft binding site alone was sufficient for VLDLR-dependent infection was also tested by generating SINV-EEEV PE-6 E2-K206E/E2-K231 E/E2-K232E, in which the E2 A and B domain binding sites are inactivated. While this mutant virus grew in BHK-21 cells, it poorly infected VLDLR-expressing K562 cells (FIG. 5F). These studies were extended by generating SINV-EEEV PE-6 E2-K231 E/E2-K232E (K206 and cleft binding site present); this mutant virus used VLDLR for infection with only a slightly reduced efficiency (FIG. 5F). These results are consistent with a requirement for two binding sites on EEEV (cleft+A domain, cleft+B domain, or A+B domains) for efficient VLDLR-dependent infection.

### Comparison of Receptor Engagement by Alphaviruses.

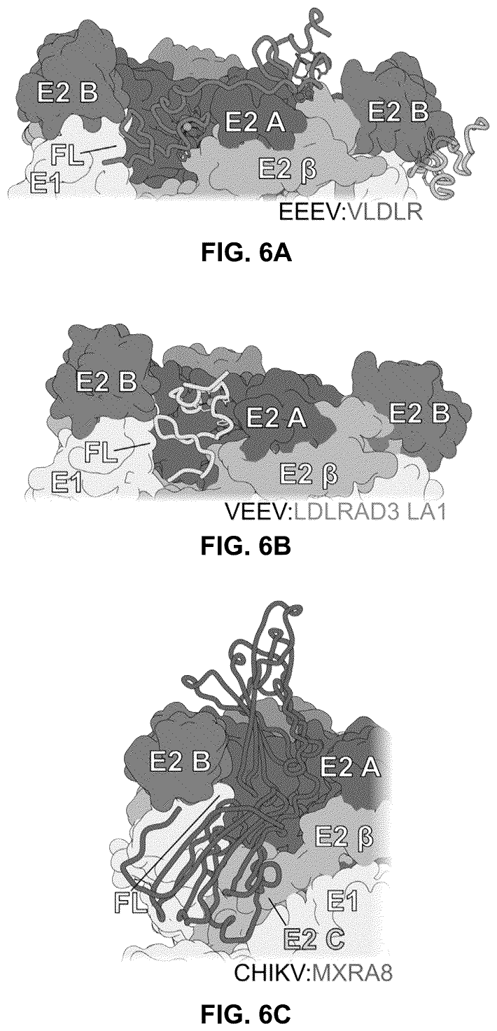

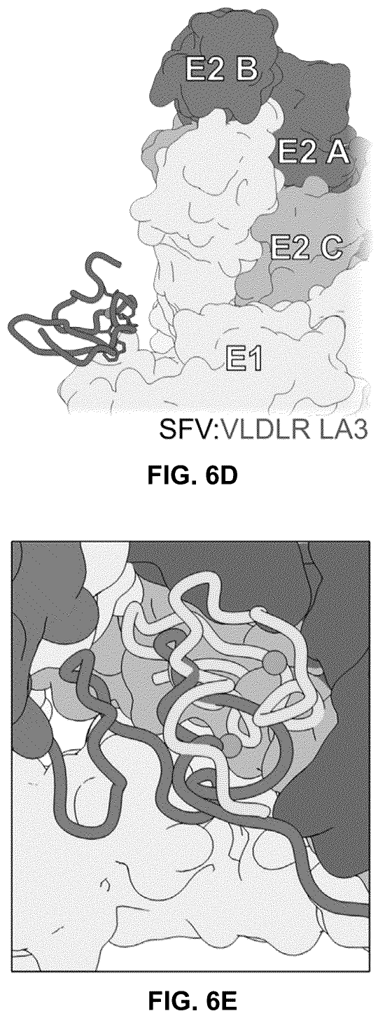

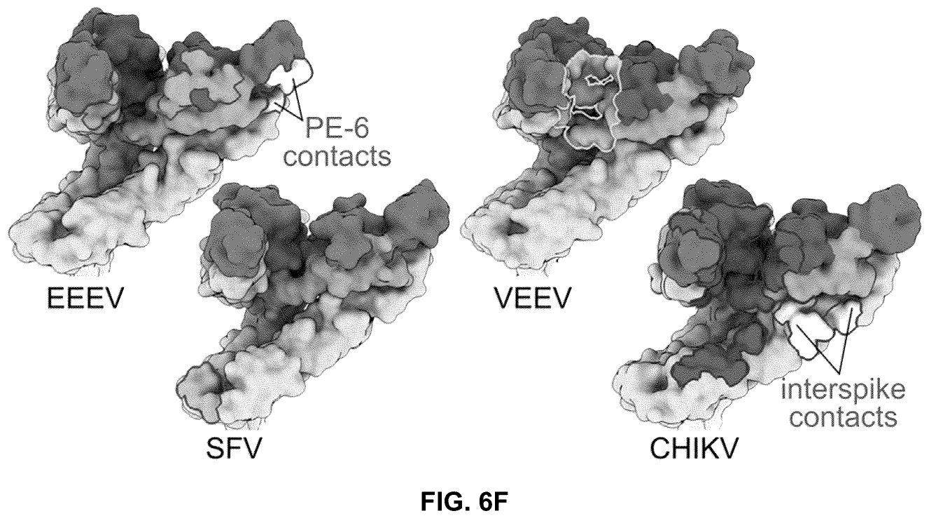

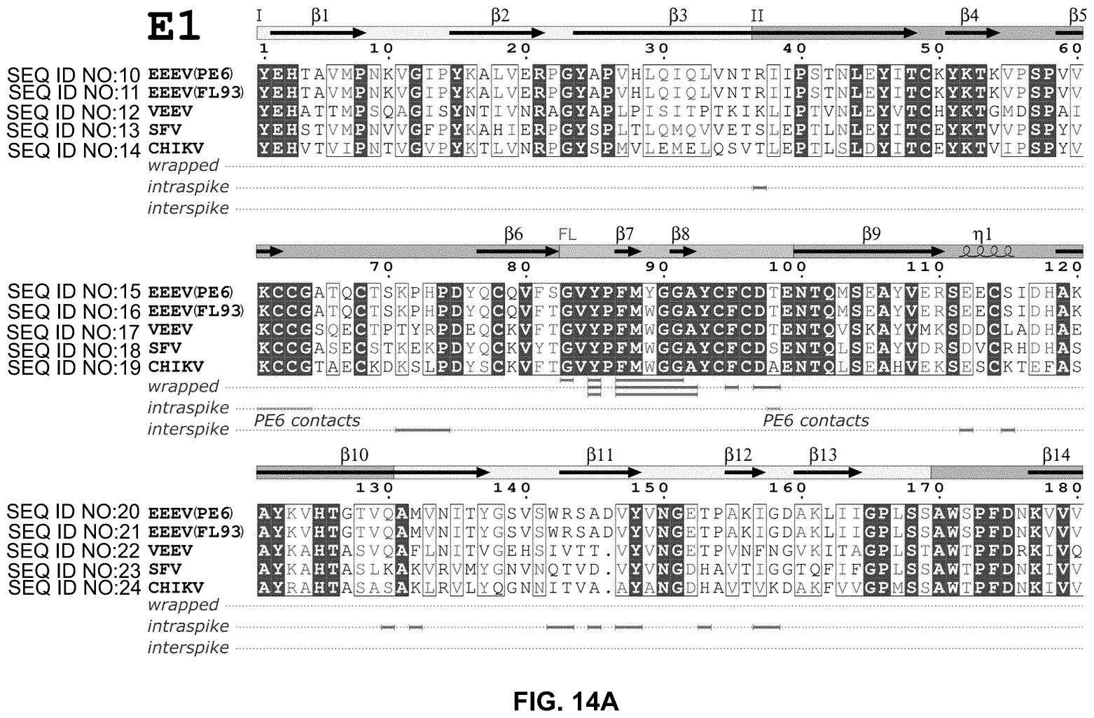

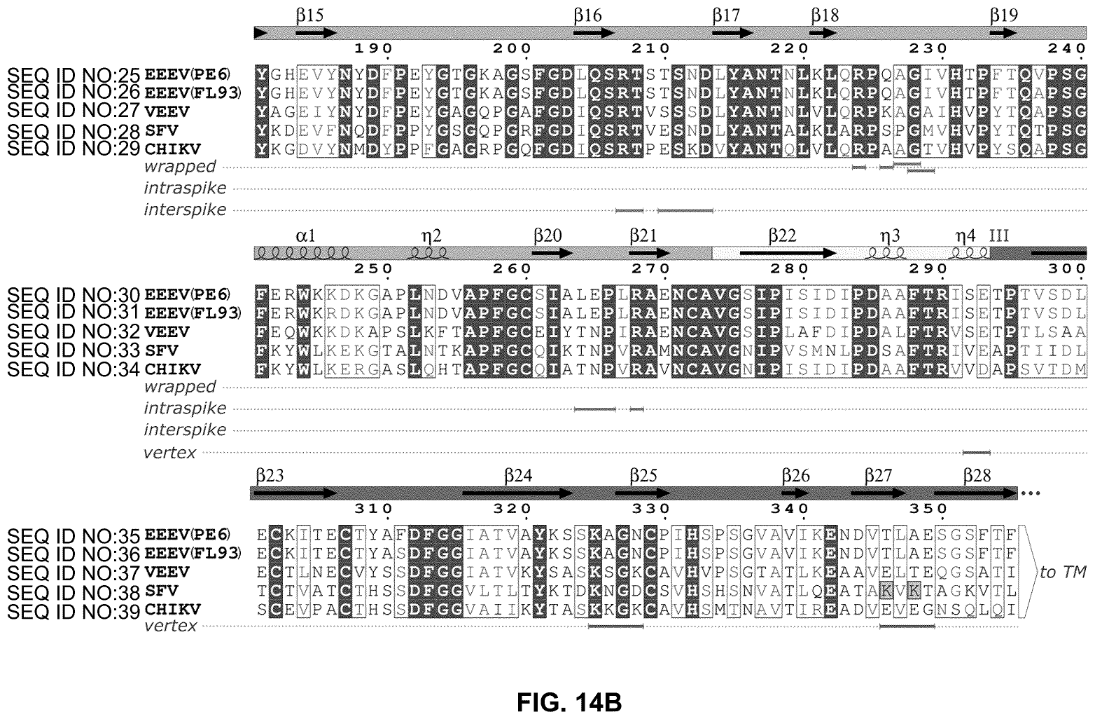

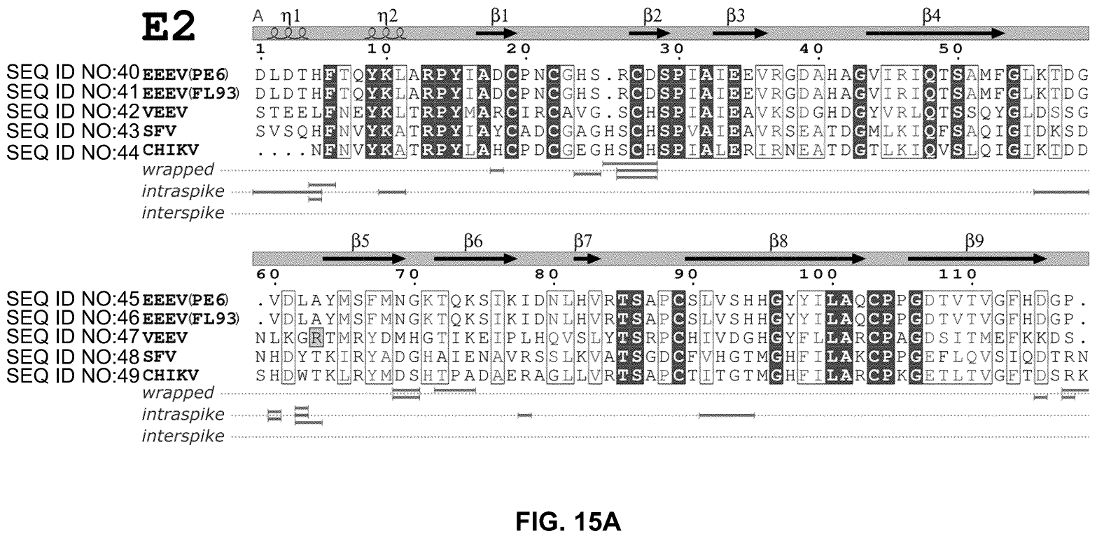

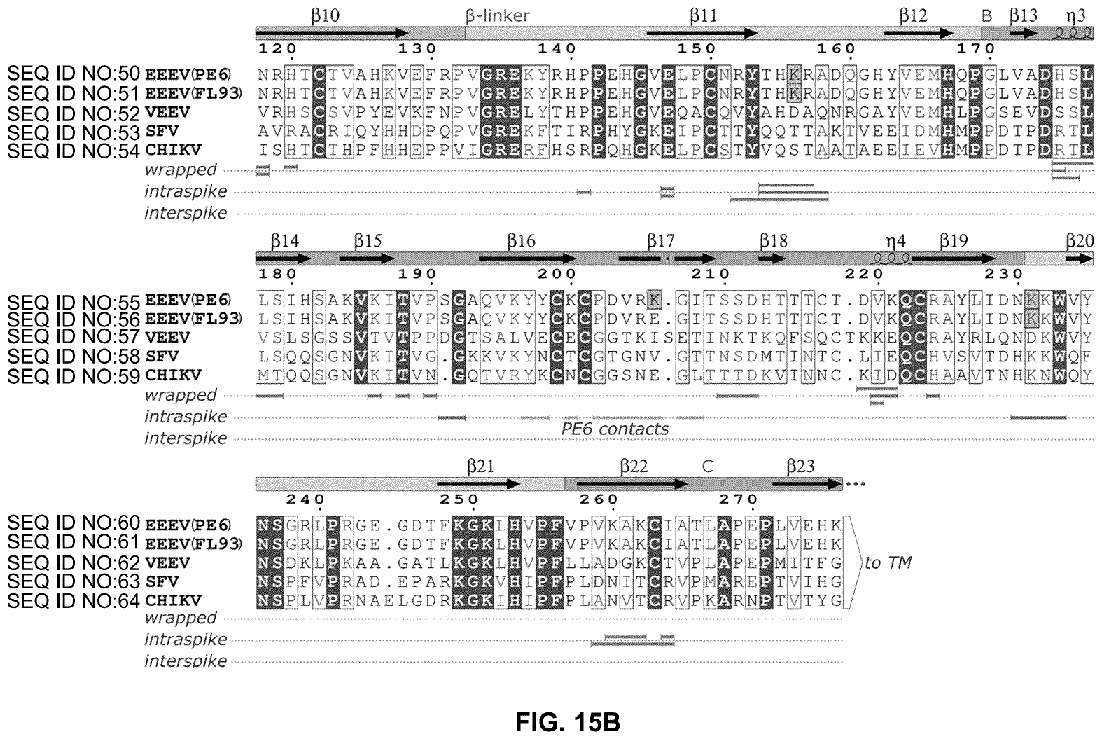

The structural interface of EEEV with VLDLR resembles yet differs from those observed for other alphavirus-receptor complexes (FIG. 14A, FIG. 14B, FIG. 15A, FIG. 15B). Whereas LA1 binds within the cleft between E1/E2 heterodimers in a manner analogous to VEEV/LDLRAD3 and CHIKV/MXRA8 (FIG. 6A, FIG. 6B, FIG. 6C), LA2 engages a unique site on a shelf atop the A domain of EEEV E2 and other LA domains engage a third site on E2 B domain in some EEEV strains (FIG. 6A). In comparison, SFV engages VLDLR LA3 outside of the cleft through domain 11 of E1 (FIG. 6D). Like EEEV, SFV can engage multiple domains of VLDLR; however, the concomitant engagement of different LA domains was not structurally or functionally resolved, and unlike EEEV, SFV uses a single site in E1 for receptor binding.

Although EEEV and VEEV can engage LA domains using their E1/E2 clefts in a roughly similar location, EEEV establishes two limited points of contact with VLDLR LA1 using the fusion loop of E1 and the HKR loop of E2. LA1 is otherwise suspended away from the back of the cleft (FIG. 6E), burying only ˜650 Å² of the viral surface (by PISA analysis) (FIG. 6F). In contrast, VEEV positions LDLRAD3 LA1 inward to form extensive hydrophobic and Van der Waals contacts at the back of the cleft (˜900 Å²) (FIG. 6E, FIG. 6F). VLDLR LA1 and LA2 together establish a larger interface (˜1,100 Å²) than LDLRAD3, and an additional ˜290 Å² is buried at the B domain site in some EEEV strains (FIG. 6F). This three-site interface still is smaller than the expansive interaction between MXRA8 and CHIKV (˜2,200 Å²), and the single VLDLR-binding site on SFV is even more limited (˜380 Å²) (FIG. 6F). Nonetheless, because EEEV and SFV can engage multiple LA domains concurrently, they bind VLDLR with higher effective affinity (K_{D} values of ˜15 nM and ˜2 nM, respectively) than CHIKV/MXRA8 (K_{D} of 84 to 270 nM) or VEEV/LDLRAD3-LA1 (K_{D} of 50 nM).

### LA(1-2)-Fc Protects Against Lethal EEEV Challenge in Mice.

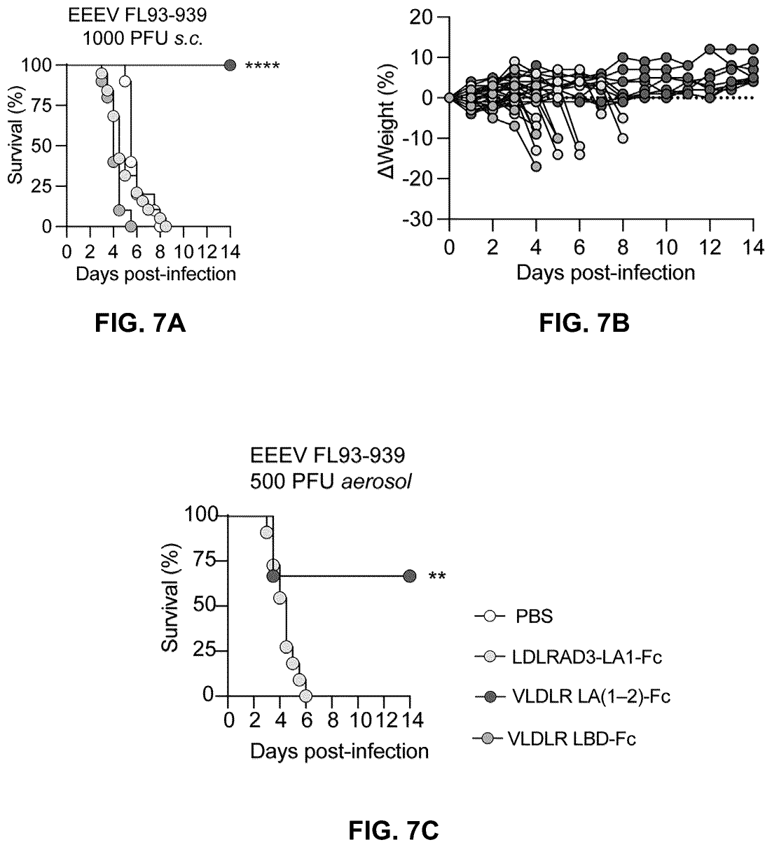

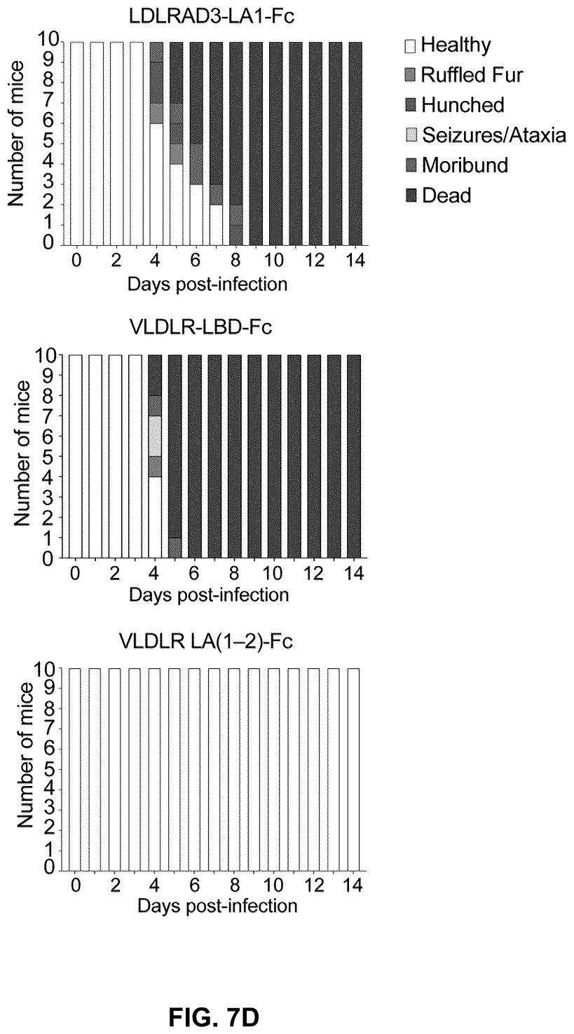

Next tested was the protective efficacy of the VLDLR-LBD-Fc decoy and LA(1-2)-Fc against EEEV in vivo. LA(1-2)-Fc was used as it retains neutralizing activity against EEEV and lacks other LA domains of VLDLR (e.g., LA5 and LA6) implicated in endogenous lipoprotein binding, which could impact bioavailability and efficacy. PBS was administered or 100 μg (˜5 mg/kg) of LDLRAD3-LA1-Fc, VLDLR LBD-Fc, or VLDLR LA(1-2)-Fc to female CD-1 mice 6 h prior to subcutaneous inoculation with 103 FFU of the authentic EEEV FL93-939 strain. All animals treated with PBS, LDLRAD3-LA1-Fc, or LBD-Fc died within 8 days of infection (FIG. 7A). Mice given VLDLR LA(1-2)-Fc were protected from lethality, weight loss, and signs of disease (FIG. 7B, FIG. 7C). Although LBD-Fc exhibited similar neutralization potency as LA(1-2)-Fc against FL93-939, its lipoprotein binding activity in vivo might have limited efficacy. Next tested was LA(1-2)-Fc in a more stringent aerosol challenge model. Mice administered VLDLR LA(1-2)-Fc were protected (70% vs 0% survival) from aerosol EEEV FL93-939 infection compared to LDLRAD3-LA1-Fc treated mice (FIG. 7D). Overall, these data highlight the protective activity of a truncated two-domain VLDLR decoy molecule against EEEV infection.

### DISCUSSION

Experiments show that EEEV uses multiple distinct sites on the E2 glycoprotein to mediate efficient VLDLR-dependent infection (FIG. 16A, FIG. 16B). Whereas 2 of the 3 identified sites in E2 are conserved in all deposited EEEV sequences present in Genbank, the PE-6 strain and some other related strains (EEEV A61-1K and Madariaga virus BeAr348998) have a rare polymorphism (E2-E206K) that enables LA domain binding to a third site on the E2-B domain. In addition, it was herein show that the five VLDLR LA domains (LA1, LA2, LA3, LA5, and LA6) that encode a conserved Trp all can participate in binding to EEEV, and observe promiscuity in LA domain interactions with each of the three EEEV binding sites: (a) EEEV engages both VLDLR LA1 and LA2, with LA1 bound within the cleft near the E1 fusion loop and LA2 positioned atop a neighboring site on the A domain of E2; (b) LA3, LA5, and LA6 all can bind to the E2 B domain site when the K206 residue is present in EEEV; (c) domain(s) other than LA1 can bind in the cleft; and (d) infection or neutralization of FL93-939 (which lacks the B domain site) with an LA(1-6) featuring an inactivating LA2 W89 mutation strongly suggests that an additional LA domain (e.g., likely LA3 or LA6) can bind the E2-A domain site. Of note, the E2-A domain site might be partially occluded by the E3 protein present on immature and partially mature virions, potentially impacting receptor engagement. No major conformational changes were observed upon LA domain binding at any of the sites in EEEV. Potential effects of the different VLDLR binding modes on downstream processes of viral entry may warrant further study.

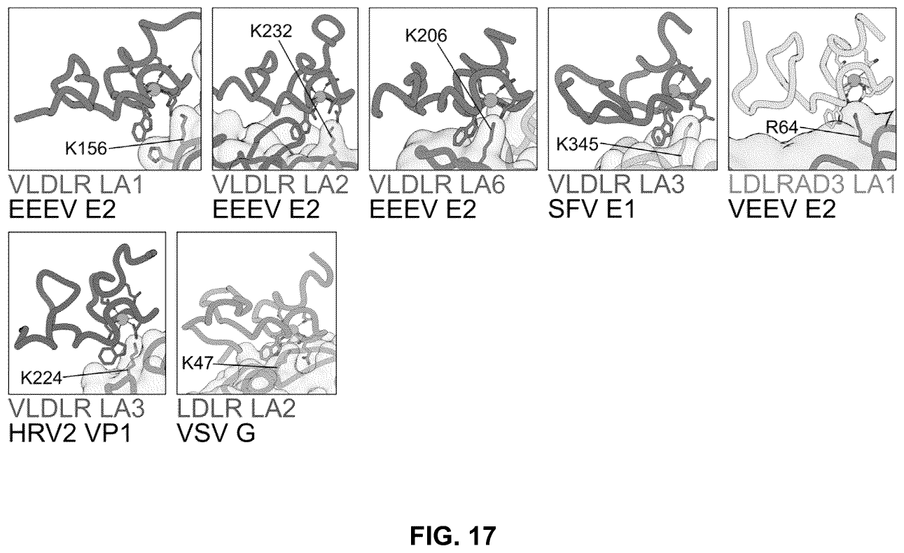

The PE-6 strain, a component of the trivalent VEEV/EEEV/WEEV VLP vaccine currently in clinical trials, features E2-K206, which allows EEEV to engage LA domains on the side of the E2 B domain. Other commonly used experimental EEEV strains (e.g., FL93-939 and FL91-469) possess E2-E206 and do not use this E2 B domain site to engage VLDLR. Thus, the third VLDLR binding site observed for EEEV PE-6 appears to be an exception. The E2-K206 residue might be a tissue culture adaptation, since the sequence of the PE-6 strain was obtained after indeterminant passage. One other EEEV isolate (A61-1K) and one sequenced Madariaga (EEE serocomplex) isolate also harbor E2-K206. Their usage of VLDLR as a receptor and the contribution of the E2 B domain may warrant further study. Regardless, experiments show how viruses can acquire multiple receptor-binding sites to co-opt repeat domains present in host receptors for high avidity interactions.

Whereas SFV binds the same VLDLR LA domains as EEEV (LA1, LA2, LA3, LA5, and/or LA6) using a single site on domain III of E1, EEEV engages VLDLR LA(1-2) more like CHIKV/MXRA8 and VEEV/LDLRAD3, as it dominantly interacts with sites on E2 and the E1 fusion loop within the E1/E2 cleft. This differential engagement of LA domains through different envelope protein sites helps to explain how distantly related alphaviruses can bind the same host receptor. LDL-receptor family members also are implicated in entry of viruses from unrelated families; in every case that has been structurally characterized, which includes vesicular stomatitis virus (VSV)/LDLR, human rhinovirus 2 (HRV2)/VLDLR, VEEV/LDLRAD3, and SFV/VLDLR, the virus has used lysine or arginine residues to target conserved aspartate, glutamate, and tryptophan residues in the calcium-coordination site of the LA repeat, suggesting convergence toward a shared receptor engagement strategy (FIG. 17). However, in the case of LA domains engaged in the E1/E2 cleft, other surfaces of the LA domain appear to augment specificity of the interaction. Notably, SFV E2 lacks the lysine (E2-K156) critical for VLDLR LA1 binding by EEEV (FIG. 14A, FIG. 14B), and EEEV E1 lacks the key lysine (E2-K345) used by SFV to target LA3 (FIG. 13C, FIG. 13D, FIG. 13E, FIG. 13F), potentially explaining their disparate modes of engagement.

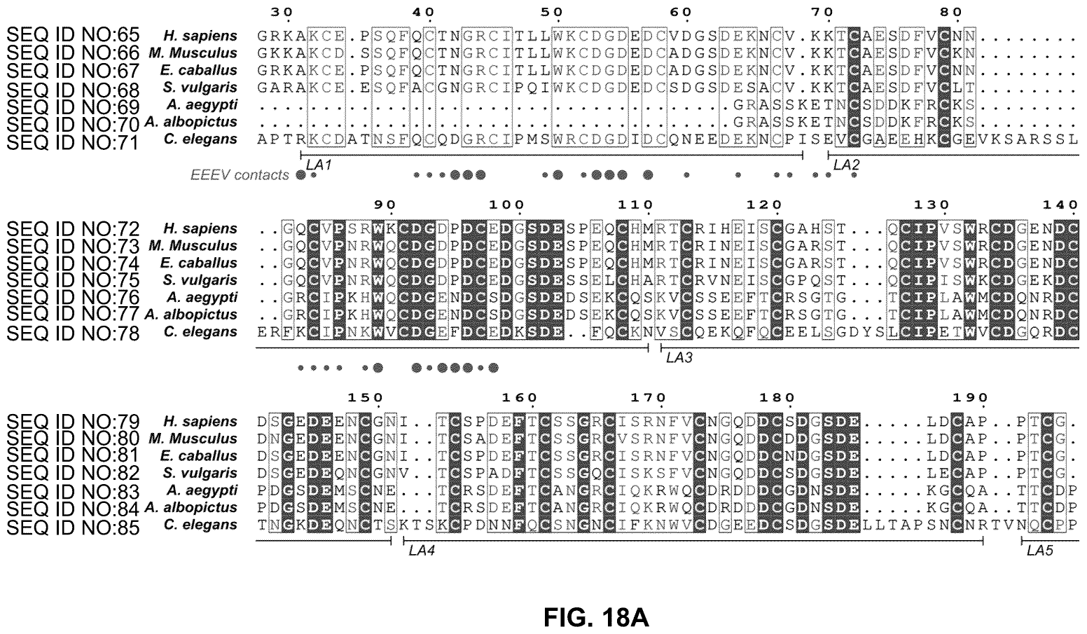

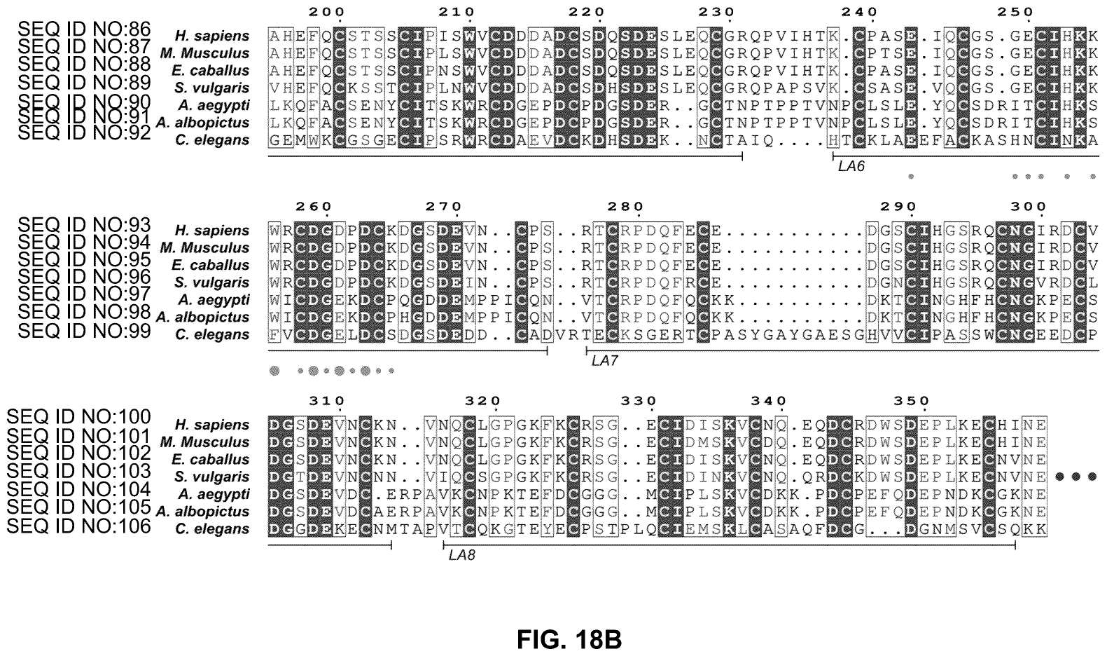

EEEV requires concurrent use of at least two LA domains of VLDLR for an avid interaction and efficient infection. While a higher affinity, single-domain receptor could still exist for EEEV, analogous to LDLRAD3-LA1/VEEV, the use of multiple LA domains could afford versatility in receptor binding. Rather than co-evolving with a single protein to achieve high-affinity binding mediated by a large binding interface, some alphavirus structural proteins may have evolved toward more flexible domain usage, engaging host receptors through a conserved structural element (e.g., the calcium-coordination site of the LA domain) with only a small interface, but at multiple sites for high avidity binding. Targeting a minimal, conserved feature also might enable interactions with related receptors in the same host or across orthologs in different species. Indeed, human LRP8 (ApoER2) and the Aedes aegypti and Aedes albopictus VLDLR orthologs can support infection of EEEV. However, structure-based sequence alignment of VLDLR orthologs from species that do (Aedes species and human) or do not (horse, avian, and C. elegans) support EEEV infection does not readily explain the differential receptor usage across evolution, as critical interacting residues within the calcium-coordination site generally are conserved across species (FIG. 18A, FIG. 18B). These structural and functional analyses informed the design of a minimal VLDLR decoy receptor that neutralizes EEEV infection and protects a subject from a lethal challenge.

### Materials and Methods

### Cells.

293T (ATCC, CRL-3216). Vero (ATCC, CCL-81), and BHK21 (ATCC, CCL-10) cells were maintained in high-glucose DMEM supplemented with 10% FBS, Gluta-MAX, 10 mM HEPES, non-essential amino acids, and penicillin-streptomycin. K562 cells (ATCC, CCL-243) were maintained in RPMI-1640 (Thermo Fisher) supplemented with 10% FBS, Gluta-MAX and 10 mM HEPES.

### Viruses.

SINV-EEEV-EGFP PE-6 (and mutants) and SINV-SFV-EGFP SFV4 were generated by replacement of structural genes of SINV-WEEV-EGFP CBA87 with EEEV PE-6 and SFV SFV4 structural genes, respectively, by PCR and Gibson assembly with EGFP expressed a structural protein fusion as described previously. The infectious cDNA clones were digested with Xhol, and the linearized vector purified with Monarch PCR & DNA Clean up Kit (New England BioLabs). One μg of linearized vector was used to generate RNA with a HiScribe SP6 RNA synthesis kit (New England BioLabs) followed by purification with Monarch RNA clean up kit. Four μgs of RNA were transfected into BHK21 cells with a GenePulser Xcell electroporator (Bio-Rad). The supernatant was harvested as the P0 stock 48 h later. The virus was passaged one additional time on BHK21 cells and titered on Vero cells. SINV-VEEV-EGFP TrD and SINV-EEEV-GFP FL93-939 has been described previously. EEEV FL93-939 was produced from a cDNA clone (a gift from S. Weaver, UTMB Galveston) as described previously.

### Mouse Studies.

All animal procedures performed at the University of Pittsburgh were carried out under approval of the Institutional Animal Care and Use Committee of the University of Pittsburgh in protocols 15066059 and 18073259. Animal care and use were performed in accordance with the recommendations in the Guide for the Care and Use of Laboratory Animals of the National Research Council. Approved euthanasia criteria were based on weight loss and morbidity. Virus inoculations were performed under anesthesia that was induced and maintained either with inhaled isoflurane or ketamine hydrochloride and xylazine, and all efforts were made to minimize animal suffering. CD-1 mice were purchased commercially (Jackson Laboratories). Subcutaneous infections with cDNA clone-derived EEEV FL93-939 were in the left rear footpad and aerosol infections were performed as previously described.

### Ectopic Expression of VLDLR Constructs.

cDNA encoding VLDLR (GenBank NP_003374.3) residues 28-873 was subcloned into a pLV-IRES-puromycin lentiviral vector with an N-terminal FLAG tag, downstream of a human β2m signal sequence. VLDLR chimeras and mutants were cloned into the pLV-VLDLR-IRES-puro or by Gibson assembly with gBlocks (IDT) and verified by Sanger sequencing or were designed and synthesized by Twist Biosciences (San Francisco, CA), into the pSFFV-IRES-puromycin backbone. Lentivirus was harvested 48 h post-transfection of HEK293T cells with donor vector, psPAX2 (Addgene #12260), and pMD2.G (Addgene #12259) in a 2:2:1 ratio using Mirus LT-1 reagent according to the manufacturer's recommended instructions. Samples were ‘spinoculated’ (800×g for 25 min) K562 cells with the lentiviral supernatants and exchanged cells into fresh media after 24 hours. Transduced cells were selected after 48 h of incubation with puromycin (2 μg/mL, Invivogen) and 7 days of culture before use in experiments. To verify FLAG-tag expression after selection, cells were incubated with anti-FLAG-Alexa Flour 647 (15009S, Cell Signaling) at a 1:200 dilution in FACS buffer (1×PBS+0.1% BSA+2 mM EDTA+0.05% NaN₃₋) for 30 min at 4° C. Following washing steps, cells were analyzed on an iQue3 flow cytometer. For some experiments, cells stained with anti-FLAG-Alexa Flour 647 were sorted for either low or high expression on a MACSQuant Tyto instrument (Miltenyi Biotec).

### Cell Infection Experiments.

Transduced K562 cells were inoculated with SINV-EGFP chimeric viruses at a multiplicity of infection (MOI) of 2 for 24 h. To assess the neutralization capacity of Fc-fusion proteins, virus was incubated with the VLDLR Fc-fusion proteins for 1 h at 37° C. before inoculation of 293T cells. Cells were harvested 16 h post-infection for flow cytometric analysis. Cells were subjected to flow cytometry in the presence of 4′,6-diamidino-2-phenylindole (DAPI, 1 μg/mL) using an iQue3 (Sartorius) and analyzed with Forecyte (Sartorius) software.

### Fc-Fusion Proteins.

The ligand binding domain (LA1-8) of human VLDLR (residues 28-355, GenBank NP_003374.3) was subcloned into a pTwist CMV β-globin expression vector downstream of a mouse IgH signal sequence encoding human IgG1 Fc separated by a linker (e.g., a short peptide <10 residues comprised of various combinations of glycine and serine) with or without an HRV 3C protease cleavage site. Sequences for constructs pT_VLDLR_LA-1-3_Fc and pTwist_VLDLR_L12-hFc are exemplified as shown in SEQ ID NO: 107 and SEQ ID NO: 108, respectively. The individual and truncated Fc-fusion constructs were generated in a similar fashion: LA1 (31-69), LA2 (70-110), LA3 (111-151), LA4(152-190), LA5(191-231), LA6(237-275), LA7(276-314), LA(1-2) (31-110), LA(2-3) (70-151), LA(3-4) (111-190), LA(4-5) (152-231), LA(5-6) (191-275), LA(1-3) (31-151), LA(1-4) (1-190), LA(1-5) (1-231), LA(1-6) (1-275). To express proteins, constructs were co-transfected with human LRPAP1 (RAP) chaperone protein (NM_002337.4, residues 1-353) at a (4:1 ratio) with Expifectamine 293 reagent into Expi293 cells (Thermo Fisher) according to the manufacturer's instructions. Supernatants were harvested four days post-transfection, centrifuged, filtered, and Fc-fusion proteins were bound to Protein A Sepharose (Thermo) in a gravity flow column. The column was washed with 25 column volumes of 1×TBS (20 mM Tris pH 8.0, 150 mM NaCl), 50 column volumes of high-salt buffer (20 mM Tris pH 8.0, 500 mM NaCl, 10 mM EDTA) to strip LRPAP, followed by 25 column volumes of 1×TBS+10 mM CaCl₂). Proteins were eluted with Pierce™ Gentle Ag/Ab Elution Buffer, pH 6.6 (Thermo Fisher) and desalted into 1×TBS (20 mM Tris pH 8.0, 150 mM NaCl) with 2 mM CaCl₂) using a PD-10 column (Cytiva). Depending on the yield, protein was concentrated with an Amicon 10 kDa centrifugal filter (Millipore). Protein purity was assessed by non-reducing SDS-PAGE followed by staining with SimpliSafe Coomasie reagent (Thermo Fisher). Gels were imaged with an iBright 1500 instrument (Thermo Fisher).

### VLPs.

EEEV PE-6 and VEEV TC-83 VLPs provided by Vaccine Research Center of the National Institutes of Allergy and Infectious Diseases.

### Domain Mapping and Quantitative BLI.

All domain mapping and quantitative affinity experiments were performed on a GatorPlus BLI and analyzed using on-board software (GatorBio) or BiaEvaluation Software (Biacore). Unless otherwise noted, all experiments were performed with 1×PBS supplemented with 1% BSA and 2 mM CaCl₂) (Running buffer). To test the avidity of receptor constructs clustered in the solid phase, 10 VLDLR-Fc fusion constructs were immobilized on anti-human IgG Fc biosensors (GatorBio #160003) for 120 sec, washed in Running buffer for 30 sec, then submerged in EEEV or SFV VLPs at a nominal concentration of ˜20 μg/mL for 360 sec to assess binding. To evaluate binding of VLDLR constructs in solution, anti-mouse IgG Fc biosensors (GatorBio #160004) were incubated ˜20 μg/mL of mEEEV-3 for 120 sec then, after washing in Running buffer for 30 sec, EEEV VLPs were captured at a nominal concentration of ˜20 μg/mL for 240 sec. For qualitative domain mapping, VLP-coated biosensors then were dipped into wells containing 1 μM of each VLDLR Fc-fusion construct; for quantitative kinetic experiments, VLP-coated biosensors were submerged into in the indicated concentrations of monovalent VLDLR fragments cleaved from the Fc using GlySERIAS (Genovis #A0-GS6) or Pierce™ HRV 3C protease (Thermo Fisher). Steady state (equilibrium) affinity was determined via on-board on-board GatorOne Software (v2.7, GatorBio).

### Mutant VLP Generation and Assessment of VLDLR-Fc Binding.

To produce mutant EEEV VLPs, a previously generated EEEV E2 alanine-scanned library was utilized, or E1 mutants of a pCAGGS vector encoding the structural proteins of EEEV (KR780-2). Select E2 mutants were transfected into Expi293 cells and supernatants were harvested after 2 days and centrifuged to remove cells and debris. VLPs were captured from the crude supernatant with mEEEV-3 coated anti-mouse IgG biosensors. Non-specific binding sites were then saturated on the anti-mouse IgG pins by incubating with 20 μg/mL of MAY-117 (an isotype control mAb). To assess and quantify binding to LA(1-2)-Fc, the EEEV VLP coated sensor tips were dipped into 1000 nM of LA(1-2)-Fc or 10 μg/mL of EEEV-3. The percent binding was quantified as the BLI signal of LA(1-2)-Fc relative to EEEV-3, with mutant VLPs normalized to that of WT VLPs.

### Mouse Infections.

Five-week old CD-1 female mice were injected intraperitoneally with a single 100 mg (˜5 mg/kg) dose of VLDLR LA(1-2)-Fc or LDLRAD3 LA1-Fc or PBS (in 200 mL PBS volume), followed 6 h later by subcutaneous inoculation with 103 plaque-forming units (PFU) of EEEV FL93-939 in the left rear footpad. Aerosol exposures were performed as previously described using the AeroMP exposure system (Biaera Technologies) inside a biological safety cabinet class III with target dose of 5×10² PFU. Mice were monitored once or twice daily for weight loss, morbidity, and mortality through 14 days post infection. Clinical signs were assigned by the following criteria: 0—healthy; 1—ruffled fur, mild behavioral changes; 2—hunched posture, significant behavioral changes; 3—seizures, ataxia, catatonia; 4—recumbent moribundity; 5—death. Mice scoring 3 or higher were immediately euthanized.

### Cryo-EM Sample Preparation.

EEEV VLPs were prepared at a nominal concentration of ˜0.7 mg/mL in PBS (pH 7.4), then incubated for 1 h on ice with 2-to-10-fold molar excess of full-length VLDLR (ACROBiosystems #VLR-H5227) or VLDLR fragments. Solutions of VLPs alone or with VLDLR were applied to glow-discharged lacey carbon grids (Ted Pella #01895-F) then flash-frozen in liquid ethane using a Vitrobot Mark IV (Thermo Fisher).

### Cryo-EM Data Collection.

Grids were loaded into a Cs-corrected FEI Titan Krios 300 kV microscope or an FEI Glacios 200 kV scope, each equipped with a Falcon 4 direct electron detector, and then imaged at a nominal magnification of 59k×(Krios) or 120k×(Glacios), resulting in a calibrated pixel size of 1.081 Å (Krios) or 1.184 Å (Glacios). Movies were collected in EER format with a total dose of ˜36-40 e⁻/Å²/movie (˜4.5 e⁻/Å²/s over 8-9 sec acquisition).

### Cryo-EM Data Processing.

EER movies were binned into 36-40 fractions (˜1.0 e⁻/Å²/f) and then pre-processed via patch motion and patch CTF correction in cryoSPARC v3.1.0. Particles were selected using a template picker then cleaned via two- and three-dimensional classification. Whole VLPs with or without VLDLR were reconstructed via homogeneous non-uniform refinement with 11 symmetry imposed. Symmetry expansion was performed and individual asymmetric units were extracted for focused three-dimensional classification without orientational sampling in Relion 3.1, and the class of highest resolution for each sample was subjected to local non-uniform refinement in cryoSPARC.

### Model Building.

Starting models for EEEV structural proteins were adapted from a previous EEEV VLP cryo-EM structure (PDB: 6XO4), and VLDLR LA domains were modeled using AlphaFold2 implemented in ColabFold. These starting components were docked into the electron density maps and then refined iteratively using Coot v0.9.5, Isolde v1.1.0, Phenix v1.19, and Rosetta scripts. Proteins, Interfaces, Structures, and Assemblies (PISA) solvent exclusion analysis was used to identify contact residues and calculate buried surface area. Structures were visualized using UCSF ChimeraX.

### Quantification and Statistical Analysis.

Statistical significance was assigned using Prism Version 8.0 (GraphPad) when P<0.05. Statistical analysis of viral infection levels was determined by one-way ANOVA with Dunnett's post-test or student's t test. Statistical analysis of in vivo experiments was determined by Kaplan-Meier survival curve analysis.

What is claimed is:

1. A composition comprising a very-low-density lipoprotein receptor (VLDLR)-based decoy receptor, wherein the VLDLR-based decoy receptor comprises:
an Fc domain; and
at least one VLDLR type A (LA) domain; and
wherein the VLDLR-based decoy receptor is an Fc-LA fusion protein.

2. The composition of claim 1, wherein the Fc domain is human IgG1.

3. The composition of claim 1, wherein the at least one VLDLR LA domain is selected from VLDLR-LA1, VLDLR-LA2, VLDLR-LA3, VLDLR-LA5, VLDLR-LA6, and combinations thereof.

4. The composition of claim 1, wherein the at least one VLDLR LA domain comprises a combination LA domain selected from LA1+LA2, LA1+LA3, LA2+LA5, LA2+LA6, LA3+LA5, and LA5+LA6.

5. The composition of claim 1, wherein the at least one VLDLR LA domain comprises a tandem LA domain selected from LA(1-2), LA(2-3), LA(3-4), LA(4-5), LA (5-6), and LA(6-7).

6. The composition of claim 1, wherein the at least one VLDLR LA domain comprises LDLRAD3 LA1.

7. The composition of claim 1, wherein one or more of the at least one VLDLR LA domains comprise a Trp (W) to Ala (A) mutation.

8. The composition of claim 1, wherein the VLDLR-based decoy receptor further comprises a VLDLRΔLBD backbone.

9. A method for treating an alphavirus infection in a subject in need thereof, the method comprising:
administering to the subject a composition comprising a very-low-density lipoprotein receptor (VLDLR)-based decoy receptor, wherein the VLDLR-based decoy receptor comprises:
an Fc domain; and
at least one VLDLR type A (LA) domain; and
wherein the VLDLR-based decoy receptor is an Fc-LA fusion protein.

10. The method of claim 9, wherein the Fc domain is human IgG1.

11. The method of claim 9, wherein the at least one VLDLR LA domain is selected from:
VLDLR-LA1, VLDLR-LA2, VLDLR-LA3, VLDLR-LA5, VLDLR-LA6, and combinations thereof;
a combination LA domain selected from LA1+LA2, LA1+LA3, LA2+LA5, LA2+LA6, LA3+LA5, and LA5+LA6;
a tandem LA domain selected from LA(1-2), LA(2-3), LA(3-4), LA(4-5), LA (5-6), and LA(6-7); and
LDLRAD3 LA1.

12. The method of claim 11, wherein one or more of the at least one VLDLR LA domains comprise a Trp (W) to Ala (A) mutation.

13. The method of claim 9, wherein the VLDLR-based decoy receptor further comprises a VLDLRΔLBD backbone.

14. The method of claim 11, wherein the alphavirus infection is selected from Eastern equine encephalitis virus (EEEV) infection, Chikungunya virus (CHIKV) infection, and Venezuelan equine encephalitis virus (VEEV) infection.

15. A method for preventing an alphavirus infection in a subject, the method comprising:
administering to the subject a composition comprising a very-low-density lipoprotein receptor (VLDLR)-based decoy receptor, wherein the VLDLR-based decoy receptor comprises:
an Fc domain; and
at least one VLDLR type A (LA) domain; and
wherein the VLDLR-based decoy receptor is an Fc-LA fusion protein.

16. The method of claim 15, wherein the Fc domain is human IgG1.

17. The method of claim 15, wherein the at least one VLDLR LA domain is selected from:
VLDLR-LA1, VLDLR-LA2, VLDLR-LA3, VLDLR-LA5, VLDLR-LA6, and combinations thereof;
a combination LA domain selected from LA1+LA2, LA1+LA3, LA2+LA5, LA2+LA6, LA3+LA5, and LA5+LA6;
a tandem LA domain selected from LA(1-2), LA(2-3), LA(3-4), LA(4-5), LA (5-6), and LA(6-7); and
LDLRAD3 LA1.

18. The method of claim 17, wherein one or more of the at least one VLDLR LA domains comprise a Trp (W) to Ala (A) mutation.

19. The method of claim 15, wherein the VLDLR-based decoy receptor further comprises a VLDLRΔLBD backbone.

20. The method of claim 15, wherein the alphavirus infection is selected from Eastern equine encephalitis virus (EEEV) infection, Chikungunya virus (CHIKV) infection, and Venezuelan equine encephalitis virus (VEEV) infection.
